(19) United States
(12) Patent Application Publication
(10) Pub. No.: US 2010/0028334 A1
(43) Pub. Date: Feb. 4, 2010

(54) COMPOSITIONS AND METHODS TO POTENTIATE COLISTIN ACTIVITY

(75) Inventors: Guillaume Cottarel, Mountain View, CA (US); Jamey Wierzbowski, Stoneham, MA (US)

Correspondence Address: RONALD I. EISENSTEIN, 100 SUMMER STREET, NIXON PEABODY LLP, BOSTON, MA, 02110, US

(73) Assignee: TRUSTEES OF BOSTON UNIVERSITY, Boston, MA (US)

(21) Appl. No.: 12/519,336

(22) PCT Filed: Dec. 13, 2007
(86) PCT No.: PCT/US07/87397

§ 371 Date: Jun. 15, 2009

Related U.S. Application Data
(60) Provisional application No. 60/875,003, filed on Dec. 15, 2006.
(60) Provisional application No. 60/963,265, filed on Aug. 3, 2007.

(51) Int. Cl. A61K 38/12; A01N 37/00; A61K 39/395; A61P 31/04; C12Q 1/68

(52) U.S. Cl. 424/130.1; 514/2; 514/9; 435/6

(57) ABSTRACT

A pharmaceutical composition comprising an antimicrobial agent and an enhancer of an antimicrobial agent, wherein the enhancer of an antimicrobial agent is an inhibitor of gene, that by inactivating the gene product potentiates the effectiveness of the antimicrobial agent. In some embodiments, the pharmaceutical composition further comprises a pharmaceutically acceptable carrier. In some embodiments, the antimicrobial agent is an antimicrobial peptide such as a polymyxin, for example but not limited to colistin. In some embodiments of the present invention provides methods to treat and/or prevent infection of a subject with a microorganism by administering a pharmaceutical composition comprising an antimicrobial agent and an enhancer of an antimicrobial agent. In some embodiments, the present invention provides methods to inhibit growth of a microorganism by administering a pharmaceutical composition comprising an antimicrobial agent and an enhancer of an antimicrobial agent.

### FIELD OF THE INVENTION

The present invention relates generally to methods and compositions to treat drug resistant bacteria, and more particularly to methods and compositions to potentate the activity of lipopeptides.

### BACKGROUND OF THE INVENTION

Increasing multidrug resistance in Gram-negative bacteria, in particular Pseudomonas aeruginosa, Acinetobacter baumannii, and Klebsiella pneumoniae, presents a critical problem. Limited therapeutic options have forced infectious disease clinicians and microbiologists to reappraise the clinical application of colistin, a polymyxin antibiotic discovered more than 50 years ago. Colistin is now one of the last drug resorts to fight Gram-negative bacteria.

The world is facing an enormous and growing threat from the emergence of microorganisms that are resistant to a wide range of currently available antibiotics. For example, infections caused by utility drug resistant gram-negative bacteria, particularly Pseudomonas aeruginosa and Acinetobacter baumannii, are becoming a critical challenge in compromised hospital patients (e.g. patients in intensive care unite, patients with cystic fibrosis or diffuse panbroncholitis), and with seemingly trivial infections in sites such as middle and central ear and eyes. The level of resistance to front line anti-pseudomonal agents is alarmingly high. Of particular concern are reports on antibiograms of P. aeruginosa and A. baumannii in hospital outbreaks, where colistin, a member of the polymyxin class of antibiotics, is the only effective antibiotic. Unfortunately, polymyxin-resistant P. aeruginosa has been isolated from patients with eye infections, ear infections, and particularly in the sputum of patients with cystic fibrosis. The appearance of polymyxin-resistant gram-negative pathogens is of great concern.

Colistin is a polymyxin antibiotic produced by certain strains of Bacillus polymyxa. Colistin is effective against Gram negative bacilli, except Proteus and Burkholderia cepacia and is particularly effective against multi-drug resistant isolates of Pseudomonas aeruginosa, Acinetobacter baumannii and Klebsiella pneumoniae. Colistin interacts with the bacterial cytoplasmic membrane, changing its permeability therefore causing leakage of the cell contents.

However, despite the ability of colistin to kill multi-drug resistant bacteria, its use in treating patients is highly limited by the fact that it is highly neurotoxic and nephrotoxic and results in adverse renal effects. Colistin must be administered parenterally as it is not absorbed by the gastrointestinal tract, mucous membranes or intacted or denuded skin. Colistin administration, particularly intravenously, was abandoned due to a high incidence of nephrotoxicity and neurotoxicity. In regard of the emergence of bacteria resistant to major classes of antibiotics and of the lack of new classes of antibiotics, colistin is re-appearing as a valuable antibacterial therapeutic agent. In fact it is being reintroduced in clinical practice due to the emergence of multidrug-resistant gram-negative bacteria therefore reflecting the critical situation to cure some bacterial infection.

Colistin, also called Colimycin or polymycin E, a cyclic lipopeptide, penetrates the cell wall of gram negative bacteria by a self induced mechanism by chelating divalent ions, it destabilizes the wall and can insinuate into it. It perforates the cell wall, causing distortion of this structure and the release of intracellular constituents in the outside. Colistin appears to re-emerge as recent clinical findings have been published, focusing on evaluation of efficacy, emerging resistance and potential toxicities. The use of colistin at current therapeutic effective doses is often associated with neurotoxicity and nephrotoxicity. Therefore it would be desirable to reduce the toxicity of colistin.

Therefore, there is great need in the art to for an effective therapy against multi-drug resistant microorganisms, in particular gram-negative bacteria and multi-drug resistant gram-negative bacteria, for example, polymyxin-resistant gram-negative bacteria such as P. aeruginosa and A. baumannii, or to increase the sensitivity of microorganisms to treatment.

### SUMMARY OF THE INVENTION

The present invention relates to methods and compositions to potentate the efficacy of antimicrobial agents, for example antimicrobial peptides. The inventors have discovered that by inactivating certain genes, the effectiveness of antimicrobial agents, such as antimicrobial peptides is increased. Accordingly, the present invention relates to inhibitors of such genes as enhancers of antimicrobial agents. In alternative embodiments, the present invention relates to a composition comprising one or more antimicrobial agents with one or more enhancers of antimicrobial agents.

As disclosed herein, the inventors have discovered a method of reducing the toxicity of antimicrobial agents, such as for example, Colistin by co-administering an enhancer of the antimicrobial agent, for example an inhibitor of the gene products as disclosed herein in Table 4 or Table 2.

Using a functional genomics approach, the inventors discovered that inactivation of specific gene products render the bacteria more susceptible to colistin, a potent but toxic antibiotic. For example, the inventors demonstrate that deletion of the ubiH and iscS loci render the bacterial cells more sensitive to colistin. The inventors also demonstrate that that potassium tellurite, an inhibitor of IscS, potentiates colistin efficacy. Accordingly, the inventors have discovered different combination therapies with colistin, and their use with a wide variety of major classes of antibiotics.

Another aspect of the present invention relates to methods to screen for gene products, which when inactivated, potentiate the effect of antimicrobial agents, such as antimicrobial peptides. Another aspect of the invention relates to identification of inhibitors of such genes, and thus identification of enhancers of antimicrobial agents, such as antimicrobial peptides. Another aspect of the present invention relates to a pharmaceutical formulation comprising a composition of at least one antimicrobial agent, such as an antimicrobial peptide, and at least one enhancer to the antimicrobial agent. In such an embodiment, the enhancer of the antimicrobial agent is an inhibitor of a gene product, which by inactivating the gene product potentiates the effectiveness of the antimicrobial agent. For example, the enhancer of the antimicrobial agent, such as an inhibitor of one of the genes listed in Table 1 or 4 as disclosed herein potentiates the efficacy of the antimicrobial agent, e.g. peptides such as colistin, to a greater extent than if either was used alone (synergy), and vice versa, the antimicrobial agent potentiates the effect of the enhancer of antimicrobial peptide, referred to as bi-directional synergy. In some embodiments, the enhancer of antimicrobial agent may not have any antimicrobial effect when used on its own, whereas when such enhancer of an antimicrobial agent is used concurrently with an antimicrobial agent, it may have antimicrobial activity.

In a further embodiment, the present invention relates to the use of a composition comprising an antimicrobial agent, such as an antimicrobial peptide, and an enhancer to the antimicrobial agent for the treatment of a subject. In one such embodiment, the composition comprising an antimicrobial agent and an enhancer thereof is used to prevent and/or inhibit the growth of a microorganism. In alternative embodiments, the pharmaceutical formulations of the present invention are used for the treatment and/or prophylaxis of an infection in a subject.

In some embodiments, the antimicrobial agent is a peptide, such as a lipopeptide, and in another embodiment, the lipopeptide is a cyclic lipopeptide. In some embodiments, the lipopeptide or cyclic lipopeptide is a polymyxin or belongs to the polymyxin class of antibiotics or derivatives or analogues thereof, which are defined in more detail below. In some embodiments, the polymyxin is polymyxin A, B1, B2, D1, D2, E1, E2, F, G, M, P, S and/or T or any derivative, analogue or variant thereof. In some embodiments the polymyxin is a colistin, for example, but not limited to colistin A and/or colistin B. In some embodiments, the polymyxin is in the form of a salt, for example a methoane sulfonate or sulfate salt or a colistin salt.

Other antimicrobial agents can be used, for example but not limited to, small molecules, peptides, peptidomimetics, chemicals, compounds, and any entity that inhibits the grown and/or kills a microorganism.

In some embodiments, the gene products, which when inactivated potentiate antimicrobial agents effectiveness are, for example but not limited to the genes as shown in Table 1, for example, agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or homologues, variants and fragments thereof. In some embodiments, the gene products are atpA, atpH, betB, guaA, guaB, lipA, lysA, rpiA, and/or trxA or homologues, variants and fragments thereof.

In some embodiments, the enhancers of antimicrobial agents, such as antimicrobial peptides are for example, compounds that inhibit the gene products which when inactivated potentiate the antimicrobial agent, for example antimicrobial peptides. Examples of such inhibitors are for example but not limited to, mefloquine, venturicidin A, antimycin, myxothiazol, stigmatellin, diuron, iodoacetamide, potassium tellurite hydrate, aDL-vinylglycine, N-ethylmaleimide, L-allyglycine, diaryquinoline, betaine aldehyde chloride, acivein, psicofuraine, buthionine sulfoximine, diaminopemelic acid, 4-phospho-D-erythronhydroxamic acid, motexafin gadolinium and/or xycitrin or modified versions or analogues thereof. In particular embodiments, the enhancer of antimicrobial agents is mefloquine or antimycin, myxothiazol, stigmatellin, diuron, iodoacetamide, potassium tellurite hydrate, aDL-vinylglycine, N-ethylmaleimide, L-allyglycine. The present invention also provides methods for the modification of the enhancers of antimicrobial peptides described herein, using structure-based design methods. Also encompassed in the present invention is the use of structure-based design methods to design a single molecule that comprises both an antimicrobial agent, for example an antimicrobial peptide and an enhancer of antimicrobial agents described herein.

In further embodiments, the enhancers of antimicrobial agents are any molecule, compound and/or drug which inhibits and/or inactivates a gene that results in the potentiation of an antimicrobial agent, such as, for example an antimicrobial peptide. In some embodiments, such inhibitors to the gene products include antibodies (polyclonal or monoclonal), neutralizing antibodies, antibody fragments, chimeric antibodies, humanized antibodies, recombinant antibodies, peptides, proteins, peptide-mimetics, aptamers, oligonucleotides, hormones, small molecules, nucleic acids, nucleic acid analogues, carbohydrates or variants thereof that function to inactivate the nucleic acid and/or protein of the gene products identified herein, and those as yet unidentified. Nucleic acids include, for example but not limited to, DNA, RNA, oligonucleotides, peptide nucleic acid (PNA), pseudo-complementary-PNA (pcPNA), locked nucleic acid (LNA), RNAi, microRNAi, siRNA, shRNA etc. The inhibitors can be selected from a group of a chemical, small molecule, chemical entity, nucleic acid sequences, nucleic acid analogues or protein or polypeptide or analogue or fragment thereof.

In some embodiments, the enhancer of the antimicrobial agent as disclosed herein does not have any effect on decreasing cell viability when it is used by itself. In some embodiments, an enhancer of the antimicrobial agent is selected based on its ability to enhance the effect or activity of the antimicrobial agent. In some embodiments therefore, an enhancer of the antimicrobial agent may not have any anti-pathogenic effects or ability to decrease cell viability when used itself, and thus will have no antibiotic or no antimicrobial activity when used on its own, but when such enhancer of the antimicrobial agent is used concurrently with an antimicrobial agent, such as, for example with colistin, the enhancer of the antimicrobial agent functions to enhance the activity of the antimicrobial agent.

In some embodiments, the microorganisms of the invention are bacterium. In some embodiments, the bacteria are gram positive and gram negative bacteria. In some embodiments, the bacteria are multi-drug resistant bacterium. In further embodiments, the bacteria are polymyxin-resistant bacterium. Examples of gram-negative bacteria are for example, but not limited to P. aeruginosa, A. baumannii, Salmonella spp, Klebsiella pneumonia, Shigella spp. and/or Stenotrophomonas maltophilia.

In some embodiments, the pharmaceutical compositions of the invention are administered in coformulations with one or more other antibiotics or therapeutic agents, for example but not limited to aminoglycosides, carbapenemes, cephalosporins, cephems, glycoproteins fluroquinolones/quinolones, oxazolidinones, penicillins, streptogramins, sulfonamides and/or tetracyclines.

In a further embodiment, the invention provides methods of administration of the compositions and/or pharmaceutical formulations of the invention and include any means commonly known by persons skilled in the art. In some embodiments, the subject is any organism, including for example a mammalian, avian or plant. In some embodiments, the mammalian is a human, a domesticated animal and/or a commercial animal.

One aspect of the present invention relates to a composition comprising an antimicrobial agent and an enhancer to the antimicrobial agent, wherein the enhancer to the antimicrobial agent is an inhibitor of a gene product that by inactivating the gene product potentiates the effectiveness of the antimicrobial agent. In some embodiments, the antimicrobial agent is a peptide, for example but not limited to a lipopeptide such as a cyclic lipopeptide. In some embodiments, a cyclic lipopeptide is a polymyxin class of antibiotic or derivative thereof, for example but not limited to a polymyxin is selected from the group of polymyxin A, B1, B2, D1, D2, E1 and/or E2, F, G, M, P, S and/or T. In some embodiments, the polymyxin is selected from polymyzin B1 and/or polymyxin B2 or from colistin A and/or colistin B.

In some embodiments, the composition as disclosed herein comprises colistin in the form of a colistin salt, for example methane sulphonate and/or sulfate salt.

In some embodiments, the compositions comprises an enhancer of an antimicrobial agent which is an agent which inactivates a gene or gene product, for example a gene or gene product is selected from a group consisting of agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or homologues, variants or fragments thereof. In some embodiments the compositions comprises an enhancer of an antimicrobial agent, which is an agent that inactivates a gene or gene product which is encoded by any of the genes listed in Table 1 or Table 4. In some embodiments, an enhancer of an antimicrobial agent is an inhibitor of a gene listed in Table 1 or 4, for example suitable inhibitors useful as enhancers of antimicrobial agents as disclosed herein include, but are not limited to mefloquine, venturicidin A, diaryquinoline, betaine aldehyde chloride, acivein, psicofuraine, buthionine sulfoximine, diaminopemelic acid, 4-phospho-D-erythronhydroxamic acid, motexafin gadolinium and/or xycitrin or modified versions or analogues thereof.

In some embodiments, the composition as disclosed herein comprises an enhancer of an antimicrobial agent which inhibits the gene product is atpA, atpF or atpH or homologues, variants or fragments thereof, and the enhancer of the antimicrobial agent or inhibitor is mefloquine and/or venturicidin A and/or diaryquinoline or modified versions or analogues thereof.

In some embodiments, the composition as disclosed herein comprises an enhancer of an antimicrobial agent which inhibits the gene product betB or homologues or variants thereof, and the inhibitor is betaine aldehyde chloride or modified versions or analogues thereof.

In some embodiments, the composition as disclosed herein comprises an enhancer of an antimicrobial agent which inhibits the gene product is guaA or guaB or homologues or variants thereof, and the inhibitor or enhancer of the antimicrobial agent is acivin and/or psicofluranine or modified versions or analogues thereof.

In some embodiments, the composition as disclosed herein comprises an enhancer of an antimicrobial agent which inhibits the gene product is LipA or homologues or variants thereof, and the inhibitor or enhancer of antimicrobial agent is buthionine sulfoximine or modified versions or analogues thereof.

In some embodiments, the composition as disclosed herein comprises an enhancer of an antimicrobial agent which inhibits the gene product is LysA or homologues or variants thereof, and the inhibitor or enhancer of antimicrobial agent is diaminopimelic acid or modified versions or analogues thereof.

In some embodiments, the composition as disclosed herein comprises an enhancer of an antimicrobial agent which inhibits the gene product is rpiA or homologues or variants thereof, and the enhancer is inhibitor or enhancer of antimicrobial agent is 4-phospho-D-erythronhydrixamic acid or modified versions or analogues thereof.

In some embodiments, the composition as disclosed herein comprises an enhancer of an antimicrobial agent which inhibits the gene product is trxA or homologues or variants thereof, and the inhibitor or enhancer of antimicrobial agent is motexafin gadolinium and/or xycitrin acid or modified versions or analogues thereof.

In some embodiments, the enhancer of antimicrobial agent inhibitor comprises a small molecule, nucleic acid, nucleic acid analogue, peptide, ribosome, antibody, and variants and fragments thereof. In some embodiments, the nucleic acid is DNA, RNA, DNA/RNA hybrids, a triple helix nucleic acid, an antisense nucleic acid, ribozyme or an RNAi or a homologue or fragment thereof. In some embodiments, the nucleic acid analogues comprises peptide nucleic acid (PNA), pseudo-complementary PNA (pcPNA), locked nucleic acid (LNA) and variants thereof.

In some embodiments, the compositions as disclosed herein are useful in the inhibition of growth and/or decrease in viability of a microorganism, for example a bacterium. In some embodiments, the bacterium is a gram-positive bacterium, and in alternative embodiments, the bacterium is a gram-negative bacterium. In some embodiments, the microorganism is a multi-drug resistant microorganism, for example but not limited to a multi-drug resistant microorganism is resistant to at least one member of the polymyxin class of antibiotics or derivatives or analogues thereof. In some embodiments, a multi-drug resistant microorganism is polymyxin resistant Pseudomonas aeruginosa, Acinetobacter baumannii, Stenotrophomonas maltophilia, Salmonella spp. and/or Klebsiella pneumonia and/or Shigella spp. In some embodiments, the multi-drug resistant microorganism is resistant to colistin and/or a colistin salt.

In some embodiments, the composition as disclosed herein, can be administered at different times, for example a antimicrobial agent can be administered prior to, or simultaneously with, the administration of the enhancer of the antimicrobial agent or an inhibitor of the gene product.

Another aspect of the present invention relates to a pharmaceutical formulation comprising the composition as disclosed herein and a pharmaceutically acceptable carrier. In some embodiments, the composition as disclosed herein us useful to treat an infection caused by a microorganism. In some embodiments, the use of the composition as disclosed herein comprises the amount of the antimicrobial agent is at least 25% less than the same antimicrobial agent in an isogenic cell except for the addition of the enhancer of antimicrobial agent without a reduction of antimicrobial effect.

Another aspect of the present invention relates to a method of killing and/or preventing or inhibiting growth of a microorganism comprising administering to a subject in need thereof an effective amount of a pharmaceutical formulation of the composition as disclosed herein. In some embodiments, the present invention provides a method of treatment and/or prophylaxis of an infection caused by a microorganism comprising steps of administering to a subject in need thereof an effective amount of a pharmaceutical formulation of the composition as disclosed herein.

In some embodiments, the compositions and methods as disclosed herein are administered to a subject, for example, where a subject is mammalian, avian or a plant. In some embodiments the subject is a mammalian, for example a human. In further embodiments, the mammalian is a domesticated animal.

In some embodiments, the compositions and methods as disclosed herein are administered to a subject to decrease the viability of a microorganism, for example a bacterium, such as a gram-positive bacterium or a gram-negative bacterium.

In some embodiments, the compositions and methods as disclosed herein are administered to a subject to treat an infection, for example infections such as, but not limited to, infection is selected from the group consisting of bacterial wound infections, mucosal infections, enteric infections, septic conditions, infectious in airways, cerebrospinal fluid, blood, eyes and skin. In some embodiments, an infection is caused by gram-negative bacteria. In some embodiments an infection is caused by multi-drug resistant gram negative bacteria, such as for example multi-drug resistant microorganism is resistant to at least one member of the polymyxin class of antibiotics and synthetic derivatives thereof. In some embodiments, the compositions and methods as disclosed herein are administered to a subject that is infected with a microorganism, such as, for example, a microorganisms are selected from a group comprising; Pseudomonas aeruginosa, Acinetobacter baumannii, Stenotrophomonas maltophilia, Salmonella spp and/or Klebsiella pneumonia and/or Shigella spp.

Another aspect of the present invention relates to the use of an antimicrobial agent and an enhancer to the antimicrobial agent, wherein the enhancer inhibits a gene product of the treatment or prophylaxis of an infection caused by a microorganism, wherein the antimicrobial agent and the inhibitor interact synergistically against the microorganism. In some embodiments, the infection is selected from the group consisting of bacterial wound infections, mucosal infections, enteric infections, septic conditions, infectious in airways, cerebrospinal fluid, blood, eyes and skin.

Another aspect of the present invention relates to the use of an antimicrobial agent and an enhancer of an antimicrobial agent, or an inhibitor to a gene product in the manufacture of a medicament for inhibiting or preventing production of at least one pathogenic factor by a microorganism. In some embodiments, the use of an antimicrobial agent and an inhibitor to a gene product in the manufacture of a medicament for inhibiting or preventing production of at least one pathogenic factor by a microorganism in a subject being treated with a medicament comprising a antimicrobial agent and the inhibitor.

Another aspect of the present invention relates to a pharmaceutical formulation comprising the composition as disclosed herein, wherein the enhancer of the antimicrobial agent enhances the anti-pathogenic activity of the antimicrobial agent.

Another aspect of the present invention relates to a method for identifying gene products, wherein inactivation of the gene products potentiates antimicrobial agent activity, the method comprising the steps of; (a) mutating one or more genes in a cell, (b) contacting the cell with the antimicrobial agent, incubating the cell for a sufficient amount of time to allow for growth, and (c) assessing the number of cells, wherein the number of cells is compared to steps (a)-(c) performed on a non-mutated cell, wherein the decrease in numbers of cells identifies a gene product that when inactivated potentiates antimicrobial peptide activity. In some embodiments, the cell used in the method to identify antimicrobial agents is a bacterium, for example E. Coli. In alternative embodiments, the cell is selected from a group consisting of: Bacillus cereus, Bacillus anthracis, Bacillus cereus, Bacillus anthracis, Clostridium botulinum, Clostridium difficle, Clostridium tetani, Clostridium perfringens, Corynebacteria diptheriae, Enterococcus (Streptococcus D), Listeria monocytogenes, Pneumococcal infections (Streptococcus pneumoniae), Staphylococcal infections and Streptococcal infections; Gram-negative bacteria including Bacteroides, Bordetella pertussis, Brucella, Campylobacter infections, enterohaemorrhagic Escherichia coli (EHEC/E. coli 0157:17), enteroinvasive Escherichia coli (EIEC), enterotoxigenic Escherichia coli (ETEC), Haemophilus influenzae, Helicobacter pylori, Klebsiella pneumoniae, Legionella spp., Moraxella catarrhalis, Neisseria gonorrhoeae, Neisseria meningitidis, Proteus spp., Pseudomonas aeruginosa, Salmonella spp., Shigella spp., Vibrio cholera and Yersinia; acid fast bacteria including Mycobacterium tuberculosis, Mycobacterium avium-intracellulars, Mycobacterium johnei, Mycobacterium leprae, atypical bacteria, Chlamydia, Myoplasma, Rickettsia, Spirochetes, Treponema pallidum, Borrelia recurrentis, Borrelia burgdorfii and Leptospira icterohemorrhagiae, Actinomyces, Nocardia, P. aeruginosa, A. baumannii, Salmonella spp., Klebsiella pneumonia, Shigella spp. and/or Stenotrophomonas maltophilia and other miscellaneous bacteria. In some embodiments, the antimicrobial agent used in the methods to identify a gene product that when inactivated potentiates antimicrobial peptide activity is any antimicrobial agent commonly known by persons of ordinary skill in the art or are disclosed herein. In some embodiments, in the methods to identify gene product that when inactivated potentiates antimicrobial peptide activity, mutation results in inactivation of the gene or gene product.

### BRIEF DESCRIPTION OF FIGURES

FIG. 1 shows a schematic drawing of the screen shown in a 96 well plate format for simplicity, the screen was done in a 384 well plate format. The arrow points to the expected result. Example of digital image analysis with the iscS and ubiH mutants as example.

FIG. 2 shows a flow chart of the genetic and chemical screen leading to a combination therapy between colistin and potassium tellurite. Mutants are listed in table 1 in the supplement section.

FIG. 3 shows a heat map of gene homology of the identified gene loci in different bacteria. The conserved genes loci are dark, and less conserved gene loci are light in color.

FIG. 4 shows an example of colistin and mefloquine synergy in a minimum inhibitory concentration (MIC) assay in E. coli. As the concentration of mefloquine increases, the minimum inhibitory concentration of colistin decreases.

FIG. 5 shows a phenotypic response to colistin. FIG. 5A shows the log change in colony forming units per ml (CFU/mil) of wild-type, BW25113 and ubiH and iscS mutant E. coli cells (mean±s.d.) in presence or absence of colistin. The cells were incubated with colistin at a final concentration of 1.25 ug/ml. At regular time points (3 and 6 hours), aliquots were taken and serially diluted into PBS. 10 ul of cells were plated onto LB agar [no colistin] plates. Number of colonies were counted after overnight incubation at 37 C. Colony count was normalized to get the same starting amount of cells for all strains. FIG. 5B shows the phenotypic response to colistin and potassium tellurite. Log change in colony forming units per ml (CFU/ml) of wild-type, BW25113 E. coli cells (mean±s.d.) in presence or absence of colistin and potassium tellurite. Cell were grown in the absence or presence of colistin (1.25 ug/ml), potassium tellurite (1.6 ug/ml) alone and combined. At regular time points (3 and 6 hours)) aliquots were taken and serially diluted into PBS. 10 ul of cells were plated onto LB agar [no colistin] plates. Number of colonies were counted after overnight incubation at 37 C. Colony count was normalized to get the same starting amount of cells for all strains.

### DETAILED DESCRIPTION OF THE INVENTION

The present invention relates to methods and compositions to potentate the efficacy of antimicrobial agents such as antimicrobial peptides. The inventors have discovered that by inactivating certain genes, the effectiveness of an antimicrobial agent, such as an antimicrobial peptide is increased. Accordingly, the present invention relates to inhibitors of such genes as enhancers of antimicrobial agents, for example antimicrobial peptides. In alternative embodiments, the present invention relates to compositions comprising one or more antimicrobial agents, such as antimicrobial peptides, with one or more enhancers thereof.

The present invention relates to methods and compositions comprising antimicrobial agents, such as peptides and enhancers thereof, such as enhancers of antimicrobial peptides. In particular embodiments, the antimicrobial agents are antimicrobial peptides, and in some embodiments, the antimicrobial peptides are lipopeptides, in particular cyclic lipopeptides. Enhancers to antimicrobial agents, for example enhancers of antimicrobial peptides can include nucleic acids, peptide, nucleic acid analogues, phage, phagemids, polypeptides, peptidomimetics, antibodies, small or large organic or inorganic molecules or any combination of the above. The enhancers to the antimicrobial agents can be naturally occurring or non-naturally occurring (e.g., recombinant) and are sometimes isolated and/or purified.

In some embodiments, the enhancers of antimicrobial agents such as antimicrobial peptides are for example, compounds that inhibit the gene products which when inactivated potentiate the antimicrobial agents. Examples of such inhibitors are for example but not limited to, mefloquine, venturicidin A, diaryquinoline, betaine aldehyde chloride, acivein, psicofuraine, buthionine sulfoximine, diaminopemelic acid, 4-phospho-D-erythronhydroxamic acid, motexafin gadolinium and/or xycitrin or modified versions or analogues thereof. In particular embodiments, an enhancer of antimicrobial agents is mefloquine

In some embodiments the enhancers to antimicrobial agents, for example antimicrobial peptides inhibit a gene or gene product. Examples of such gene products are shown in Table 1, and include for example, but are not limited to agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or homologues, variants and fragments thereof.

TABLE 1
List of example genes that can be inhibited.

| SEQ | | | GeneBank | | |
|---|---|---|---|---|---|
| ID NO: | Gene | BLAT_ID | ID | Accession ID | DESCRIPTION |
| 1 | agaA | b3135 | 2367198 | AAC76169 | putative N-acetylgalactosamine-6-phosphate deacetylase |
| 2 | atpA | b3734 | 1790172 | AAC76757 | membrane-bound ATP synthase, F1 sector, alpha-subunit |
| 3 | atpB | b3738 | 1790176 | AAC76761 | membrane-bound ATP synthase, F0 sector, subunit a |
| 4 | atpC | b3731 | 1790169 | AAC76754 | membrane-bound ATP synthase, F1 sector, epsilon-subunit |
| 5 | aptD | b3732 | 1790170 | AAC76755 | Membrane-bound ATP synthase, F1 sector, beta-subunit |
| 6 | atpE | b3737 | 1790175 | AAC76760 | membrane-bound ATP synthase, F0 sector, subunit c |
| 7 | atpG | b3733 | 1790171 | AAC76756 | membrane-bound ATP synthase, F1 sector, gamma-subunit |
| 8 | atpH | b3735 | 1790173 | AAC76758 | membrane-bound ATP synthase, F1 sector, delta-subunit |
| 9 | betB | b0312 | 1786504 | AAC73415 | NAD+-dependent betaine aldehyde dehydrogenase |
| 10 | csdA | b2810 | 1789175 | AAC75852 | orf, hypothetical protein |
| 11 | csdA | b3162 | 1789553 | AAC76196 | inducible ATP-independent RNA helicase |
| 12 | csdB | b1680 | 1787970 | AAC74750 | orf, hypothetical protein |
| 13 | fepC | b0588 | 1786803 | AAC73689 | ATP-binding component of ferric enterobactin transport |
| 14 | guaA | b2507 | 1788854 | AAC75560 | GMP synthetase (glutamine-hydrolyzing) |
| 15 | guaB | b2508 | 1788855 | AAC75561 | IMP dehydrogenase |
| 16 | iscS | b2530 | 1788879 | AAC75583 | putative aminotransferase |
| 17 | kdgK | b3526 | 1789945 | AAC76551 | ketodeoxgluconokinase |
| 18 | lipA | b0628 | 1786846 | AAC73729 | lipoate synthesis, sulfur insertion? |
| 19 | lysA | b2838 | 1789203 | AAC75877 | diaminopimelate decarboxylase |
| 20 | mnmA | | 87081837 | AAC74217.2 | tRNA (5-methylaminomethyl-2-thiouridylate)- |
| | | | | | methyltransferase |
| 21 | nuvC | b2530 | 48994898 | AAT48142 | cysteine desulfurase monomer |
| 22 | papA | b3734 | 1790172 | AAC76757 | membrane-bound ATP synthase, F1 sector, alpha-subunit |
| 23 | pdhx | | 1787926 | AAC74710 | pyridoxine 5′-phosphate oxidase, |
| 24 | phnL | b4096 | 1790534 | AAC77057 | ATP-binding component of phosphonate transport |
| 25 | potE | b0692 | 1786908 | AAC73786 | putrescine transport protein |
| 26 | rpiA | b2914 | 1789280 | AAC75951 | ribosephosphate isomerase, constitutive |
| 27 | sucB | b0727 | 1786946 | AAC73821 | 2-oxoglutarate dehydrogenase (dihydrolipoyltranssuccinase |
| | | | | | E2 component) |
| 28 | trxA | b3781 | 1790215 | AAC76786 | thioredoxin 1 |
| 29 | tusB | b3343 | 1789741 | ACC76368 | tRNA 2-thiouridine synthesizing protein |
| 30 | tusE | b0969 | 87081811 | ACC74055 | tRNA 2-thiouridine synthesizing protein |
| 31 | ubiE | b3833 | 2367307 | AAC76836 | 2-octaprenyl-6-methoxy-1,4-benzoquinone --> 2-octaprenyl- |
| | | | | | 3-methyl-6-methoxy-1,4-benzoquinone |
| 32 | ubiH | b2907 | 1789274 | AAC75945 | 2-octaprenyl-6-methoxyphenol--> 2-octaprenyl-6-methoxy-1, |
| | | | | | 4-benzoquinone |
| 33 | uncA | b3734 | 1710172 | AAC76757 | membrane-bound ATP synthase, F1 sector, alpha-subunit |
| 34 | visB | b2907 | 1789274 | AAC75945 | 2-octaprenyl-6-methoxyphenol--> 2-octaprenyl-6-methoxy-1, |
| | | | | | 4-benzoquinone |
| 35 | yeeY | b2015 | 1788326 | AAC75076 | putative transcriptional regulator LYSR-type |
| 36 | yiaY | b3589 | 1790015 | AAC76613 | putative oxidoreductase |
| 37 | yidK | b3679 | 1790113 | AAC76702 | putative cotransporter |
| 38 | yihV | b3883 | 1790316 | AAD13445 | putative kinase |
| 39 | yfhO | b2530 | 1788879 | AAC75583 | putative aminotransferase |
| 40 | yjbN | b4049 | 1790483 | AAC77019 | orf, hypothetical protein |
| 41 | ynjD | b1756 | 1788053 | AAC74826 | YnjD is an ATP-binding component of a predicted metabolite |
| | | | | | uptake ABC transporter |

Definitions

For the purpose of this specification it will be clearly understood that the word “comprising” means “including but not limited to”, and that the word “comprises” have a corresponding meaning.

As used in this specification the singular forms “a”, “an” and the” include the plural references unless the context clearly dictate otherwise, for example, reference to “an antimicrobial peptide” or “an antimicrobial agent” includes mixtures of antimicrobial peptides or agents respectively, reference to “a antimicrobial agent” includes mixtures of two or more such components, and the like.

The term “microorganism” includes any microscopic organism or taxonomically related macroscopic organism within the categories algae, bacteria, fungi, yeast and protozoa or the like. It includes susceptible and resistant microorganisms, as well as recombinant microorganisms. Examples of infections produced by such microorganisms are provided herein. In one aspect of the invention, the antimicrobial agents and enhancers thereof are used to target microorganisms in order to prevent and/or inhibit their growth, and/or for their use in the treatment and/or prophylaxis of an infection caused by the microorganism, for example multi-drug resistant microorganisms. In some embodiments, gram-negative microorganisms are also targeted.

The anti-pathogenic aspects of the invention target the broader class of “microorganism” as defined herein. However, given that a multi-drug resistant microorganism is so difficult to treat, the antimicrobial agent and an enhancer of antimicrobial agent in the context of the anti-pathogenic aspect of the invention is suited to treating all microorganisms, including for example multi-drug resistant microorganisms.

Unless stated otherwise, in the context of this specification, the use of the term “microorganism” alone is not limited to “multi-drug resistant organism”, and encompasses both drug-susceptible and drug-resistant microorganisms. The term “multi-drug resistant microorganism” refers to those organisms that are, at the very least, resistant to more than two antibiotics in different antibiotic classes. This includes those microorganisms that have more resistance than those that are resistant to three or more antibiotics in a single antibiotic class. This also includes microorganisms that are resistant to a wider range of antibiotics, i.e. microorganisms that are resistant to one or more classes of antibiotics.

The term “microorganism” includes any microscopic organism or taxonomically related macroscopic organism within the categories algae, bacteria, fungi, yeast and protozoa or the like. The microorganisms targeted in the first aspect of the present invention are multi-drug resistant microorganisms. Preferably, gram-negative microorganisms are targeted

The term “antimicrobial agent” as used herein refers to any entity with antimicrobial activity, i.e. the ability to inhibit the growth and/or kill bacterium, for example gram positive- and gram negative bacteria. An antimicrobial agent includes any chemical, peptide, peptidomimetic, entity or moiety, or analogues of hybrids thereof, including without limitation synthetic and naturally occurring non-proteinaceous entities. In some embodiments, the antimicrobial agent is a small molecule having a chemical moiety. For example, chemical moieties include unsubstituted or substituted alkyl, aromatic or heterocyclyl moieties including macrolides, leptomycins and related natural products or analogues thereof. Antimicrobial agents can be any entity known to have a desired activity and/or property, or can be selected from a library of diverse compounds. The term “agent” as used herein and throughout the application is intended to refer to any means such as an organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof.

The term “antimicrobial peptide” as used herein refers to any peptides with antimicrobial activity, i.e. the ability to inhibit the growth and/or kill bacterium, for example gram positive- and gram negative bacteria. The term antimicrobial peptides encompasses all peptides that have antimicrobial activity, and are typically, for example but not limited to, short proteins, generally between 12 and 50 amino acids long, however larger proteins with such as, for example lysozymes are also encompassed as antimicrobial peptides in the present invention. Also includes in the term antimicrobial peptide are antimicrobial peptidomimetics, and analogues or fragments thereof. The term “antimicrobial peptide” also includes all cyclic and non-cyclic antimicrobial peptides, or derivatives or variants thereof, including tautomers, see Li et al. JACS, 2006, 128: 5776-85 and http://aps.unmc.edu/AP/main.php for examples, which are incorporated herein in their entirety by reference. In some embodiments, the antimicrobial peptide is a lipopeptide, and in some embodiments the lipopeptide is a cyclic lipopeptide. The lipopeptides include, for example but not limited to, the polymyxin class of antimicrobial peptides.

The term “polymyxin” is used in its broadest sense to encompass all members of the well known polymyxin class of antibiotics and synthetic derivatives thereof. Derivatives within this class are the non-cyclic derivatives of cyclic polymyxin, derivatives containing amino acid variations, derivatives containing substitutes of the fatty acid components with other fatty acids or substituents, derivatives with D- and L-amino acid conversions, and derivatives substituted with any one or more optional substituents identified below. Classic polymyxins include, but are not limited to, polymyxin A, B1, B2, C, D1, D2, E1 and/or E2, F C, M, P, S and T. The polymyxins are cationic detergents and are relatively simple basic peptides with molecular masses of about 1000-1200 daltons.

In this embodiment, the term “polymyxin resistant” refers to those microorganisms that are resistant to the member of the polymyxin class of lipopeptides being used in one embodiment.

The term “prevent or inhibit growth” of a microorganism, for example a multi-drug-resistant microorganism refers to the interference with growth or replication of the microorganism, which can include but does not necessarily extend to killing of the microorganism.

The term “treatment and/prophylaxis” refers generally to afflicting a subject, tissue or cell to obtain a desired pharmacologic arid/or physiologic effect. The effect may be prophylactic in terms of completely or partially preventing a disease or sign or symptom thereof, and/or may be therapeutic in terms of a partial or complete cure of a disease.

The term “synergy” or “synergistically” are used interchangeably herein refers to the total increase in activity of the antimicrobial agent and the enhancer of antimicrobial agent components of the invention, over their single and/or additive antimicrobial activity. It includes the increase in activity of only one of the antimicrobial components. In the present invention, the antimicrobial agent and enhancer of antimicrobial agent component show at least synergistic anti-pathogenic activity.

The term “bidirectional synergy” refers to the increase in activity of each antimicrobial component (i.e. the antimicrobial agent and/or antimicrobial peptide and enhancer of the agent or enhancer of antimicrobial peptide) when used in conjunction with the other antimicrobial component, and not merely an increase in activity of one of the antimicrobial components. In the present invention, the antimicrobial agent and enhancer of antimicrobial agent component show at least synergistic antimicrobial activity. Advantageously, for example, the antimicrobial agent (for example antimicrobial peptide) and enhancer of the antimicrobial agent (for example antimicrobial peptide) show bidirectional synergistic antimicrobial activity. Stated in other words, for example, if an antimicrobial agent is the antimicrobial peptide colistin, and the enhancer of such antimicrobial peptide is mefloquin, then bidirectional synergy means mefloquin enhances the activity of the antimicrobial peptide colistin and vice versa, the antimicrobial peptide colistin can be used to enhance the activity of mefloquin.

In the present invention, the antimicrobial agent and an enhancer of antimicrobial agent show at least synergistic anti-pathogenic activity. The term “bidirectional synergy” refers to the increase in activity of each antimicrobial component when used in conjunction with the other antimicrobial component, and not merely an increase in activity of one of the antimicrobial components. In the present invention, the antimicrobial agent and enhancer of the antimicrobial agent component show at least synergistic, and in some instances bidirectional synergistic antimicrobial activity.

The term “anti-pathogenic” refers to activity inhibiting or preventing production of a pathogenic factor released by living microorganisms in a host which leads to destructive effects of tissues at the site(s) of infection. This includes inhibition of the expression of flagellin in P. aeruginosa, perturbing of cytokine production and altering action of polymorphonuclear cell functions in vivo and ex vivo, and/or the inhibition of production of pyocyanin, which is produced by microorganisms such as P. aeruginosa and is blue pigment which disrupts human ciliary beating in vitro, inhibits epidermal cell growth and also impedes lymphocyte proliferation.

The term “analog” as used herein refers to a composition that retains the same structure or function (e.g., binding to a receptor) as a polypeptide or nucleic acid herein. Examples of analogs include peptidomimetics, peptide nucleic acids, small and large organic or inorganic compounds, as well as derivatives and variants of a polypeptide or nucleic acid herein. The term “analog” as used herein refers to a composition that retains the same structure or function (e.g., binding to a receptor) as a polypeptide or nucleic acid herein.

The term “derivative” or “variant” as used herein refers to a peptide, chemical or nucleic acid that differs from the naturally occurring polypeptide or nucleic acid by one or more amino acid or nucleic acid deletions, additions, substitutions or side-chain modifications. Amino acid substitutions include alterations in which an amino acid is replaced with a different naturally-occurring or a non-conventional amino acid residue. Such substitutions may be classified as “conservative”, in which case an amino acid residue contained in a polypeptide is replaced with another naturally occurring amino acid of similar character either in relation to polarity, side chain functionality or size.

Substitutions encompassed by the present invention may also be “non conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments amino acid substitutions are conservative.

The term “amino acid” within the scope of the present invention is used in its broadest sense and is meant to include naturally occurring L α-amino acids or residues, but is not necessarily restricted to the naturally occurring amino acids.

By “pharmaceutically acceptable derivative” is meant any pharmaceutically acceptable salt, hydrate or any other compound which, upon administration to the subject, is capable of providing (directly or indirectly) an antimicrobial peptide and/or enhancer of antimicrobial component or residue thereof.

The term “pro-drug” is used herein in its broadest sense to include those compounds and entities which are converted in vivo to active antimicrobial agents and/or active enhancers of antimicrobial agents of the present invention.

The term “tautomer” is used herein in its broadest sense to include antimicrobial agents and/or or enhancers of antimicrobial agents which are capable of existing in a state of equilibrium between two isometric forms. Such compounds may differ in the bond connecting two atoms or groups and the position of these atoms or groups in the compound.

The term “isomer” is used herein in its broadest sense and includes structural, geometric and stereo isomers. As the antimicrobial agents and/or or enhancers of antimicrobial agents may have one or more chiral centers, they are capable of existing in enantiomeric forms.

The term “infection” or “microbial infection” which are used interchangeably herein refers to in its broadest sense, any infection caused by a microorganism and includes bacterial infections, fungal infections, yeast infections and protozomal infections.

Suitable mammals include members of the orders Primates, Rodentla, Lagomorpha, Cetacea, Homo sapiens, Carnivora, Perissodactyla and Artiodactyla. Members of the orders Perissodactyla and Artiodactyla are included in the invention because of their similar biology and economic importance, for example but not limited to many of the economically important and commercially important animals such as goats, sheep, cattle and pigs have very similar biology and share high degrees of genomic homology.

As used herein, the term “effective amount” is meant an amount of antimicrobial agent and/or enhancers of antimicrobial agent components of the present invention effective to yield a desired antibiotic activity. The term “effective amount” as used herein refers to that amount of composition necessary to achieve the indicated effect. The specific “effective amount” will, obviously, vary with such factors as the particular condition being treated, the physical condition of the subject, the type of subject being treated, the duration of the treatment, the route of administration, the type of antimicrobial agent and/or enhancer of antimicrobial agent, the nature of concurrent therapy (if any), and the specific formulations employed, the ratio of the antimicrobial agent and/or enhancers antimicrobial agent components to each other, the structure of each of these components or their derivatives.

As used herein, a “pharmaceutical carrier” is a pharmaceutically acceptable solvent, suspending agent or vehicle for delivering the combination of antimicrobial agent and/or enhancers of antimicrobial agent components to the subject. The carrier may be liquid or solid and is selected with the planned manner of administration in mind. Each carrier must be pharmaceutically “acceptable” in the sense of being compatible with other ingredients of the composition and non injurious to the subject.

The terms “gene(s)” refers to a nucleic acid sequence (DNA, RNA, or analogs and/or combinations thereof) that encodes through its template or messenger RNA a sequence of amino acids characteristic of a specific peptide. The term “gene” can includes intervening, non-coding regions, as well as regulatory regions, and can include 5′ and 3′ ends. Examples of genes associated with, when inactivated, potentiation the effect of antimicrobial agents are, for example but not limited to, agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and etc., and any homologs, analogs or fragments thereof.

The term “gene product(s)” as used herein refers to include RNA transcribed from a gene, or a polypeptide encoded by a gene or translated from RNA.

The term “inhibition” or “inhibit” when referring to the activity of an antimicrobial agent and/or enhancer of antimicrobial agent refers to prevention of, or reduction in the rate of growth. Inhibition and or inhibit when referring to the activity of an enhancer of antimicrobial agent refers to the prevention or reduction of activity of a gene or gene product, that when inactivated potentiates the activity of an antimicrobial agent.

The term “homolog” or “homologous” as used herein refers to homology with respect to structure and/or function. With respect to sequence homology, sequences are homologs if they are at least 50%, preferably at least 60%, more preferably at least 70%, more preferably at least 80%, more preferably at least 90%, more preferably at least 95% identical, more preferably at least 97% identical, or more preferably at least 99% identical. The term “substantially homologous” refers to sequences that are at least 90%, more preferably at least 95% identical, more preferably at least 97% identical, or more preferably at least 99% identical. Homologous sequences can be the same functional gene in different species.

The term “hybridize” refers to interaction of a nucleotide sequence with a second nucleotide sequence. Such interaction can be, e.g., in solution or on a solid support, such as cellulose or nitrocellulose. If a nucleic acid sequence binds to a second nucleotide sequence with high affinity, it is said to “hybridize” to the second nucleotide sequence. The strength of the interaction between the two sequences can be assessed by varying the stringency of the hybridization conditions. Under highly stringent hybridization conditions only highly complementary nucleotide sequences hybridize.

The term “organism” as used herein includes all living cells including microorganisms (e.g., viruses, bacteria, protozoa), plants, and animals (e.g., humans, birds, reptiles, amphibians, fish, and domesticated animals, such as cows, chicken, pigs, dogs, and goats).

The term “peptide” or “polypeptide” are used interchangeably herein, refers to any composition that includes two or more amino acids joined to each other by a peptide bond or peptidomimetic thereof. The term includes both short chains, which are also commonly referred to in the art as peptides, oligopeptides and oligomers, for example, and to longer chains, which generally are referred to in the art as proteins. The term “peptide” includes all peptides as described below. It will be appreciated that peptides often contain amino acids other than the 20 amino acids commonly referred to as the 20 naturally occurring amino acids, and that many amino acids, including the terminal amino acids, can be modified in a given polypeptide, either by natural processes such as glycosylation and other post-translational modifications, or by chemical modification techniques which are well known in the art. Known modifications which can be present in peptides of the present invention include, but are not limited to, acetylation, acylation, ADP-ribosylation, amidation, covalent attachment of flavin, covalent attachment of a heme moiety, covalent attachment of a polynucleotide or polynucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formulation, gamma-c arboxylation, glycation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The term “peptidomimetic” as used herein refers to molecules which mimic an aspect of a polypeptide structure. The term “mimetic” as used herein is any entity, molecule, chemical, small or large molecule, organic or inorganic, synthetic or natural, that mimics the mechanism of the molecule of which it is a mimetic.

The term “purified” refers to a material (e.g., compound, molecule, or structure of interest) that is relatively free of other materials that it normally is associated with and is preferably at least 0.1%, 1%, 10%, 20%, 30%, 40%, 50%, 60%, 70%, 80%, 90%, 91%, 92%, 93%, 94%, 95%, 96%, 97%, 98%, 99% or 100% of total weight of the material.

The term “recombinant” as used herein refers with reference to material (e.g., a cell, a nucleic acid, a protein, or a vector) indicates that such material has been modified by the introduction of a heterologous material (e.g., a cell, a nucleic acid, a protein, or a vector). Thus, for example, recombinant microorganisms or cells express genes that are not found within the native (non-recombinant) form of the microorganism or cell or express native genes that are otherwise abnormally expressed, under expressed or not expressed at all. For example, a recombinant antibody is an antibody which is not normally found in native (non-recombinant) antibodies form.

The term “treatment” or “treating” as used herein refers to reducing or alleviating symptoms in a subject, preventing symptoms from worsening or progressing, or inhibition, elimination, or prevention of the infection, disorder or symptoms in a subject who is free therefrom. “Treating” as used herein covers any treatment of, or prevention of an infection or disease in a vertebrate, a mammal, for example, a human, and includes: (a) preventing the infection and/or disease from occurring in a subject that may be predisposed to the infection and/or disease, but has not yet been diagnosed as having it; (b) inhibiting a infection and/or disease, i.e., arresting its development; or (c) relieving or ameliorating the effects of the infection and/or disease, i.e., cause regression of the effects of the infection or disease.

The term “unit dose” when used in reference to a therapeutic composition of the present invention refers to physically discrete units suitable as unitary dosage for the subject, each unit containing a predetermined quantity of active material calculated to produce the desired therapeutic effect in association with the required physiologically acceptable diluents, i.e., carrier, or vehicle.

The term “subject” as used herein refers to any animal having a disease or condition which requires treatment with a pharmaceutically active agent. The subject may be a mammal, for example a human, or may be a domestic or commercial or companion animal. While in one embodiment of the invention it is contemplated that the antimicrobial agents and/or enhancers of antimicrobial agents of the invention are suitable for use in medical treatment of humans, it is also applicable to veterinary treatment, including treatment of companion animals such as dogs and cats, and domestic animals such as horses, ponies, donkeys, mules, llama, alpaca, pigs, cattle and sheep, or zoo animals such as primates, felids, canids, bovids, and ungulates

Antimicrobial Agents

One aspect of the invention relates to antimicrobial agents. In some embodiments, the antimicrobial agent is antimicrobial peptide. The term “antimicrobial peptide” refers to peptides which have antimicrobial activity. In some embodiments, the antimicrobial agent may be any peptide with antimicrobial activity, for example cyclic and non-cyclic peptides, lipopeptides, and peptides (or proteins) with long chains of amino acids, for example, lyosymes etc. and modified versions thereof. In some embodiments, the antimicrobial peptide is a lipopeptide. The lipopeptides include, for example, the polymyxin class of antimicrobial peptides. The term “polymyxin” is used in its broadest sense to encompass all members of the well known polymyxin class of antibiotics and synthetic derivatives thereof. Derivatives within this class are the non-cyclic derivatives of cyclic polymyxin, derivatives containing amino acid variations, derivatives containing substitutes of the fatty acid components with other fatty acids or substituents, derivatives with D- and L-amino acid conversions, and derivatives substituted with any one or more optional substituents identified below. Classic polymyxins include polymyxin A, B1, B2, C, D1, D2, E1 and/or E2, F C, M, P, S and T. The polymyxins are cationic detergents and are relatively simple basic peptides with molecular masses of about 1000-1200 daltons.

Examples of members of the polymyxin class are polymyxin B (B and B) and polymyxin E (colistin A and B). Both these members include a cyclic heptapeptide ring with a tripeptide side chain. It is envisaged that declyclisation of the ring may result in a peptide with effective antimicrobial activity. Accordingly, cyclic and non-cyclic derivatives and variants of the polymyxins and similar peptides are encompassed within the term “antimicrobial peptide”.

Also included within the scope of “antimicrobial peptide” are all the components of polymyxin B and polymyxin B, as well as synthetic derivatives thereof.

It is envisaged that one or more amino acid sequence(s) of the peptides above can be varied without significant effect on the structure or function of the peptide. Thus, the invention further includes variations of the peptide which show antimicrobial activity such variations or mutants include amino acid deletions, insertions, inversions, repeats and type substitutions.

The structural formula of polymyxin B (B1 and B2) is as follows:

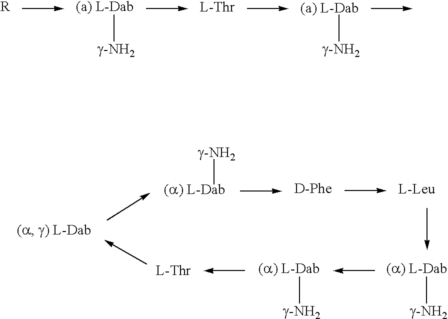

Polymyxin B1: R=(+)-6-methyloctanyl

Polymyxin B2: R=(+)-6-methylheptanyl

Phe: Phenylalanine, Thr: Threonine, Leu: Leucine, Dab=α,γ-diaminobutyric acid, wherein α and γ indicate the respective —NH₂ involved in the peptide linkage.

Colistin

Colistin is a multicomponent polypeptide antibiotic, comprised mainly of colistin A and B, that became available for clinical use in the 1960s, but was replaced in the 1970s by antibiotics considered less toxic. There are two forms of colistin commercially available: colistin sulfate for oral and topical use, and colistimethate sodium (sodium colistin methane sulphonate, colistin sulfomethate sodium) for parenteral use (shown herein); both can be delivered by inhalation. Although there have been a substantial number of clinical reports on the successful use of colistin or polymyxin B (which differs by only one amino acid from colistin) against infections caused by multidrug-resistant P aeruginosa, A baumannii, and K pneumoniae, there is a dearth of information on the clinical pharmacokinetics, pharmacodynamics, and toxicodynamics of colistin; such data are essential for establishing optimal dosing regimens.

Polymyxin B (colistin) has many different components. Its basic structure is a cyclic heptapeptide ring and a tripeptide side chain covalently bound to a fatty acid at the N-terminus via an acyl group. At least 30 components have been isolated and thirteen identified. They differ from each other by the composition of amino acids and fatty acids. The two major components are colistin A and colistin B. The structural formula is as follows:

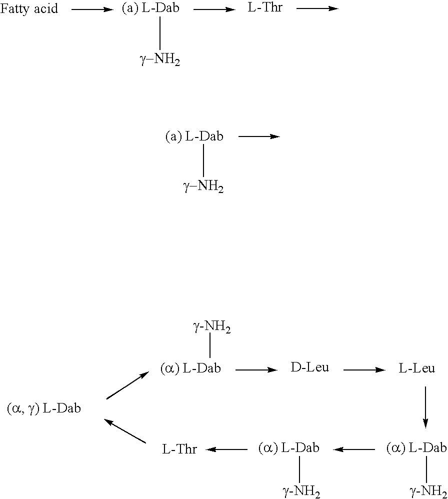

Colistin A: R=methyloctanoic acid

Colistin B: R=6-methylheptanoic acid

Thr: Threonine, Leu: Leucifle, Dab=α,γ-diaminobutyric acid, wherein α and γ indicate the respective —NH₂ involved in the peptide linkage.

Shown below in (A) are structures of Colistin A and Colistin B. Shown in (B) are the structures of colistimethane A and B, fatty acid; 6-methyloctanoic acid for colistin A and 6-methylheptanoic acid for colistin B; Thr-thereonine, Leu-leucine, Dab-α,γ-diaminobutyric acid, α and γ indicate respective amino groups involved in peptide linkage.

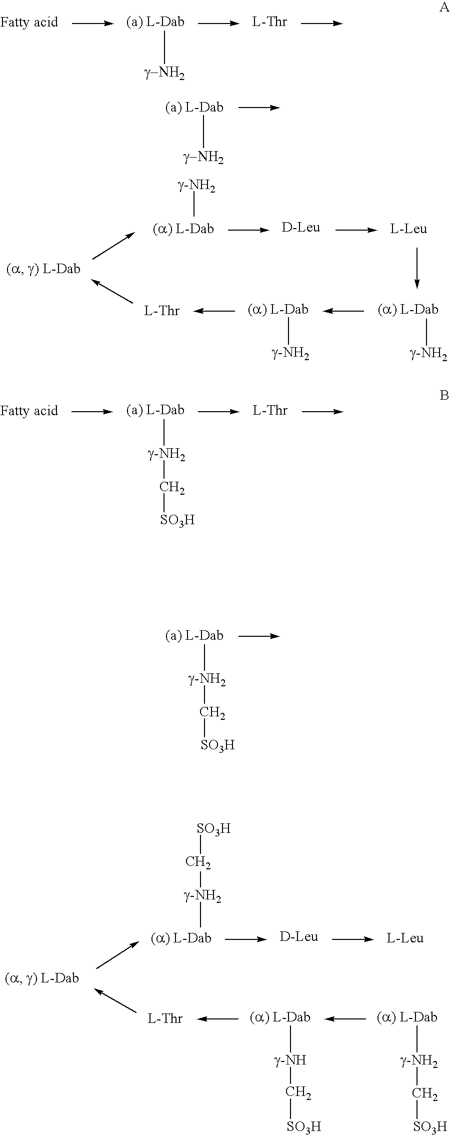

Amino acid substitutions are typically of single residues, but may be of multiple residues, either clustered or dispersed. Additions encompass the addition of one or more naturally occurring or non-conventional amino acid residues. Deletion encompasses the deletion of one or more amino acid residues.

Minor components of colistin include the polymyxin E3 and E4, norvaline-polymyxin E1, valine-polymyxin E1, and valine-polymyxin E2, isoleucine-polymyxin E1, isoleucine-polymyxin E2, polymyxin E7 and isoleucine-polymyxin E0. In some embodiments, the antimicrobial peptide comprises any one or more components of colistin, and in some embodiments, colistin A and/or colistin B. The proportion of colistin A and colistin B in commercial material varies between pharmaceutical suppliers and batches, but it is generally between 4.5:1 to 0.9:1. Colistin is available commercially in two forms, colistin sulphate and sodium colistin methanesulphonate. Sodium colistin methanesulphonate hydrolyses in aqueous media and forms a complex mixture of partially sulphomethylated derivatives plus colistin. One or more of the above forms of colistin or its derivatives are encompassed within the scope of the invention. In some embodiments, antimicrobial peptide comprises salts of colistin, for example salts of pharmaceutically acceptable cations such as sodium, potassium, lithium and the like, acid addition salts of pharmaceutically acceptable inorganic acids such as hydrochloric orthophosphoric, sulphuric and the like, and/or salts of pharmaceutically acceptable organic acids such as acetic, propionic, methanesulfonic and the like.

Although widely used in the literature, the terms colistin and colistimethate are not interchangeable. Colistin (usually used as the sulphate salt) is a polycation, whereas colistimethate (used as the sodium salt) is a polyanion at physiological pH. Colistimethate is prepared from colistin by reaction of the free γ-amino groups of the five α,γ-diaminobutyric acid residues with formaldehyde followed by sodium bisulphite. Colistimethate is not stable in vitro or in vivo, and is hydrolysed to a series of methanesulphonated derivatives plus colistin. Colistin is more stable than colistimethate in human plasma. The differences in chemistry between colistimethate and colistin also translate into differences in pharmacokinetics and pharmacodynamics. Whereas colistimethate is eliminated mainly by the kidney and the urinary excretion involves renal tubular secretion, colistin is eliminated predominantly by the non-renal route because, at least in part, the compound undergoes very extensive renal tubular reabsorption. After intravenous administration of colistimethate (sodium), the plasma half-lives of colistimethate are approximately half of those of the colistin generated from in vivo data. With respect to antibacterial activity against P aeruginosa, recent studies have indicated that colistimethate is a non-active prodrug of colistin. A recent review provides more details on the considerable differences between colistimethate and colistin in their chemistry, pharmacokinetics, and pharmacodynamics.

Another schematic showing the chemical structure of colistin is shown below:

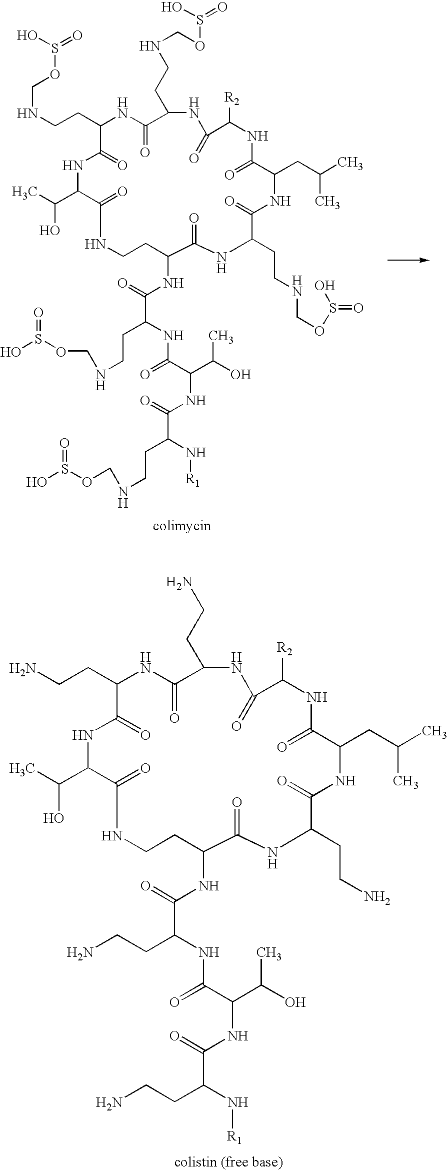

Where R1 and R2 are as follows:

| | R₁ | R₂ |
|---|---|---|
| component I | 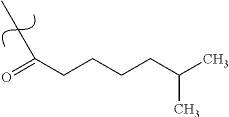 | 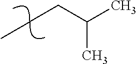 |
| component III | 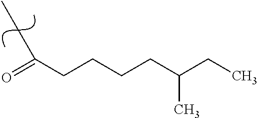 | 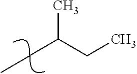 |
| component IV | 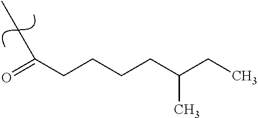 | 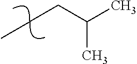 |

Methods to produce pure biologically active colistin, or a pharmaceutically acceptable salt thereof, and methods for its use for treatment for Pseudmonomia aeruginosa, Stenotrophomonas maltophilia and other are disclosed in U.S. Pat. No. 5,767,068 and International Application WO 98/20839 which are incorporated herein in its entirety by reference. In some embodiments, the colistin can be administered with additional agents, as disclosed in International Patent Number WO2006/045156, which is incorporated herein by reference. In particular, International Patent Number WO2006/045156 discloses using colistin with a macrolide component, such as erythromycin, clarithromycin, azithromycin and other components with a lactone ring, where the combination of the macrolide and colistin enhances the anti-pathogenic effects of the macrolide component and the colisitin as compared to their effects alone.

In one embodiment, the antimicrobial peptide comprises colistin methanesulphonate and/or colistin sulphate. In another embodiment, the antimicrobial peptide comprises colistin sulphate.

It is envisaged that variation of these components, for example, by substituting a D-amino acid residue for the same or different L-amino acid residue or vice versa, varying the R substituents and/or conservative amino acid substitutions, while maintaining the synergistic is antimicrobial activity with the peptide enhancer component of the invention, is encompassed within the scope of the invention.

Colisitin is associated with neurotoxicity and nephrotoxicity. The inventors have discovered a dosage regimen and combination of colistin with enhancers of antimicrobial agents as disclosed herein, colistin can be used at reduced doses for the same effect and thereby the inventors have discovered a method whereby colistin can be administered to reduce toxicity effects. Colistin, a cyclic lipopeptide, penetrates the cell wall of G-bacteria by a self induced mechanism by chelating divalent ions, it destabilizes the wall and can insinuate into it. Without being bound by theory, colistin perforates the cell wall, causing distortion of this structure and the release of intracellular constituents in the outside.

Enhancers of Antimicrobial Agents

One aspect of the invention relates to enhancers of antimicrobial agents. In some embodiments, enhancers of antimicrobial agents are inhibitors of the gene products listed in Table 1 or Table 4 as disclosed herein. The inactivation of a gene product by inhibition is considered to potentiate the effectiveness of the antimicrobial agent if the amount of antimicrobial agent used after inactivation is reduced by at least 10% without adversely affecting the result, for example, without adversely effecting the level of antimicrobial activity. In another embodiment, the criteria used to select an enhancers that potentiate the activity of an antimicrobial agent is a reduction of at least . . . 10%, . . . 15%, . . . 20%, . . . 25%, . . . 35%, . . . 50%, . . . 60%, . . . 90% and all amounts in-between of the antimicrobial agent without adversely effecting the antimicrobial effect when compared to the similar cell without the addition of an enhancer of the antimicrobial agent.

In some embodiments, example of such enhancers of antimicrobial agents can include nucleic acids, peptides, nucleic acid analogues, phage, phagemids, polypeptides, peptidomimetics, antibodies, small or large organic or inorganic molecules or any combination of the above. The enhancers of antimicrobial agents can also be naturally occurring or non-naturally occurring (e.g., recombinant) and are sometimes isolated and/or purified.

In some embodiments, where the antimicrobial agent is an antimicrobial peptide, the enhancer is an enhancer to an antimicrobial peptide. In such embodiments, these enhancers are, for example, but not limited to inhibitors of gene products. In some embodiments, the gene products are, for example but not limited to, agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or homologues thereof. In some embodiments, the gene products are, for example but not limited to, those genes listed in Table 4. In some embodiments, the gene products are homologues, variants, fragments or substantially homologous to the following genes; agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or alternatively those listed in Table 4. In other embodiments, the gene products are, for example but not limited to, agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD. The identification of gene products, which when inactivated enhance antimicrobial peptides is discussed in more detail below (see “Screening to Identify Gene Products that suppress the activity of antimicrobial peptides”). Similarly, identification and screening of inhibitors of such gene products is also described in more detail below (see sections titled “Screening For Small Molecules That Inhibit the Gene Product” and “Structure-Based Design Methods to Create Small Molecule enhancers of antimicrobial peptide”).

In some embodiments, enhancers of antimicrobial agents are inhibitors to the gene products listed in table 1, and such inhibitors can be the small molecules as disclosed in Table 2. In some embodiments, an enhancer of the antimicrobial agent as disclosed herein does not include macrolides, such as for example erthyromycin, clarithromycin, azithromyxin, clindamycin, azithromycin and ketolides such as telithromycin. In some embodiments, the enhancer of the antimicrobial agent as disclosed herein does not include the following list of antibiotics; rifampicin, meropenem, ampicillin-sulbactam ciproflozacin, poperacillin-clavulanic acid, imipenem, amikacin, gentamicin and ciproflxicin. While some of these agents may have been used in combination with colistin, their ability to decrease the amount of colistin to be used without decreasing colistin antimicrobial effect as compared to the use of colistin alone cannot be determined because of the limitations in the study design.

ATP synthases: In one embodiment, the gene product, which when inactivated potentiates the activity of antimicrobial agents, is for example an ATP synthase. In some embodiments, the ATP synthase is for example atpA. In alternative embodiments, the gene product is atpA, atpH and/or atpH. ATP synthases is a general term for an enzyme that can synthesize adenosine triphosphate (ATP) from adenosine diphosphate (ADP) and inorganic phosphate by utilizing some form of energy.

An ATP synthase (EC 3.6.3.14) is a general term for an enzyme that can synthesize adenosine triphosphate (ATP) from adenosine diphosphate (ADP) and inorganic phosphate by utilizing some form of energy. ATP syntheses are important in energy metabolism, for example ATP-proton motive force interconversion in the synthesis of energy for use by the cell and/or organism. ATP is formed by proton-conducting, membrane-bound ATP synthase. ATP synthase is a multi-component enzyme complex consisting of two main components: F1 and FO. F1 is on the inner surface of the membrane and is the catalytic center; F1 consists of nine polypeptide chain subunits of five different types. FO is embedded within the membrane and forms the membrane proton channel; in E. coli FO consists of three subunits (A=atpB, B=atpF, C=atpE), F1 consists of five subunits (alpha=atpA, beta=atpD, gamma=atpG, delta=atpH, epsilon=atpC).

ATP synthesis is the fundamental process to provide cell energy and is generated by oxidative phosphorylation. The source of energy is an electrochemical gradient of protons issued from electron transfer across the membranes (Senior et al., 2002). The atp operon of the E. coli ATP syntheses consists of nine genes arranged in the order atpI, atpB, atpE, atpF, atpH, atpA, atpG, atpD, and atpC; similar gene organization have been characterized in other bacteria. Theses gene products assembled into two components: F1 and F0. F1 is on the inner surface of the membrane and is the catalytic center; F1 consists of nine polypeptide chain subunits of five different types. Both the atpA and atpF mutants were primarily identified in the primary screen using the mutant library. The atpF mutant was not retained for further studies. The atpA sub-unit is conserved among bacterial species and mammalians. The level of identity is over 80% for Salmonella sp, Klebsiella pneumo, Yersina, Haemophilus influenza, Yersinia and Vibrio cholera.

Inhibitors of ATP synthases: In some embodiments where the gene product is an ATP synthase, for example atpA, atpH and/or atpH, the inhibitor to the gene product, is an inhibitor to ATP synthases. In some embodiments, the inhibitor of ATP synthases is mefloquine or analogues, mimetics or derivatives thereof. Examples of Mefloquine are the orally administered antimalarial drug used as a prophylaxis against and treatment for malaria, known by the trade name Lariam® (manufactured by Roche Pharmaceuticals) and chemical name mefloquine hydrochloride (formulated with HCI). Mefloquine was developed in the 1970s at the Walter Reed Army Institute of Research in the U.S. as a chemical synthetic similar to quinine. In some embodiments, the ATP synthase inhibitor is Mefloquine hydrochloride, and in some embodiments it is a 4-quinolinemethanol derivative. While Mefloquine is known to have some side effects, its co-administration with an antimicrobial agent, for example an antimicrobial peptide may enable its use at lower therapeutically effective doses, and such, reduce the occurrence of side effects.

In another embodiment, the inhibitor of an ATP synthase can be selected from; venturicidin A, diaryquinoline, Betaine, Acivin, Psicofuranine or derivatives, mimectics or analogues thereof. In alternative embodiments, the inhibitor of ATP synthases is venturicidin or oligomycin or ossamycin or derivatives, mimetics or analogues thereof. The antibiotics venturicidin, oligomycin and ossamycin were investigated as potential inhibitors of the Escherichia coli H+-ATP synthase. It was found that venturicidin strongly inhibited ATP-driven proton transport and ATP hydrolysis, while oligomycin weakly inhibited these functions. Inhibition of the H+-ATP synthase by venturicidin and oligomycin was correlated with inhibition of F0-mediate proton transport. Venturicidin had been shown to be an antifungal of Potential Use in Agriculture.

Any inhibitor of ATP synthase is also encompassed for use in the present invention, including those described herein, as well as those yet unidentified.

Inhibitors of BetB. In another embodiment, the gene product, which when inactivated potentiates the activity of antimicrobial agents, is for example is a Betaine aldehyde dehydrogenase. In some embodiments, the gene product is, for example, a NAD-dependent Betaine aldehyde dehydrogenase. In some embodiments, the Betaine aldehyde dehydrogenase is, for example betB. Bet genes confer protection against osmotic stress by making the osmoprotectant glycine betaine from choline. The bet genes are induced by choline, oxygen, and osmotic stress.

In some embodiments, an inhibitor of an ATP synthases is Betaine. One such inhibitor is, for example but not limited to Cystadane® (betaine anhydrous for oral solution), which is currently used as an agent for the treatment of homocystinuria. In another embodiment, an inhibitor of an ATP synthase which can be used in the methods and compositions as disclosed herein is Acivin, which is an irreversible inhibitor of gamma-glutamyl transpeptidase (ID50=0.54 mM). Inhibits the enzymatic conversion of LTC4 to LTD4. Acivin is currently used as a potent anti-tumor and anti-leishmania agent.

In some embodiments where the gene product is a Betaine aldehyde dehydrogenase, for example betB, the inhibitor to the gene product, is an inhibitor to Betaine aldehyde dehydrogenase. In some embodiments, the inhibitor of Betaine aldehyde dehydrogenase is Betaine or anhydrous betaine or betaine aldehyde chloride or an analogue or derivative or mimetic thereof. Examples of Betaine are Cystadane® (betaine anhydrous for oral solution), which is typically used in the treatment of homocystinuria. Any inhibitor of Betaine aldehyde dehydrogenase is also encompassed for use in the present invention, including those described herein, as well as those yet unidentified.

Inhibitors of GuaA and GuaB: In another embodiment, the gene product, which when inactivated potentiates the activity of antimicrobial agents, is for example is a guanine-monophosphate (GMP) synthase or an inosine-5′-monophosphate (IMP) dehydrogenase or homologues or variants thereof. In some embodiments, the GMP synthase is, for example, guaA and IMP dehydrogenase is, for example, guaB or homologues or variants thereof. GMP synthases and IMP dehydrogenases important in the conversion of IMP to AMP and GMP. These are also used for de novo synthesis as well as in the salvage of purine bases. In some embodiments, the gene products are for example also purA and purB, which are required for the reactions to of IMP to AMP, whereas in other embodiments, the gene products are guaB and guaA, which are required for synthesis of GMP.

In another embodiment, the gene product, which when inactivated potentiates the activity of antimicrobial agents, is for example is a 2-octaprenyl-6-methylphonel hydroxylase, producing 2-octaprenl-6-methyoxy-1,4-benzoquinone and is involved in the electron transport pathway.

In some embodiments where the gene product is a guanine-monophosphate (GMP) synthase, for example guaA or an inosine-5′-monophosphate (IMP) dehydrogenase, for example guaB, the enhancer of the antimicrobial agent is for example Acivin and/or Psicofurine. Acivin is an irreversible inhibitor of gamma-glutamyl transpeptidase (ID₅₀=0.54 mM) and inhibits the enzymatic conversion of LTC4 to LTD4. Typically it is used as a potent anti-tumor and anti-leishmania agent. Psicofuranine was shown to have some antibacterial activities. (Hanka, 1959). Any inhibitor of a guanine-monophosphate (GMP) synthase, for example guaA or an inosine-5′-monophosphate (IMP) dehydrogenase, for example guaB is also encompassed for use in the present invention, including those described herein, as well as those as yet unidentified. Accordingly, in another embodiment, an inhibitor of an ATP synthase which can be used in the methods and compositions as disclosed herein is Psicofuranine, which was shown to have some antibacterial activities. (Hanka, 1959).

Inhibitors of LipA: In another embodiment, the gene product, which when inactivated potentiates the activity of antimicrobial agents, is for example a Lipoyl synthase, or a homologue or variant thereof. An example of a lipoyl synthase is LipA, which is an iron-sulfur protein with SAM-dependent chemistry. Lipoyl synthase is involved in lipoic acid biosynthesis, where the pathway of lipoic acid biosynthesis consists solely of two steps in which two sulfur atoms are introduced into the carbon skeleton at C-8 and C-6. White showed that the sulfur atom in lipoic acid is derived from cysteine. As with the incorporation of the sulfur atom into biotin, the insertion of sulfur in lipoid acid occurs without the formation of either unsaturated or hydroxylated intermediates. Two different sulfur atoms are inserted in lipoic acid biosynthesis, whereas a single sulfur atom inserts into both carbon atoms in biotin biosynthesis. There is evidence to indicate that a in each case, a single enzyme is responsible for the sulfur insertions in each molecule, for example the bioB gene product in biotin biosynthesis and the lipA gene product in lipoic acid biosynthesis. Accordingly, in one embodiment, the enhancer of antimicrobial agent is, for example an inhibitor to Lipoyl synthase, or a homologue or variant thereof. In one embodiment, the inhibitor of Lipoyl synthase, is for example, buthionine sulfoximine or derivatives or modified versions, mimetics or analogues thereof. Any inhibitor of Lipoyl synthase, for example lipA is also encompassed for use in the present invention, including those described herein, as well as those as yet unidentified.

Inhibitors of LysA: In another embodiment, the gene product, which when inactivated potentiates the activity of antimicrobial agents, is for example a diaminopimelate decarboxylase or a homologue or variant thereof. An example of a diaminopimelate decarboxylase is lysA. The expression of diaminopimelate decarboxylase depends on the intracellular concentration of both diaminopimelate, which acts as an inducer, and lysine, which acts as a corepressor. This double regulation reflects the special situation of the diaminopimelate decarboxylase, as a catabolic enzyme for diaminopimelate and an anabolic enzyme for lysine. Moreover, ppGpp must play a role in lysA expression, which is modified in relA mutants. Accordingly, in one embodiment, the enhancer of antimicrobial agent is an inhibitor to diaminopimelate decarboxylase, or homologues or mimetics or variants thereof. In one embodiment, the inhibitor is for example, diaminopimelic acid and/or lysine or analogues, derivatives or homologues thereof. Any inhibitor of a diaminopimelate decarboxylase, for example lysA, is also encompassed for use in the present invention, including those described herein, as well as those as yet unidentified.

Inhibitors of RpiA: In another embodiment, the gene product, which when inactivated potentiates the activity of antimicrobial agents, is for example a ribose-5-phosphate isomerase or a homologue or variant thereof. In some embodiments, the ribose-5-phosphate isomerase is ribose-5-phosphate isomerase A. In some embodiments, the ribose-5-phosphate isomerase A is alkali-inducible. An example of ribose-5-phosphate isomerase A is rpiA. Ribose 5-phosphate isomerases interconvert ribose 5-phosphate and ribulose 5-phosphate. This reaction allows the synthesis of ribose from the pentose phosphate pathway and represents a means for the salvage of carbohydrates after nucleotide breakdown. However, such a dual role for a metabolic pathway is unusual and presents problems for effective metabolic regulation. Two unrelated types of enzymes can catalyze the reaction. RpiA is highly conserved and present in almost all organisms. In E. coli and Salmonella, the enzyme is constitutively expressed. The second type of ribose isomerase, RpiB, is sometimes referred to as AIsB because it can also take part in the metabolism of the rare sugar allose. E. coli strains defective in rpiA are ribose auxotrophs, despite the presence of wild-type rpiB. Ribose prototrophs of an rpiA genetic background arise spontaneously. RpiA exhibits a a/131(a13)/13/a fold, some portions of which are similar to proteins of the alcohol dehydrogenase family. The two subunits of the dimer in the asymmetric unit have different conformations, representing the opening/closing of a cleft. The enzyme presumably acts by an acid-base catalysis mechanism.

Accordingly, in one embodiment, the enhancer of an antimicrobial agent is an inhibitor to ribose-5-phosphate isomerase, for example an inhibitor to ribose-5-phosphate isomerase A, or a homologue or variant thereof. In one embodiment, the inhibitor is for example, 4-phospho-D-erthronhydrixamic acid or analogues, derivatives or homologues or mimetics thereof. Any inhibitor of ribose-5-phosphate isomerases, for example rpiA are also encompassed for use in the present invention, including those described herein, as well as those as yet unidentified.

Inhibitors of TrxA. In another embodiment, the gene product, which when inactivated potentiates the activity of antimicrobial agents, is for example is a Thioredoxin or a homologue or variant thereof. An example of a Thioredoxin is trxA or trxB, which has general chaperone activity, and functions as a processitiviy factor for phage T7 gene 5 DNA polymerase. Accordingly, in one embodiment, an enhancer of antimicrobial agent is an inhibitor to thioredoxin or a variant or homologue thereof. In one embodiment, the inhibitor of thioredoxin is, for example, motexafin gadolinium and/or xycistrin or derivatives, analogues or variants or mimetics thereof. Any inhibitor of thioredoxin, for example an inhibitor of trxA, is also encompassed for use in the present invention, including those described herein, as well as those as yet unidentified.

In some embodiments, the gene product is involved in RNA modifications. Biosynthesis of thionucleoside is a complex process associated with sulfur metabolism and involving numerous proteins including, but not limited to, iscS, tusA, tusE and mnmA.

In another embodiment, the gene product, which when inactivated potentiates the activity of antimicrobial agents, is for example is a ubi or a homologue or variant thereof. An example of a ubi genes is ubiH, which is involved in the ubiquinone synthesis pathway. Ubiquinone is also referred to as coenzyme Q. ubiH is a 2-octaprenyl-6-methoxyphenol hydroxylase and produces 2-octaprenyl-6-methoxy-1,4-benzoquinone. Coenzyme Q is found in the membranes of endoplasmic reticulum, peroxisomes, lysosomes, vesicles and notably the inner membrane of the mitochondrion where it is an important part of the electron transport chain; there it passes reducing equivalents to acceptors such as Coenzyme Q: cytochrome c-oxidoreductase. ubiH gene product is conserved among bacteria showing over 75% identity with the ubiH homologs in Shigella, salmonella and Klebsiella. The level of identity with Pseudomonas is lower at 40%.

Inhibitors of ubiH: ubiH gene which is in the ubiquinone synthesis pathway and the atpA which is a component of the ATP synthase indirectly or directly involved in the electron transfer chain. Ubiquinone is part of a cascade of electron transfer leading to the production of ATP by the ATP synthase. The electron transport chain is the major consumer of oxygen in the cell. The cascade of redox reactions allows the phosphorylation of ADP, forming ATP, utilizing the energy derived from various substrates through the central metabolism (glycolysis, TCA cycle) while reducing NADU or FADH2. The reasons for these genes once inactivated genetically or chemically to potentiate colistin are not clear. It is possible that the membrane potential chain is required to maintain an intact plasma membrane, as the gene products ubiH and atpA are necessary but not essential tough for this process.

It is also encompassed within the present invention that an antimicrobial agent disclosed herein can be combined with any of the inhibitors of the gene products mentioned above, thereby enhancing the activity of antimicrobial agent. As such, inhibitors to the gene products; agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or homologues or variants thereof are referred to as “enhancers of antimicrobial agents” or “enhancers of antimicrobial peptides” herein. In some embodiments, an inhibitor of a gene product is any inhibitor or agent which inhibits the function of a gene listed in Table 4. Such enhancers of antimicrobial agents are for example, but not limited to, mefloquine, venturicidin A, antimycin A, myxothiazol, stigmatellin, diuron, idoacetamide, potassium tellurite hydrate, aDL-vinylglycine, N-Ethylmaleimide, L-Allyglycine, diaryquinoline, betaine aldehyde chloride, acivein, psicofuraine, buthionine sulfoximine, diaminopemelic acid, 4-phospho-D-erythronhydroxamic acid, motexafin gadolinium and/or xycitrin and are summarized in Table 2. In some embodiments, one or more enhancer of antimicrobial agent can be used to enhance or potentiate the activity of antimicrobial agents described herein. Any combination of antimicrobial agent enhancers can be used, in any amount, in any form and by any route of administration. In some embodiments, the enhancers of the antimicrobial agent are administered at the same time or sequentially in any order.

TABLE 2
Examples of enhancers of antimicrobial agents (referred to as “inhibitors”) which inhibit the
genes (or gene products) which, when inactivated, potentiate the effect of antimicrobial agents.

| SEQ ID | | GeneBank | Accession | |
|---|---|---|---|---|
| NO: | Gene | ID | ID | Inhibitor |
| 44 | aceE | | | 2-oxoglutarate or 3-hydroxybutyrate |
| 2 | atpA | 1790172 | AAC76757 | mefloquine, venturicidin A |
| 9 | betB | 1786504 | AAC73415 | betaine aldehyde chloride |
| 45 | folP | | | sulfamethoxazole, Phosphanilic acid |
| 14 | guaA | 1788854 | AAC75560 | acivicin, psicofuranine |
| 15 | guaB | 1788855 | AAC75561 | Tiazofurin, mycophenolic acid |
| 19 | lipA | 1786846 | AAC73729 | buthionine sulfxoximine |
| 20 | lysA | 1789203 | AAC75877 | diaminopimelic acid |
| 46 | nuoJ | | | menaquinone oxidoreductase |
| 27 | rpiA | 1789280 | AAC75951 | 4-phospho-D-erythronohydroxamic acid |
| 47 | sdhC | | | iethylpyrocarbonate, Methylmalonic acid, Myeloperoxidase |
| 29 | trxA | 1790215 | AAC76786 | motexafin gadolinuim, xyctrin |

Inhibitors of Gene Products

In some embodiments, the inhibitors to the gene products which when inactivated, potentiate the effect of antimicrobial agents, include for example antibodies (polyclonal or monoclonal), neutralizing antibodies, antibody fragments, peptides, proteins, peptide-mimetics, aptamers, oligonucleotides, hormones, small molecules, nucleic acids, nucleic acid analogues, carbohydrates or variants thereof that function to inactivate the nucleic acid and/or protein of the gene products identified herein, and those as yet unidentified. Nucleic acids include, for example but not limited to, DNA, RNA, oligonucleotides, peptide nucleic acid (PNA), pseudo-complementary-PNA (pcPNA), locked nucleic acid (LNA), RNAi, microRNAi, siRNA, shRNA etc. The inhibitors can be selected from a group of a chemical, small molecule, chemical entity, nucleic acid sequences, nucleic acid analogues or protein or polypeptide or analogue or fragment thereof. In some embodiments, the nucleic acid is DNA or RNA, and nucleic acid analogues, for example can be PNA, pcPNA and LNA. A nucleic acid may be single or double stranded, and can be selected from a group comprising; nucleic acid encoding a protein of interest, oligonucleotides, PNA, etc. Such nucleic acid sequences include, for example, but not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide inhibitor or fragment thereof, can be, for example, but not limited to mutated proteins; therapeutic proteins and recombinant proteins. Proteins and peptides inhibitors can also include for example; mutated proteins, genetically modified proteins, peptides, synthetic peptides, recombinant proteins, chimeric proteins, antibodies, humanized proteins, humanized antibodies, chimeric antibodies, modified proteins and fragments thereof.

In some embodiments, an enhancer of an antimicrobial agent, such as, but not limited to those listed under inhibitors in Table 2 can be combined with the antimicrobial agent to form one dual mode of action compound. Such methods are well known by person of ordinary skill in the art and include chemical conjugation of two molecules or conjugation by any means known in art. Methods of conjugation of two molecules or entities to form a binary conjugate is described in PCT Patent Application No: WO99/66944, which is specifically incorporated herein in its entirety by reference. In some embodiments, where the enhancer is a polypeptide, such as for example an antibody or peptide inhibitor as disclosed below, methods of conjugation can be, for example, by chemical means, linkers and the like. In some embodiments, the conjugation may be by protein fusion, the methods of which are well know in the art.

In some embodiments, the pharmaceutical composition can comprise a pharmaceutically acceptable carrier and pharmaceutically effective amounts of a microbial agent and an enhancer of an antimicrobial agent which are polypeptide and proteins. In one embodiment, the antimicrobial agent is a colistin, or a homologue or analogue thereof, and in another embodiment, the enhancer of an antimicrobial agent is an inhibitory antibody or polypeptide inhibitor of one of the genes listed in Table 1 or Table 4. In one embodiment, the antimicrobial agent and an enhancer of an antimicrobial agent can be conjugated together, using such methods of protein or polypeptide conjugation which are well known in the art. One can use any method for conjugation of molecules known by persons of ordinary skill in the art, for example, conjugation by chemical means, covalent bonds, linkers and the like. In some embodiments, the conjugation may be protein fusion, the methods of which are well known in the art. For example, BioVertis of Vienna has a dual action compound called Oxaquin, which combines the therapeutic moieties of two different antibiotic compounds into one molecule.

In some embodiments, multi-binding agents are useful in the methods and compositions as disclosed herein, for example multi-binding agents which comprise an antimicrobial agents such as colistin and an enhancer of such antimicrobial agent, such as those inhibiting at least one gene listed in Table 2 or Table 4. Multivalent binding interactions are characterized by the concurrent interaction of multiple ligands with multiple ligand binding sites on one or more cellular receptors. Multivalent interactions differ from collections of individual monovalent interactions by imparting enhanced biological and/or therapeutic effect. Just as multivalent binding can amplify binding affinities; it can also amplify differences in binding affinities, resulting in enhanced binding specificity as well as affinity. An example of a multi-binding agent is an avimer, which relates to a peptide agent which is capable of binding to one or more sites.

Antibodies

In some embodiments, inhibitors of genes and/or gene products useful in the methods of the present invention that function as enhancers to antimicrobial peptides include, for example, antibodies, including monoclonal, chimeric humanized, and recombinant antibodies and fragment thereof. In some embodiments, neutralizing antibodies can be used as inhibitors of the gene products identified herein. Antibodies are readily raised in animals such as rabbits or mice by immunization with the gene product, which when inactivated, potentiate the effect of an antimicrobial agent. Immunized mice are particularly useful for providing sources of B cells for the manufacture of hybridomas, which in turn are cultured to produce large quantities of monoclonal antibodies. Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined. Humanized antibodies are immunoglobin molecules created by genetic engineering techniques in which the murine constant regions are replaced with human counterparts while retaining the murine antigen binding regions. The resulting mouse-human chimeric antibody should have reduced immunogenicity and improved pharmacokinetics in humans. Some examples of high affinity monoclonal antibodies and chimeric derivatives thereof, useful in the methods of the present invention, are described in the European Patent Application EP 186,833; PCT Patent Application WO 92/16553; and U.S. Pat. No. 6,090,923.

In one embodiment of this invention, the inhibitor to the gene products identified herein can be an antibody molecule or the epitope-binding moiety of an antibody molecule and the like. Antibodies provide high binding avidity and unique specificity to a wide range of target antigens and haptens. Monoclonal antibodies useful in the practice of the present invention include whole antibody and fragments thereof and are generated in accordance with conventional techniques, such as hybridoma synthesis, recombinant DNA techniques and protein synthesis. Useful monoclonal antibodies and fragments may be derived from any species (including humans) or may be formed as chimeric proteins which employ sequences from more than one species. Human monoclonal antibodies or “humanized” murine antibody are also used in accordance with the present invention. For example, murine monoclonal antibody may be “humanized” by genetically recombining the nucleotide sequence encoding the murine Fv region (i.e., containing the antigen binding sites) or the complementarily determining regions thereof with the nucleotide sequence encoding a human constant domain region and an Fc region. Humanized targeting moieties are recognized to decrease the immunoreactivity of the antibody or polypeptide in the host recipient, permitting an increase in the half-life and a reduction n the possibly of adverse immune reactions in a manner similar to that disclosed in European Patent Application No. 0,411,893 A2. The murine monoclonal antibodies should preferably be employed in humanized form. Antigen binding activity is determined by the sequences and conformation of the amino acids of the six complementarily determining regions (CDRs) that are located (three each) on the light and heavy chains of the variable portion (Fv) of the antibody. The 25-kDa single-chain Fv (scFv) molecule, composed of a variable region (VL) of the light chain and a variable region (VH) of the heavy chain joined via a short peptide spacer sequence, is the smallest antibody fragment developed to date. Techniques have been developed to display scFv molecules on the surface of filamentous phage that contain the gene for the scFv. scFv molecules with a broad range of antigenic-specificities can be present in a single large pool of scFv-phage library.

One limitation of scFv molecules is their monovalent interaction with target antigen. One of the easiest methods of improving the binding of a scFv to its target antigen is to increase its functional affinity through the creation of a multimer. Association of identical scFv molecules to form diabodies, triabodies and tetrabodies can comprise a number of identical Fv modules. These reagents are therefore multivalent, but monospecific. The association of two different scFv molecules, each comprising a VH and VL domain derived from different parent Ig will form a fully functional bispecific diabody. A unique application of bispecific scFvs is to bind two sites simultaneously on the same target molecule via two (adjacent) surface epitopes. These reagents gain a significant: avidity advantage over a single scFv or Fab fragments. A number of multivalent scFv-based structures has been engineered, including for example, miniantibodies, dimeric miniantibodies, minibodies, (scFv)2, diabodies and triabodies. These molecules span a range of valence (two to four binding sites), size (50 to 120 kDa), flexibility and ease of production. Single chain Fv antibody fragments (scFvs) are predominantly monomeric when the VH and VL domains are joined by, polypeptide linkers of at least 12 residues. The monomer scFv is thermodynamically stable with: linkers of 12 and 25 amino acids length under all conditions. The noncovalent diabody and triabody molecules are easy to engineer and are produced by shortening the peptide linker that connects the variable heavy and variable light chains of a single scFv molecule. The scFv dimers are joined by amphipathic helices that offer a high degree of flexibility and the miniantibody structure can be modified to create a dimeric bispecific (DiBi) miniantibody that contains two miniantibodies (four scFv molecules) connected via a double helix. Gene-fused or disulfide bonded scFv dimers provide an intermediate degree of flexibility and are generated by straightforward cloning techniques adding a C-terminal Gly4Cys sequence. scFv-CH3 minibodies are comprised of two scFv molecules joined to an IgG CH3 domain either directly (LD minibody) or via a very flexible hinge region (Flex minibody). With a molecular weight of approximately 80 kDa, these divalent constructs are capable of significant binding to antigens. The Flex minibody exhibits impressive tumor localization in mice. Bi- and tri-specific multimers can be formed by association of different scFv molecules. Increase in functional affinity can be reached when Fab or single chain Fv antibody fragments (scFv) fragments are complexed into dimers, trimers or larger aggregates. The most important advantage of multivalent scFvs over monovalent scFv and Fab fragments is the gain in functional binding affinity (avidity) to target antigens. High avidity requires that scFv multimers are capable of binding simultaneously to separate target antigens. The gain in functional affinity for scFv diabodies compared to scFv monomers is significant and is seen primarily in reduced off-rates, which result from multiple binding to two or more target antigens and to rebinding when one Fv dissociates. When such scFv molecules associate into multimers, they can be designed with either high avidity to a single target antigen or with multiple specificities to different target antigens. Multiple binding to antigens is dependent on correct alignment and orientation in the Fv modules. For full avidity in multivalent scFvs target, the antigen binding sites must point towards the same direction. If multiple binding is not sterically possible then apparent gains in functional affinity are likely to be due the effect of increased rebinding, which is dependent on diffusion rates and antigen concentration. Antibodies conjugated with moieties that improve their properties are also contemplated for the instant invention. For example, antibody conjugates with PEG that increases their half-life in vivo can be used for the present invention. Immune libraries are prepared by subjecting the genes encoding variable antibody fragments from the B lymphocytes of naive or immunized animals or patients to PCR amplification. Combinations of oligonucleotides which are specific for immunoglobulin genes or for the immunoglobulin gene families are used. Immunoglobulin germ line genes can be used to prepare semisynthetic antibody repertoires, with the complementarily-determining region of the variable fragments being amplified by PCR using degenerate primers. These single-pot libraries have the advantage that antibody fragments against a large number of antigens can be isolated from one single library. The phage-display technique can be used to increase the affinity of antibody fragments, with new libraries being prepared from already existing antibody fragments by random, codon-based or site-directed mutagenesis, by shuffling the chains of individual domains with those of fragments from naive repertoires or by using bacterial mutator strains.

Alternatively, a SCID-hu mouse, for example the model developed by Genpharm, can be used to produce antibodies, or fragments thereof. In one embodiment, a new type of high avidity binding molecule, termed peptabody, created by harnessing the effect of multivalent interaction is contemplated. A short peptide ligand was fused via a semirigid hinge region with the coiled-coil assembly domain of the cartilage oligomeric matrix protein, resulting in a pentameric multivalent binding molecule. In preferred embodiment of this invention, ligands and/or chimeric inhibitors can be targeted to tissue- or tumor-specific targets by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. Alternatively, two or more active agents and or inhibitors attached to targeting moieties can be administered, wherein each conjugate includes a targeting moiety, for example, a different antibody. Each antibody is reactive with a different target site epitope (associated with the same or a different target site antigen). The different antibodies with the agents attached accumulate additively at the desired target site. Antibody-based or non-antibody-based targeting moieties may be employed to deliver a ligand or the inhibitor to a target site. Preferably, a natural binding agent for an unregulated antigen is used for this purpose. For example, diseases such as hepatoma or myeloma are generally characterized by unregulated IL-6 receptors for which IL-6 acts as an autocrine or paracrine moiety with respect to rapid proliferation of these target cell types. For the treatment of such ailments, IL-6 may therefore be employed as a targeting moiety in a targeting protocol of the present invention.

Nucleic Acid Inhibitors

It will be appreciated by those of skill that the genes identified herein and those identified by the methods of the present invention can be readily manipulated to alter the amino acid sequence of a protein. Genes for example, but not limited agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or homologues or variants thereof can be manipulated by a variety of well known techniques for in vitro mutagenesis, among others, to produce variants of the naturally occurring human protein or fragment thereof, herein referred to as muteins, may be used in accordance with the invention.

The variation in primary structure of muteins of agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD are useful in the invention, for instance, may include deletions, additions and substitutions. The substitutions may be conservative or non-conservative. The differences between the natural protein and the mutein generally conserve desired properties, mitigate or eliminate undesired properties and add desired or new properties.

Similarly, techniques for making small oligopeptides and polypeptides that inactivate and/or function as dominant negative versions (i.e. inactive versions) of larger proteins from which they are derived are well known and have become routine in the art. Thus, peptide analogs of gene products of the invention that inactivate the gene product also are useful in the invention.

In some embodiments, RNA interference or “RNAi” can be used as enhancers of antimicrobial agents. In such an embodiment, a RNAi molecule that negatively regulates the expression of the gene products of the invention, for example but not limited to agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or homologues or variants thereof, can be used as enhancers of antimicrobial peptides in the present invention. RNAi is a term initially coined by Fire and co-workers to describe the observation that double-stranded RNA (dsRNA) can block gene expression when it is introduced into worms (Fire et al. (1998) Nature 391, 806-811). dsRNA directs gene-specific, post-transcriptional silencing in many organisms, including vertebrates, and has provided a new tool for studying gene function. RNAi involves mRNA degradation of a target gene. Results showed that RNAi is ATP-dependent yet uncoupled from mRNA translation. That is, protein synthesis is not required for RNAi in vitro. In the RNAi reaction, both strands (sense and antisense) of the dsRNA are processed to small RNA fragments or segments of from about 21 to about 23 nucleotides (nt) in length (RNAs with mobility in sequencing gels that correspond to markers that are 21-23 nt in length, optionally referred to as 21-23 nt RNA). Processing of the dsRNA to the small RNA fragments does not require the targeted mRNA, which demonstrates that the small RNA species is generated by processing of the dsRNA and not as a product of dsRNA-targeted mRNA degradation. The mRNA is cleaved only within the region of identity with the dsRNA. Cleavage occurs at sites 21-23 nucleotides apart, the same interval observed for the dsRNA itself, suggesting that the 21-23 nucleotide fragments from the dsRNA are guiding mRNA cleavage. Isolated RNA molecules (double-stranded; single-stranded) of from about 21 to about 23 nucleotides mediate RNAi. That is, the isolated RNAs mediate degradation of mRNA of a gene to which the mRNA corresponds (mediate degradation of mRNA that is the transcriptional product of the gene, which is also referred to as a target gene). Isolated RNA molecules specific to G6PD mRNA, which mediate RNAi, are antagonists useful in the method of the present invention. Alternative nucleic acid and nucleic acid analogues can be used as enhancers of antimicrobial peptides, for example oligonucleotides, antisense nucleic acid constructs, siRNA, microRNA, shRNA etc.

In some embodiments of the invention suitable enhancers of antimicrobial agents may be administered to the subject in a vector. The vector may be a plasmid vector, a viral vector, or any other suitable vehicle adapted for the insertion and foreign sequence and for the introduction into eukaryotic cells. The vector can be an expression vector capable of directing the transcription of the DNA sequence of the agonist or antagonist nucleic acid molecules into RNA. Viral expression vectors can be selected from a group comprising, for example, retroviruses, lentiviruses, Epstein Barr virus-, bovine papilloma virus, adenovirus- and adeno-associated-based vectors or hybrid virus of any of the above. In one embodiment, the vector is episomal. The use of a suitable episomal vector provides a means of maintaining the agonist or antagonist nucleic acid molecule in the subject in high copy number extra chromosomal DNA thereby eliminating potential effects of chromosomal integration.

Another embodiment of the invention, suitable enhancers of antimicrobial agents can be achieved by introducing catalytic antisense nucleic acid constructs, such as ribozymes, which are capable of cleaving RNA transcripts and thereby preventing the production of wildtype protein. Ribozymes are targeted to and anneal with a particular sequence by virtue of two regions of sequence complementary to the target flanking the ribozyme catalytic site. After binding the ribozyme cleaves the target in a site specific manner. The design and testing of ribozymes which specifically recognize and cleave sequences of the gene products described herein, for example but not limited to agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or homologues or variants thereof can be achieved by techniques well known to those in the art (for example Lleber and Strauss, (1995) Mol Cell Biol 15:540.551, the disclosure of which is incorporated herein by reference).

Uses of Antimicrobial Agents and Enhancers Thereof

Some bacteria, including P. aeruginosa, actively form tightly arranged multi-cell structures in vivo known as biofilm. The production of biofilm is important for the persistence of infectious processes such as seen in pseudomonal lung-infections in patients with cystic fibrosis and diffuse panbronchiolitis and many other diseases. Biofilm is resistant to phagocytosis by host immune cells and the effectiveness of antibiotics at killing bacteria in biofilm structures may be reduced by 10 to 1000 fold. Biofilm production and arrangement is governed by quorum sensing systems. The disruption of the quorum sensing system in bacteria such as P. aeruginosa is an important anti-pathogenic activity as it disrupts the biofilm formation and also inhibits alginate production

The term “microorganism” includes any microscopic organism or taxonomically related macroscopic organism within the categories algae, bacteria, fungi, yeast and protozoa or the like. The microorganisms targeted in the first aspect of the present invention are multi-drug resistant microorganisms. In some embodiments, gram-negative microorganisms are targeted

Bacterial infections include, but are not limited to, infections caused by Bacillus cereus, Bacillus anthracis, Bacillus cereus, Bacillus anthracis, Clostridium botulinum, Clostridium difficle, Clostridium tetani, Clostridium perfringens, Corynebacteria diptheriae, Enterococcus (Streptococcus D), Listeria monocytogenes, Pneumococcal infections (Streptococcus pneumoniae), Staphylococcal infections and Streptococcal infections/Gram-negative bacteria including Bacteroides, Bordetella pertussis, Brucella, Campylobacter infections, enterohaemorrhagic Escherichia coli (EHEC/E. coli 0157:17) enteroinvasive Escherichia coli (EIEC), enterotoxigenic Escherichia coli (ETEC), Haemophilus influenzae, Helicobacter pylori, Klebsiella pneumoniae, Legionella spp., Moraxella catarrhalis, Neisseria gonorrhoeae, Neisseria meningitidis, Proteus spp., Pseudomonas aeruginosa, Salmonella spp., Shigella spp., Vibrio cholera and Yersinia; acid fast bacteria including Mycobacterium tuberculosis, Mycobacterium avium-intracellulars, Mycobacterium johnei, Mycobacterium leprae, atypical bacteria, Chlamydia, Myoplasma, Rickettsia, Spirochetes, Treponema pallidum, Borrelia recurrentis, Borrelia burgdorfii and Leptospira icterohemorrhagiae and other miscellaneous bacteria, including Actinomyces and Nocardia.

In some embodiments, the microbial infection is caused by gram-negative bacterium, for example, P. aeruginosa, A. baumannii, Salmonella spp, Klebsiella pneumonia, Shigella spp. and/or Stenotrophomonas maltophilia.

Examples of microbial infections include bacterial wound infections, mucosal infections, enteric infections, septic conditions, pneumonia, trachoma, onithosis, trichomoniasis and salmonellosis, especially in veterinary practice.

Examples of infections caused by P. aeruginosa include: A) Nosocomial infections; 1. Respiratory tract infections in cystic fibrosis patients and mechanically-ventilated patients; 2. Bacteraemia and sepsis; 3, Wound infections, particularly in burn wound patients; 4. Urinary tract infections; 5. Post-surgery infections on invasive devises 5. Endocarditis by intravenous administration of contaminated drug solutions; 7, Infections in patients with acquired immunodeficiency syndrome, cancer chemotherapy, steroid therapy, hematological malignancies, organ transplantation, renal replacement therapy, and other situations with severe neutropenia.

B) Community-acquired infections; 1. Community-acquired respiratory tract infections; 2. Meningitis; 3. Folliculitis and infections of the ear canal caused by contaminated waters; 4. Malignant otitis externa in the elderly and diabetics; 5. Osteomyelitis of the caleaneus in children; Eye infections commonly associated with contaminated contact lens; 6. Skin infections such as nail infections in people whose hands are frequently exposed to water; 7. Gatrointestinal tract infections; 8. Muscoskeletal system infections.

Examples of infections caused by A. baumannii include: A) Nosocomial infections 1. Bacteraemia and sepsis, 2. respiratory tract infections in mechanically ventilated patients; 3. Post-surgery infections on invasive devices; 4. wound infectious, particularly in bum wound patients; 5. infection in patients with acquired immunodeficiency syndrome, cancer chemotherapy, steroid therapy, hematological malignancies, organ transplantation, renal replacement therapy, and other situations with severe neutropenia; 6. urinary tract infections; 7. Endocarditis by intravenous administration of contaminated drug solutions; 8. Cellulitis.

B) Community-acquired infections; a. community-acquired pulmonary infections; 2. Meningitis; Cheratitis associated with contaminated contact lens; 4. War-zone community-acquired infections.

C) Atypical infections: 1. Chronic gastritis.

Examples of infections caused by Stenotrophomonas maltophilia include Bacteremia, pneumonia, meningitis, wound infections and urinary tract infections. Some hospital breaks are caused by contaminated disinfectant solutions, respiratory devices, monitoring instruments and ice machines. Infections usually occur in debilitated patients with impaired host defense mechanisms.

Examples of infections caused by Klebsiella pneumoniae include community-acquired primary lobar pneumonia, particularly in people with compromised pulmonary function and alcoholics. It also caused wound infections, soft tissue infections and urinary tract infections.

Examples of infections caused by Salmonella app. are acquired by eating contaminated food products. Infections include enteric fever, enteritis and bacteremia.

Examples of infections caused by Shigella spp, include gastroenteristis (shigellosis).

The antimicrobial agent and/or enhancer of antimicrobial agent components of the invention may also be used in various fields as where antiseptic treatment or disinfection of materials it required, for example, surface disinfection.

The antimicrobial agent and enhancers of antimicrobial peptides described herein can be used to treat microorganisms infecting a cell, group of cells, or a multi-cellular organism.

In one embodiment, the antimicrobial agent and enhancers of antimicrobial agent described herein can be used to reduce the rate of proliferation and/or growth of microorganisms. In some embodiments, the microorganism are either or both gram-positive or gram-negative bacteria, whether such bacteria are cocci (spherical), rods, vibrio (comma shaped), or spiral.

Of the cocci bacteria, micrococcus and staphylococcus species are commonly associated with the skin, and Streptococcus species are commonly associated with tooth enamel and contribute to tooth decay. Of the rods family, bacteria Bacillus species produce endospores seen in various stages of development in the photograph and B. cereus cause a relatively mild food poisoning, especially due to reheated fried food. Of the vibrio species, V. cholerae is the most common bacteria and causes cholera, a severe diarrhea disease resulting from a toxin produced by bacterial growth in the gut. Of the spiral bacteria, rhodospirillum and Treponema pallidum are the common species to cause infection (e.g., Treponema pallidum causes syphilis). Spiral bacteria typically grow in shallow anaerobic conditions and can photosynthesize to obtain energy from sunlight.

Moreover, the present invention relates to antimicrobial agent and enhancers of antimicrobial agent that can be used to reduce the rate of growth and/or kill either gram positive, gram negative, or mixed flora bacteria or other microorganisms.

Such bacteria are for example, but are not limited to, listed in Table 3. Further examples of bacteria are, for example but not limited to Baciccis Antracis; Enterococcus faecalis; Corynebacterium; diphtheriae; Escherichia coli; Streptococcus coelicolor; Streptococcus pyogenes; Streptobacillus oniliformis; Streptococcus agalactiae; Streptococcus pneumoniae; Salmonella typhi; Salmonella paratyphi; Salmonella schottmulleri; Salmonella hirshieldii; Staphylococcus epidermidis; Staphylococcus aureus; Klebsiella pneumoniae; Legionella pneumophila, Helicobacter pylori; Mycoplasma pneumonia; Mycobacterium tuberculosis; Mycobacterium leprae; Yersinia enterocolitica, Yersinia pestis; Vibrio cholerae, Vibrio parahaemolyticus; Rickettsia prowozekii; Rickettsia rickettsii; Rickettsia akari; Clostridium difficile; Clostridium tetani, Clostridium perfringens; Clostridianz novyii; Clostridianz septicum; Clostridium botulinum; Legionella pneumophila; Hemophilus influenzue; Hemophilus parainfluenzue; Hemophilus aegyptus; Chlamydia psittaci; Chlamydia trachonZatis; Bordetella pertesis; Shigella spp.; Campylobacter jejuni; Proteus spp.; Citrobacter spp.; Enterobacter spp.; Pseudomonas aeruginosa; Propionibacterium spp.; Bacillus anthracis; Pseudomonas syringae; Spirrilum minus; Neisseria meningitidis; Listeria monocytogenes; Neisseria gonorrhoeae; Treponema pallidum; Francisella tularensis; Brucella spp.; Borrelia recurrentis; Borrelia hermsii; Borrelia turicatue; Borrelia burgdorferi; Mycobacterium avium; Mycobacterium smegmatis; Methicillin-resistant Staphylococcus aureus; Vanomycin-resistant enterococcus; and multi-drug resistant bacteria (e.g., bacteria that are resistant to more than 1, more than 2, more than 3, or more than 4 different drugs).

TABLE 3
Examples of bacteria.

Gram positive bacteria
Staphylococcus aureus
Bacillus anthracis
Bacillus cereus
Bacillus subtilis
Streptococcus pneumonia
Streptococcus pyogenes
Clostridium tetani
Listeria monocytogenes
Mycobacterium tuberculosis
Staphylococcus epidermidis
Gram negative bacteria
Neisseria meningitidis
Neisseria gonorrhoeae
Vibrio cholerae
Escherichia coli K12
Bartonella henselae
Haemophilus influenzae
Salmonella typhi
Shigella dysenteriae
Yersinia pestis
Pseudomonas aeruginosa
Helicobacter pylori
Legionella pneumophila
Others
Borrelia burgdorferi
Ehrlichia chaffeensis
Treponema pallidum
Chlamydia trachomatis

In some embodiments, antimicrobial agent and enhancers of antimicrobial agent described herein is used to treat an already drug resistant bacterial strain such as Methicillin-resistant Staphylococcus aureus (MRSA) or Vancomycin-resistant enterococcus (VRE) of variants thereof.

The present invention also contemplates the use of antimicrobial agent and enhancers of antimicrobial agent described herein in combinations with other antibiotics to fight Gram-positive bacteria that cannot maintain resistance to certain drugs.

As such, antimicrobial agents and enhancers of antimicrobial agents herein may be used to treat infections, for example bacterial infections and other conditions such as urinary tract infections, ear infections, sinus infections, bacterial infections of the skin, bacterial infections of the lungs, sexually transmitted diseases, tuberculosis, pneumonia, lyme disease, and Legionnaire's disease. Thus any of the above conditions and other conditions resulting microorganism infections, for example bacterial infections may be prevented or treated by the compositions of the invention herein.

In another example, an antimicrobial agents and enhancers of antimicrobial agents are used to inhibit resistance to an antiprotozoan agent selected from the group consisting of: Chloroquine; Pyrimethamine; Mefloquine Hydroxychloroquine; Metronidazole; Atovaquone; Imidocarb; Malarone; Febendazole; Metronidazole; Ivomec; Iodoquinol; Diloxanide Furoate; and Ronidazole. Examples of protozoan organisms whose growth is reduced or inhibited by antimicrobial agents and enhancers of antimicrobial agents described herein include but are not limited to, Acanthameba; Actinophrys; Amoeba; Anisonema; Anthophysa; Ascaris lumbricoides; Bicosoeca; Blastocystis hominis; Codonella; Coleps; Cothurina; Cryptosporidia Difflugia; Entamoeba histolytica (a cause of amebiasis and amebic dysentery); Entosipilon; Epalxis; Epistylis; Euglypha; Flukes; Giardia lambia; Hookworm Leishmania spp.; Mayorella; Monosiga; Naegleria Hartmannella; Paruroleptus; Plasmodium spp. (a cause of Malaria) (e.g., Plasmodium falciparum; Plasmodium malariae; Plasmodium vivax and Plasmodium ovale); Pneumocystis carinii (a common cause of pneumonia in immunodeficient persons); microfilariae; Podophya; Raphidiophys; Rhynchomonas; Salpingoeca; Schistosoma japonicum; Schistosoma haematobium; Schistosoma MansOni; Stentor; Strongyloides; Stylonychia; Tapeworms; Trichomonas spp. (e.g., Trichuris trichiuris and Trichomonas vaginalis (a cause of vaginal infection)); Typanosoma spp. and Vorticella.

In another example, antimicrobial agents and enhancers of antimicrobial agents used to inhibit antifungal agent selected from the group consisting of: imidazoles (e. g., clotrimazole, miconazole; econazole, ketonazole, oxiconazole, sulconazole), ciclopiroz, butenafine, and allylamines. Examples of fungus infections growth is reduced or inhibited by antimicrobial agents and enhancers of antimicrobial agents described herein include but are not limited to, tinea; athlete's foot; jock itch; and candida.

Pharmaceutical Formulations.

The present invention contemplates pharmaceutical formulations comprising an antimicrobial agent and an enhancer to such an antimicrobial agent in an effective amount to achieve a therapeutic or prophylactic effect and a pharmaceutically effective carrier.

The actual effective amount will depend upon the condition being treated, the route of administration, the type and class of the antimicrobial agent and enhancer of the antimicrobial agent used to treat the condition, and the medical history of the patient. Determination of the effective amount is well within the capabilities of those skilled in the art. The effective amount for use in humans can be determined from animal models. For example, a dose for humans can be formulated to achieve circulating concentrations that have been found to be effective in animals. The effective amount of an antimicrobial agent and an enhancer to such an antimicrobial agent can vary if the antimicrobial agent and an enhancer of antimicrobial agent is coformulated with another therapeutic agents (for example, antibiotics, antiviral agents, antiprotozoan agents). It is contemplated that lower dosages will be needed in such cases as a result of a synergistic effect of all active ingredients.

In some embodiments, an effective amount of an active ingredient (e.g., an antimicrobial agent and an enhancer of antimicrobial agent and/or additional therapeutic agent(s)) is from about 0.0001 mg to about 500 mg active agent per kilogram body weight of a patient, in some embodiments from about 0.001 to about 250 mg active agent per kilogram body weight of the patient, in further embodiments from about 0.01 mg to about 100 mg active agent per kilogram body weight of the patient, yet still more embodiments from about 0.5 mg to about 50 mg active agent per kilogram body weight of the patient, and in another embodiment from about 1 mg to about 15 mg active agent per kilogram body weight of the patient. In terms of weight percentage, a pharmaceutical formulation of an active agent (e.g., an antimicrobial agent and an enhancer of antimicrobial agent or additional therapeutic agent(s)) can, in some embodiments, comprises of an amount from about 0.0001 wt. % to about 10 wt. %, and in alternative embodiments, from about 0.001 wt. % to about 1 wt. %, and in further embodiments from about 0.01 wt. % to about 0.5 wt. %.

In any of the formulations herein antimicrobial agents and enhancers of antimicrobial agents can be formulated as a salt, a prodrug, or a metabolite. Such formulations can also include additional therapeutic agent(s) such as, for example, antibiotics, antiviral agents, antifungal agents, and/or antiprotozoan agents.

Examples of antibiotics that may be coformulated antimicrobial agents and enhancers of antimicrobial agents include, for example, aminoglycosides, carbapenems, cephalosporins, cephems, glycopeptides, fluoroquinolones/quinolones, oxazolidinones, penicillins, streptogramins, sulfonamides, and tetracyclines.

Aminoglycosides are a group of antibiotics found to be effective against gram-negative. Aminoglycosides are used to treat complicated urinary tract infections, septicemia, peritonitis and other severe intra-abdominal infections, severe pelvic inflammatory disease, endocarditis, mycobacterium infections, neonatal sepsis, and various ocular infections. They are also frequently used in combination with penicillins and cephalosporins to treat both gram-positive and gram-negative bacteria. Examples of aminoglycosides include amikacin, gentamycin, tobramycin, netromycin, streptomycin, kanamycin, paromomycin, and neomycin.

Carbapenems are a class of broad spectrum antibiotics that are used to fight gram-positive, gram-negative, and anaerobic microorganisms. Carbapenems are available for intravenous administration, and as such are used for serious infections which oral drugs are unable to adequately address. For example, carbapenems are often used to treat serious single or mixed bacterial infections, such as lower respiratory tract infections, urinary tract infections, intra-abdominal infections, gynecological and postpartum infections, septicemia, bone and joint infections, skin and skin structure infections, and meningitis. Examples of carbapenems include imipenem/cilastatin sodium, meropenem, ertapenem, and panipenem/betamipron.

Cephalosporins and cephems are broad spectrum antibiotics used to treat gram-positive, gram-negative, and spirochaetal infections. Cephems are considered the next generation Cephalosporins with newer drugs being stronger against gram negative and older drugs better against gram-positive. Cephalosporins and cephems are commonly substituted for penicillin allergies and can be used to treat common urinary tract infections and upper respiratory infections (e.g., pharyugitis and tonsillitis).

Cephalosporins and cephems are also used to treat otitis media, some skin infections, bronchitis, lower respiratory infections (pneumonia), and bone infection (certain; members), and are a preferred antibiotic for surgical prophylaxis. Examples of Cephalosporins include cefixime, cefpodoxime, ceftibuten, cefdinir, cefaclor, cefprozil, loracarbef, cefadroxil, cephalexin, and cephradineze. Examples of cephems include cefepime, cefpirome, cefataxidime pentahydrate, ceftazidime, ceftriaxone, ceftazidime, cefotaxime, cefteram, cefotiam, cefuroxime, cefamandole, cefuroxime axetil, cefotetan, cefazolin sodium, cefazolin, cefalexin.

Fluroquinolones/quinolones are antibiotics used to treat gram-negative infections, though some newer agents have activity against gram-positive bacteria and anaerobes. Fluroquinolones/quinolones are often used to treat conditions such as urinary tract infections, sexually transmitted diseases (e.g., gonorrhea, chlamydial urethritis/cervicitis, pelvic inflammatory disease), gram-negative gastrointestinal infections, soft tissue infections, pphthalmic infections, dermatological infections, sinusitis, and respiratory tract infections (e.g., bronchitis, pneumonia, and tuberculosis). Fluroquinolones/quinolones are used in combination with other antibiotics to treat conditions, such as multi-drug resistant tuberculosis, neutropenic cancer patients with fever, and potentially anthrax. Examples of fluoroquinolones/quinolones include ciproflaxacin, levofloxacin, and ofloxacin, gatifloxacin, norfloxacin, lomefloxacin, trovafloxacin, moxifloxacin, sparfloxacin, gemifloxacin, and pazufloxacin.

Glycopeptides and streptogramins represent antibiotics that are used to treat bacteria that are resistant to other antibiotics, such as methicillin-resistant staphylococcus aureus (MRSA). They are also be used for patients who are allergic to penicillin. Examples of glycopeptides include vancomycin, teicoplanin, and daptomycin.

Carbapenems are used to treat gram-positive, gram-negative, and/or anaerobes.

Oxazolidinones are commonly administered to treat gram-positive infections. Oxazolidinones are commonly used as an alternative to other antibiotic classes for bacteria that have developed resistance. Examples of oxazolidinones include linezolid.

Penicillins are broad spectrum used to treat gram-positive, gram-negative, and spirochaetal infections. Conditions that are often treated with penicillins include pneumococcal and meningococcal meningitis, dermatological infections, ear infections, respiratory infections, urinary tract infections, acute sinusitis, pneumonia, and lyme disease. Examples of penicillins include penicillin, amoxicillin, amoxicillin-clavulanate, ampicillin, ticarcillin, piperacillin-tazobactam, carbenicillin, piperacillin, mezocillin, benzathin penicillin G. penicillin V potassium, methicillin, nafcillin, oxacillin, cloxacillin, and dicloxacillin.

Streptogramins are antibiotics developed in response to bacterial resistance that diminished effectiveness of existing antibiotics. Streptogramins are a very small class of drugs and are currently very expensive. Examples of streptogramins include quinupristin/dafopristin and pristinamycin.

Sulphonamides are broad spectrum antibiotics that have had reduced usage due to increase in bacterial resistance to them. Suphonamides are commonly used to treat recurrent attacks of rheumatic fever, urinary tract infections, prevention of infections of the throat and chest, traveler's diarrhea, whooping cough, meningococcal disease, sexually transmitted diseases, toxoplasmosis, and rhinitis. Examples of sulfonamides include co-trimoxazole, sulfamethoxazole trimethoprim, sulfadiazine, sulfadoxine, and trimethoprim.

Tetracyclines are broad spectrum antibiotics that are often used to treat gram-positive, gram-negative, and/or spirochaetal infections. Tetracyclines are often used to treat mixed infections, such as chronic bronchitis and peritonitis, urinary tract infections, rickets, chlamydia, gonorrhea, lyme disease, and periodontal disease. Tetracyclines are an alternative therapy to penicillin in syphilis treatment and are also used to treat acne and anthrax. Examples of tetracyclines include tetracycline, demeclocycline, minocycline, and doxycycline.

Other antibiotics contemplated herein (some of which may be redundant with the list above) include, but are not limited to; abrifam; acrofloxacin; aptecin, amoxicillin plus clavulonic acid; apalcillin; apramycin; astromicin; arbekacin; aspoxicillin; azidozillin; azlocillin; aztreonam; bacitracin; benzathine penicillin; benzylpenicillin; clarithromycin, carbencillin; cefaclor; cefadroxil; cefalexin; cefamandole; cefaparin; cefatrizine; cefazolin; cefbuperazone; cefcapene; cefdinir; cefditoren; cefepime; cefetamet; cefixime; cefmetazole; cefminox; cefoperazone; ceforanide; cefotaxime; cefotetan; cefotiam; cefoxitin; cefpimizole; cefpiramide; cefpodoxime; cefprozil; cefradine; cefroxadine; cefsulodin; ceftazidime; ceftriaxone; cefuroxime; cephalexin; chloramphenicol; chlortetracycline; ciclacillin; cinoxacin; clemizole penicillin; cleocin, cleocin-T, cloxacillin; corifam; daptomycin; daptomycin; demeclocycline; desquinolone; dibekacin; dicloxacillin; dirithromycin; doxycycline; enoxacin; epicillin; ethambutol; gemifloxacin; fenampicin; finamicina; fleroxacin; flomoxef; flucloxacillin; flumequine; flurithromycin; fosfomycin; fosmidomycin; fusidic acid; gatifloxacin; gemifloxaxin; isepamicin; isoniazid; josamycin; kanamycin; kasugamycin; kitasamycin; kairifam, latamoxef; levofloxacin, levofloxacin; lincomycin; linezolid; lomefloxacin; loracarbaf; lymecycline; mecillinam; methacycline; methicillin; metronidazole; mezlocillin; midecamycin; minocycline; miokamycin; moxifloxacin; nafcillin; nafcillin; nalidixic acid; neomycin; netilmicin; norfloxacin; novobiocin; oflaxacin; oleandomycin; oxacillin; oxolinic acid; oxytetracycline; paromycin; pazufloxacin; pefloxacin; penicillin g; penicillin v; phenethicillin; phenoxymethyl penicillin; pipemidic acid; piperacillin and tazobactam combination; piromidic acid; procaine penicillin; propicillin; pyrimethamine; rifadin; rifabutin; rifamide; rifampin; rifapentene; rifomycin; rimactane, rofact; rokitamycin; rolitetracycline; roxithromycin; rufloxacin; sitafloxacin; sparfloxacin; spectinomycin; spiramycin; sulfadiazine; sulfadoxine; sulfamethoxazole; sisomicin; streptomycin; sulfamethoxazole; sulfisoxazole; quinupristan-dalfopristan; teicoplanin; temocillin; gatifloxacin; tetracycline; tetroxoprim; telithromycin; thiamphenicol; ticarcillin; tigecycline; tobramycin; tosufloxacin; trimethoprin; trimetrexate; trovafloxacin; vancomycin; verdamicin; azithromycin; and linezolid.

A “pharmaceutical acceptable carrier” is a pharmaceutically acceptable solvent, suspending agent or vehicle for delivering the antimicrobial agents and/or an enhancers antimicrobial agents of the present invention to an animal or human. The carrier may be, for example, gaseous, liquid or solid and is selected with the planned manner of administration in mind.

Examples of pharmaceutically acceptable carriers for oral pharmaceutical formulations include: lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. In some embodiments, the carriers are edible oils, for example, corn or canola oils. Polyethylene glycols, e.g. PEG, are also encompassed in the invention as carriers.

Examples of pharmaceutically acceptable carriers for topical formulations include: ointments, cream, suspensions, lotions, powder, solutions, pastes, gels, spray, aerosol or oil. Alternately, a formulation may comprise a transdermal patch or dressing such as a bandage impregnated with active ingredients (e.g., antimicrobial agents and/or an enhancers of antimicrobial agents) and optionally one or more carriers or diluents. The topical formulations may include a compound that enhances absorption or penetration of the active ingredient through the skin or other affected areas. Examples of such dermal penetration enhancers include dimethylsulfoxide and related analogues.

To be administered in the form of a transdermal delivery system, the dosage administration can be continuous rather than intermittent throughout the dosage regimen.

Formulations suitable for parenteral administration include aqueous and non-aqueous formulations isotonic with the blood of the intended recipient; and aqueous and non-aqueous sterile suspensions which may include suspending systems designed to target the compound to blood components or one or more organs. The formulations may be presented in unit-dose or multi-dose sealed containers, for example, ampoules or vials. Extemporaneous injection solutions and suspensions may be prepared from sterile powders, granules and tablets of the kind previously described. Parenteral and intravenous formulation may include minerals and other materials to make them compatible with the type of injection or delivery system chosen.

Commonly used pharmaceutically acceptable carriers for parenteral administration includes, water, a suitable oil, saline, aqueous dextrose (glucose), or related sugar solutions and glycols such as propylene glycol or polyethylene glycols.

Solutions for parenteral administration preferably contain a water soluble salt of the active ingredient, suitable stabilizing agents and, if necessary, buffer substances. Antioxidizing agents, such as sodium bisulfite, sodium sulfite, or ascorbic acid, either alone or combined, are suitable stabilizing agents. Citric acid salts and sodium EDTA may also be used as carriers. In addition, parenteral solutions may contain preservatives, such as benzalkonium chloride, methyl- or propyl-paraben, or chlorobutanol. Suitable pharmaceutical carriers are described in Remington, cited above.

The present invention additionally contemplates antimicrobial agent and enhancers of antimicrobial agents formulated for veterinary administration by methods conventional in the art.

The antimicrobial agents and enhancers of antimicrobial agents described herein can also be formulated for industrial applications with, for example, a cleaning product, such as soap, laundry detergent, shampoo, dishwashing soap, toothpaste, and other house cleaning detergents.

Selection of Subjects Administered the Compositions

In some embodiments, the subjects administered a composition comprising antimicrobial agents and/or enhancers of antimicrobial agents are selected based on the desired treatment regime. For instance,

Accordingly, in some embodiments, subjects are administered the antimicrobial agents, such as colistin, and/or enhancers of antimicrobial agents, such as the inhibitors of the genes as disclosed in Table 1 and Table 4 herein. In some embodiments, an enhancer of an antimicrobial agent can be an antibiotic. In such embodiments, an antibiotic is administered to the subject for the purpose of its gene inhibiting ability, as compared to its normal medical use as an anti-pathogenic or to decrease bacteria viability. Accordingly, the administration of the antibiotics (with the antimicrobial agent) as disclosed herein is different from the normal administration of antibiotics for medical use. As disclosed herein, the administration of an antibiotic is administered according to a treatment regimen which is determined by the desired duration of the treatment or administration with the antimicrobial agent. Accordingly, as disclosed herein, the antibiotic is administered for its gene inhibitory function as compared to its ability to kill bacteria. For example, in general, the medically appropriate administration of an antibiotic is to kill bacteria, and administration is for a specific amount of time which is determined by its ability to kill all the bacteria to a maximum efficacy but to minimize the development of bacterial or viral resistance. As such, antibiotics typically are administered to a subject for a specific period of time, such as, for example, for 5-7 days if used at a high dose, or 10 to 14 days if the antibiotic is used at a lower dose, which results in efficient elimination of the bacteria but does not allow development of bacterial resistance. Extended administration of an antibiotic beyond the time the bacteria is killed results in deleterious results or undesirable side effects. Accordingly, the inventors have discovered that, due to their gene inhibitor functions, antibiotics can be administered to a subjects for a prolonged period of time, which is determined by the duration of administration of the antimicrobial activity, and importantly, not by the antibiotics ability to eliminate bacteria. As such, the antibiotic administration as disclosed herein is counter to the general medical advice on administration of antibiotics to eliminate bacterial or other infections.

Accordingly, in some embodiments, a subject is selected for the administration with the compositions as disclosed herein by identifying a subject that needs a specific treatment regimen of antimicrobial agent, and is administered concurrently an enhancer of a antimicrobial agent such as an antibiotic. As an exemplary example, where the subject is a subject with cystic fibrosis, the subject would be administered a antimicrobial agent to avoid chronic endobronchial infections, such as those caused by pseudomonas aeruginosis or stentrophomonas maltophilia. One such antimicrobial agent is colistin. However, administration of colistin at the doses and the duration required to efficiently prevent such endobronchial infections in subjects is highly toxic and in some instances fatal. Accordingly, in some embodiments of the present invention, the subject is selected for a treatment regimen of an antimicrobial agent, such as colistin, and an enhancer of an antimicrobial agent, such as an antibiotic, and the subject is treated with the compositions as disclosed herein for a specific duration of time. Administration of the compositions as disclosed herein are not directed by the killing of bacteria, but by the need for antimicrobial treatment, and can be, for example more than one week, more than 2 weeks, more than 3 weeks, a month, 2 months, 3 months, 6 months or 12 months or longer.

Importantly, as disclosed herein, the enhancer of the antimicrobial agent as disclosed herein is not selected for its effect on decreasing cell viability, it is selected based on its ability to enhance the effect of the antimicrobial agent. In some embodiments therefore, an enhancer of the antimicrobial agent may not have any anti-pathogenic effects or decrease cell viability when used by themselves, and thus will have no antibiotic or no antimicrobial activity on their own, but when used concurrently with an antimicrobial agent as disclosed herein, such as for example with colistin, the enhancer of the antimicrobial agent functions to enhance the activity of the antimicrobial agent.

Administration

The antimicrobial agents and/or enhancers of antimicrobial agents components may be administered topically, including local delivery to the gastrointestinal tract and other membrane surfaces including aerosol delivery for administration to lungs or nasal cavity, parenterally or orally in dosage unit formulations containing conventional non-toxic pharmaceutically acceptable carriers, adjuvants, and vehicles. The term parenteral as used herein includes subcutaneous injections, intravenous, intramuscular, intrathecal, intraventricular, intracranial injection or infusion techniques. The present invention also provides suitable topical, parenteral and oral pharmaceutical formulations for use in the novel methods of treatment of the present invention.

The compositions and pharmaceutical formulation herein can be administered to an organism by any means known in the art. Routes for administering the compositions and pharmaceutical formulations herein to an animal, such as a human, include parenterally, intravenously, intramuscularly, orally, by inhalation, topically, vaginally, rectally, nasally, buccally, transdermally, or by an implanted reservoir external pump or catheter. When administered to a plan, such means can be by spray or via irrigation.

Although any route of administration may be used, parenteral administration, i.e., administration by injection, is preferred. Injectable formulations can be prepared in conventional forms, either as liquid solutions or suspensions; as solid forms suitable for solubilization or suspension in liquid prior to injection; or as emulsions. Preferably, sterile injectable suspensions are formulated according to techniques known in the art using suitable pharmaceutically acceptable carriers and other optional components as discussed above.

The combination of antimicrobial agents and/or enhancers of antimicrobial agents components may be administered orally as tablets, suspensions, lozenges, troches, powders, granules, emulsions, capsules, syrups or elixirs. The composition for oral use may contain one or more agents selected from the group of sweetening agents, flavoring agents, coloring agents and preserving agents in order to produce pharmaceutically elegant and palatable preparations.

Parenteral administration may be carried out in any number of ways, but it is preferred that the use of a syringe, catheter, or similar device, be used to effect parenteral administration of the formulations described herein. The formulation may be injected systemically such that the active agent travels substantially throughout the entire bloodstream.

In addition, the formulation may also be injected locally to a target site, e.g., injected to a specific portion of the body for which inhibition of mutagenesis is desired. An advantage of local administration via injection is that it limits or avoids exposure of the entire body to the active agent(s) (e.g., antimicrobial peptide and an enhancer of such a peptide and/or other therapeutic agents). It must be noted that in the present context, the term local administration includes regional administration, e.g., administration of a formulation directed to a portion of the body through delivery to a blood vessel serving that body zone. Local delivery may be direct, e.g., intratumoral. Local delivery may also be nearly direct, i.e. intralesional or intraperitoneal, that is, to an area that is sufficiently close to a site of infection so that the active agent(s) exhibit the desired pharmacological activity. Thus, when local delivery is desired, the pharmaceutical formulations are preferably delivered intralesionally, intratumorally, or intraperitoneally.

It is intended that, by local delivery of the presently described pharmaceutical formulations, a higher concentration of the active agent may be directed to the target site. There are several advantages to having high concentrations delivered directly at the target site. First, since the active agent is more localized, there is less potential for toxicity to the patient since minimal systemic exposure occurs. Second, drug efficacy is improved since the target site is exposed to higher concentrations of the drug. Third, relatively fast delivery minimizes solubility and stability liabilities of the active agent before reaching its target site.

Preferably the pharmaceutical compositions are in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet, or it can be the appropriate number of any of these packaged forms.

Useful pharmaceutical dosage formulations for administration of the compounds of the present invention are illustrated as follows:

Capsules: A large number of unit capsules are prepared by filling standard two-piece hard gelatin capsules each with 1-100 milligrams of powdered active ingredient, milligrams of lactose, 50 milligrams of cellulose, and 6 milligrams magnesium stearate.

Soft Gelatin Capsules: A mixture of active ingredient in a digestible oil such as soybean oil, cottonseed oil or olive oil is prepared and injected by means of a positive displacement pump into gelatin to form soft gelatin capsules containing 1-100 milligrams of the active ingredient. The capsules are washed and dried.

Tablets: A large number of tablets are prepared by conventional procedures so that the dosage unit was 1-100 milligrams of active ingredient, 0.2 milligrams of colloidal silicon dioxide, 5-6 milligrams of magnesium stearate, 275 milligrams of microcrystalline cellulose, 11 milligrams of starch and 98.8 milligrams of lactose. Appropriate coatings can be applied to increase palatability or delay absorption.

Injectable: A parenteral composition suitable for administration by injection is prepared by stirring 0.5-1.5% by weight of active ingredient in 10% by volume propylene glycol and water. The solution is made isotonic with sodium chloride and sterilized.

Suspension: An aqueous suspension is prepared for oral administration so that each 5 ml contains 1-100 mg of finely divided active ingredient, 200 mg of sodium carboxymethyl cellulose, 5 mg of sodium benzoate, 1 g of sorbitol solution, U.S.P., and 0.02 ml of vanillin.

Antimicrobial agents and enhancers of antimicrobial agents of the present invention may be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Antimicrobial agents and enhancers of antimicrobial agents of the present invention may be coupled with soluble polymers as targetable drug carriers. Such polymers can include polyvinylpyrrolidone, pyran copolymer, polyhydroxylpropylmethacrylamide-phenol, polyhydroxyethylaspartamidephenol, or polyethyleneoxide-polylysine substituted with palmitoyl residues. Furthermore, the compounds of the present invention can be coupled to a class of biodegradable polymers useful in achieving controlled release of the drug, for example, polylactic acid, polyglycolic acid, copolymers of polylactic and polyglycolic acid, polyepsilon caprolactone, polyhydroxy butyric acid, polyorthoesters, polyacetals, polydibydropyrans, polycyanoacylates, and crosslinked or amphipathic block copolymers of hydrogels.

In some embodiment of this invention, antimicrobial agents and enhancers of antimicrobial agents can be incorporated into a biodistribution directing moiety, such as a polymer, to direct the biodistribution and/or to allow for continuous release of thereof. Alternatively, microparticulate or nanoparticulate polymeric bead dosage forms may be employed. In this case, the antimicrobial agents and/or enhancers of antimicrobial agents will be encapsulated in the particulate dosage forms. In this manner, the antimicrobial agents and/or enhancers of antimicrobial agents are released over time to provide a sustained therapeutic benefit. These sustained release dosage forms are also useful with regard to other active agents useful in the practice of the present invention, such other therapeutic agents discussed below, for example anti-bacterial agents, antibiotics, anti-fungal agents, anti-protozoan agents etc. Release of the active agents (antimicrobial agents and/or enhancers of antimicrobial agents and/or other therapeutic agents) from the particulate dosage forms of the present invention can occur as a result of both diffusion and particulate matrix erosion. Biodegradation rate directly impacts active agent release kinetics.

Controlled release parenteral formulations of the agonists and/or antagonists of the present invention can be made as implants, oily injections, or as particulate systems. Particulate systems include: microspheres, microparticles, microcapsules, nanocapsules, nanospheres, and nanoparticles. Microcapsules contain the therapeutic protein as a central core. In microspheres the therapeutic is dispersed throughout the particle. Liposomes can be used for controlled release as well as drug targeting of entrapped drug.

Antimicrobial agents and enhancers of antimicrobial agents of the present invention can be administered to any organism (eukaryotic or prokaryotic) to prevent or treat drug resistance. Antimicrobial agent and enhancers of such agents can also be administered to a first organism in order to target a second organism associated with the first organism. For example, an antimicrobial agent and enhancers of the antimicrobial agent can be administered to a mammal infected by bacteria or virus or other pathogen, or for example the compositions as disclosed herein can be administered to a plant infected by a fungus or other pathogen. In some embodiments, the methods and compositions as disclosed herein can be used to prevent the development of resistance of a microorganism to the antimicrobial agent, such as colistin. Without being bound by theory, concurrent administration of an enhancer of the microbial agent and an antimicrobial agent as disclosed in the compositions and methods herein, enables targeting of at least two different pathways, such as, for e.g. parallel or downstream pathways, and thus delays or decreases the microorganism's ability to accumulate spontaneous mutations in the genes involved in the pathways targeted by the enhancer of antimicrobial agent and/or antimicrobial agent, and thus decreases the ability of the microorganism to circumvent the ability of the antimicrobial agent to kill the microorganism by spontaneous mutations.

Antimicrobial agents and enhancers of antimicrobial agents of the present invention can be administered as a monotherapy or in combination with additional therapeutic agents (e.g., antibiotic, antiviral, antifungal, antiprotozoan agents etc.) When administered as part of a combination therapy, antimicrobial agents and enhancers of antimicrobial agents herein can be administered serially or simultaneously with the additional agent(s). In some embodiments, antimicrobial agents and enhancers of antimicrobial agents are administered prior to the administration of additional therapeutic agent(s). In other embodiments, antimicrobial agents and enhancers of antimicrobial agents are administered after the administration of additional therapeutic agent(s). For example, for prophylactic benefit, antimicrobial agents and enhancers of antimicrobial agents may be co-administered (concurrent) to a subject at risk of developing an infection, for example a bacterial infection. In some embodiments, the antimicrobial agents and enhancers of antimicrobial agents are administered prior to, or after, the administration of the additional therapeutic agents.

The antimicrobial agent and enhancers of antimicrobial agent components of the present invention may additionally be combined with other medicaments to provide an operative combination. It is intended to include any chemically compatible combination of pharmacologically-active agents, as long as the combination does not eliminate the activity of the antimicrobial peptides and/or macrolide components.

It will be appreciated that the antimicrobial agents and/or enhancers of antimicrobial agents components of the invention and the other medicament may be administered separately, sequentially or simultaneously.

Other medicaments which may be used when treating bacterial infections include salbutamol, ipratropium, dornase alpha, for example, for use in inhalation for respiratory infections such as cystic fibrosis.

Screening to Identify Gene Products that when Inactivated Enhance the Activity of Antimicrobial Agents.

The invention relates to a method of enhancing function of antimicrobial agents by inhibiting (inactivating) the function of a gene product. For example, if a gene product inhibits and/or suppresses the function of an antimicrobial agent and/or enhances the activity of a microorganism, its function decreases the effectiveness of the antimicrobial agent at that dose. Inactivation of specific gene products enhances the effectiveness of the antimicrobial agent. Accordingly, inactivation of gene products enables use of antimicrobial agents for various implications, for example at lower doses, thus reducing possible associated toxicity.

These principles can be used to determine if a given gene product suppresses antimicrobial agent, and thus an inhibitor to such a gene product acts as an enhancer of antimicrobial agent and therefore is a potent candidate in the development of drug to enhance antimicrobial agents.

In one embodiment, a gene product is genetically inactivated using known gene disruption techniques. After such a disruption event, the locus that encoded the gene product would now be unable to produce the gene product and the cell would lack the function of that gene product. Various known ‘mutability’ assays are used to assess the effect of the gene disruption event on a cell's mutability. See Friedberg, E C, Walker, G C, Siede, W. DNA Repair and Mutagenesis (ed. Friedberg, E. C.) American Society of Microbiology, Washington D.C., 1995. For example, an adaptation of the so-called ‘Stressful Lifestyle Associated Mutation’ (or SLAM) assay (wherein the evolution of resistance to an antibiotic of choice is measured) or a forward mutation or reversion assay can be used. See Bull, H J, Lombardo, M J, Rosenberg, S M: Stationary-phase mutation in the bacterial chromosome: recombination protein and DNA polymerase IV dependence. Bull H J., Proc. Natl. Acad. Sci. USA (2001) 98:8334-8341; Friedberg, E C. et al. DNA Repair and Mutagenesis (ea. Friedberg, E. C.) American Society of Microbiology, Washington D.C., 1995; Crouse, G F: Methods (2000) 22:116-119; Rosenberg, S M Nature. Rev. Genet. (2001) 2:504-515; Rosche, W A., Methods (2000) 20:4-17; end roster, PL:, BioEssays (2000) 22:1067-1074.

In one embodiment, bacterial cells with an inactivated gene or a wild-type gene are exposed to one or more antibacterial agent. The number of cells that grow and/or survive in the presence of the antibacterial agent is quantified in both cells with the inactive gene and cells with the wild-type gene. A decrease in the number of cells that grow in the inactivated gene group compared with those with the wild-type gene suggests that the inhibitors to the gene being tested are potential enhancers of antimicrobial agent.

Numerous techniques are known in the art to inactivate genes, many of which could be used to inactivate a test gene of interest. These techniques include the direct inactivation of the test gene, for example via mutation of the test gene via homologous recombination. Another useful technique is the indirect activation of the test gene, for example via mutation of a gene whose gene product modulates the activity of the test gene.

Typically, the test gene is inactivated via one or more mutations such that the resulting protein encoded by the test gene is inactive. Alternatively, the entire gene (or a large portion of the gene's open reading frame) is deleted from the genome. Mutation of the test gene may be carried out using numerous mutagenesis techniques known in the art. At the genetic level, the mutants ordinarily are prepared by site-directed mutagenesis of the DNA encoding the gene. The mutants can be substitution mutants, deletion mutants, or insertion mutants.

In some embodiments, the enhancer to the antimicrobial agent is an inhibitor or a binding agent of the gene product of the following examples of genes: agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yecY, yiaY, yidK, yihV, yfhO, yjbN and/or ynjD. In some embodiments, the genes are homologous or substantially homologous, analogues or variants thereof of agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD, in particular the genes atpA, atpF, atpH, betB, guaA, guaB, lipA, lysA, rpiA and/or trxA. Methods for identifying binding agents are known in the art and include yeast two hybrid systems, etc.

Screening for Small Molecules that Inhibit the Gene Product

Enhancers to antimicrobial agents can be identified by a number of methods including screening libraries of chemical compounds. Combinatorial libraries and methods for searching such libraries are known in the art and include: biological libraries, natural products libraries, spatially addressable parallel solid phase or solution phase libraries, synthetic library methods requiring deconvolution, the ‘one-bead one-compound’ library method, and synthetic library methods using affinity chromatography selection. The biological library approach is largely limited to polypeptide libraries, while the other four approaches are applicable to polypeptide, non-peptide oligomer or small molecule libraries of compounds. See Lam, K. S. (1997) Anticancer Drug Des. 12:145.

In one embodiment, enhancers to antimicrobial agents are screened using Automated Ligand Identification System (referred to herein as “ALIS”). See, e.g., U.S. Pat. Nos. 6,721,665, 6,714,875, 6,694,267, 6,691,046, 6,581,013, 6,207,861, and 6,147,344, which are incorporated herein by reference for all intended purposes. ALIS is a high-throughput technique for the identification of small molecules that bind to proteins of interest (e.g., agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD). Small molecules found to bind tightly to a protein can then be tested for their ability to inhibit the biochemical activity of that protein.

Thus, in some embodiments, a target protein (e.g. atpA, atpF etc) is mixed with pools of small molecules. Preferably, more than 1,000 pools are used, more preferably more than 2,000 pools are used, more preferably more than 3,000 pools are used, or more preferably, more than 10,000 pools are used. Each pool contains approximately, 1,000 compounds, more preferably approximately 2,500 compounds, or more preferably approximately 5,000 compounds that are ‘mass encoded,’ meaning that their precise molecular structure can be determined using only their mass and knowledge of the chemical library.

The small molecules and proteins are mixed together and allowed to come to equilibrium (they are incubated together for 30 minutes at room temperature). The mixture is rapidly cooled to trap bound complexes and subject to rapid size exclusion; chromatography (SEC). Small molecules that bind tightly to the protein of interest will be co-excluded with the protein during SEC. Mass spectroscopic analysis is performed to determine the masses of all small molecules found to bind the protein. Measurement of these masses allows for the rapid determination of the molecular structures of the small molecules.

Compounds that bind to a target gene product (e.g., atpA, atpF etc) in ALIS can then be tested for their ability to inhibit atpA function in vitro. Molecules with potent in vitro inhibitory properties can be tested using a modified Stressful Lifestyle Adaptation and Mutation (referred to herein as “SLAM”) assays, to assess their function as an enhancer of antimicrobial peptide (i.e., the ability to reduce or inhibit the growth and/or survival of E. coli or colistin-resistant E. coli grown on colistin, see Examples). Molecules that function to increase the activity of colistin, for example function as an enhancer of antimicrobial peptide activity in SLAM assays can be selected for further tested.

In one embodiment, a chemical collection of compounds is screened in a format similar to the SLAM assay to identify molecules that function as an enhancer of antimicrobial peptide. Bacterial cells are exposed to either one test compound or a library of compounds and the number of cells that grown over a period of time in the presence of an antimicrobial peptide is determined in the presence and absence of the test compound. A decrease in the number of cells indicates increased inhibition of growth and/or decreased survival of the cells, and thus indicates the test compound or compound functions as an enhancer the antimicrobial peptide. The number of cells is determined both before and after bacteria are exposed to the inhibitor, drug and/or compound. The number of cells is quantified using known assays. See Friedberg, E C, Walker, G C, Siede, W. DNA Repair and Mztagertesis (ea. Friedberg, E. C.) (American Society of Microbiology, Washington D.C., 1995); Crouse, Methods (2000) 22: 116-119.

In yet another embodiment, the bacterial cells are exposed to an antimicrobial agent and the number of cells generated is quantified in the presence and absence of the test compound.

In another example of a method to screen for enhancers of antimicrobial agent, purified gene products, for example agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or homologues or variants thereof are exposed to test compounds. In the presence of the test compounds, inhibitor and/or drug, their function in vitro is assessed, and a reduction in activity identifies a potential enhancer of antimicrobial agent. The inhibition of different gene products described herein by potential enhancers of antimicrobial agent can be quantified using standard methods.

Alternatively, high-throughput assays can be used to screen through large compound libraries to identify potential enhancers of antimicrobial peptides. Such assays rely on arraying the reaction mixtures in 96-well plates, where each well also contains a different enhancer of antimicrobial agents.

Fluorophore labeled nucleoside triphosphates or oligonucleotide primers or templates can be used in conjunction with standard plate handling and visualization procedures to determine which molecules effectively inhibited the activity of a gene product of the invention, for example but not limited to agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or homologues or variants thereof. In one embodiment, libraries can be screened in the presence of one or more of the genes identified, for example agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or homologues or variants thereof, in order to identify drugs, compounds and/or molecules that would most efficiently potentate the effectiveness of an antimicrobial agent synergistically, and thus function as enhancers of antimicrobial agents.

Structure-Based Design Methods to Create Small Molecule Enhancers of Antimicrobial Agents:

In some embodiments, the enhancers of the antimicrobial agents identified herein, and those yet not identified can be modified using molecular modeling software tools to create realistic 3-D models of how molecules are shaped. Such methods include the use of, for example, molecular graphics (i.e., 3D representations) and computational chemistry (e.g., calculations of the physical and chemical properties).

Using such molecular modeling, rational drug design programs can predict which of a collection of different drug like compounds may fit into the active site of an enzyme, and by computationally adjusting their bound conformation, decide which compounds actually might fit the active site well. See William Bains, Biotechnology from A to Z. 2nd edition, Oxford University Press, 1998, at 259.

For basic information on molecular modeling, see, e.g., M. Schlecht, Molecular Modeling on the PC, 1998, John Wiley & Sons; Gans et al., Fundamental Principals of Molecular Modeling, 1996, Plenum Pub. Corp.; N. C. Cohen (editor), Guidebook on Molecular Modeling in Drug Design, 1996, Academic Pres$; and W. B. Smith, Introduction to Theoretical Organic Chemistry and Molecular Modeling, 1996. U.S. Patents which provide detailed information on molecular modeling include U.S. Pat. Nos. 6,093,573; 6,080,576; 5,612,894; 5,583,973; 5,030,103; 4,906,122; and 4,812,12.

The present invention permits the use of molecular and computer modeling techniques to design, and select compounds (e.g., enhancers to antimicrobial agents) that bind and inhibit agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or other gene products that suppress the activity of antimicrobial peptides. Thus, the invention enables, for example, the use of atomic coordinates deposited at the RCSB Protein Data Bank which can be readily identified by persons skilled in the art, to design compounds that interact with such gene products (e.g., agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or homologues or variants thereof). For example, this invention enables the design of compounds that act as competitive inhibitors agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or homologues or variants thereof by binding to, all or a portion of, the active site of these gene products or the gene products listed in Table 4.

This invention also enables the design of compounds that act as uncompetitive inhibitors of agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or homologues or variants thereof. These inhibitors may bind to, all or a portion of, the active site of agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or homologues or variants thereof. Similarly, non-competitive inhibitors; that bind to either agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or homologues or variants thereof (whether or not bound to another chemical entity) may be designed using the atomic coordinates of each gene respectively of this invention.

Alternatively, the atomic coordinates provided by the present invention are useful in designing improved analogues of known inhibitors of the gene products identified herein; for example, but not limited to (e.g., mefloquine, venturicidin A, diayquinoline, betaine aldehyde chloride, acivein, psicofuraine, buthionine sulfoximine, diaminopemelic acid, 4-phospho-D-erythronhydroxamic acid, motexafin gadolinium and/or xycitrin or homologs thereof, and fragments thereof) or to design novel classes of inhibitors. This provides a novel route for designing potent and selective inhibitors.

In alternative embodiments, the present invention also includes the designing of improved analogues of antimicrobial agents, wherein the analogue comprises both the antimicrobial agent and at least one or more enhancer of antimicrobial agents as identified herein. The analogue of the antimicrobial agent and enhancer of antimicrobial agents can be joined by chemical linkage by any means known by persons skilled in the art. In additional embodiments, the antimicrobial agent-enhancer of antimicrobial agent molecule can be subjected to molecule modeling as described above for optimal configuration of the joined molecules.

The availability of both protein crystals and of atomic coordinates determined by X-ray diffraction studies enables ‘soaking’ experiments with agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and etc. crystals with molecules composed of a variety of different chemical entities to identify potential sites for interaction of candidate inhibitors. For example, high resolution X-ray diffraction data collected from crystals saturated with solvent allows the determination of where each type of solvent molecule binds the protein. Small molecules that bind tightly to those sites can then be tested for their ability to inhibit induced mutation (Travis, J., Science (1993) 262: 1374).

Moreover, the present invention enables computational screening of small molecule databases for chemical entities, agents, or compounds that can bind in whole, or in part, to agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or the genes listed in Table 4 and, thereby enhance antimicrobial agent function.

In this screening technique, the quality of fit of such entities or compounds to the binding site may be judged either by shape complementarily or by estimated interaction energy. See Meng, E. C. et al. J. Coma. Chem., 13: 505-524 (1992). The design of compounds that bind to or inhibit agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or the genes listed in Table 4 according to this invention generally involves consideration of two factors. First, the compound must be capable of physically associating with agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc. Inhibition of proteins associated with agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD and/or the genes listed in Table 4 required for their function and/or non-covalent molecular interactions other molecules important in their function, include hydrogen bonding, van der Waals and hydrophobic interactions is also encompassed in this invention. Second, the inhibitor must be able to assume a conformation that allows it to associate with agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc. or other protein required for their function. Although certain portions of the inhibitor will not directly participate in this association with agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc. or associated proteins thereof, those portions may still influence the overall conformation of the molecule. This, in turn, may have a significant impact on potency.

Such conformational requirements include the overall three-dimensional structure and orientation of the chemical entity or compound in relation to all or a portion of the active site of agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc. or other protein required for their function or the spacing between functional groups of a compound comprising several chemical entities that directly interact with agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc., or other protein; required their function.

The potential inhibitory or binding effect of a chemical compound on inhibiting the gene products identified herein, and other potential gene products that suppress the activity of antimicrobial agents may be analyzed prior to its actual synthesis and by the use of computer modeling techniques. If the theoretical structure of the given compound precludes any potential association between it and agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc., or other protein required for their function, synthesis and testing of the compound is obviated.

However, if computer modeling suggests a strong interaction is possible, the molecule may then be synthesized and tested for its ability to interact with a agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc., or other protein required for their function and act as an enhancer of antimicrobial agents of the invention. In this manner, synthesis of inactive compounds may be avoided.

One skilled in the art may use one of several methods to screen chemical entities fragments, compounds, or agents for their ability to associate with agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc., or other protein required for their function and more particularly with the individual binding pockets of such gene products, or associated proteins required for their function. This process may begin by visual inspection of, for example, the active site on the computer screen based on agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc., or other protein required for their function coordinates deposited in the RCSB Protein Data Bank. Selected chemical entities, compounds, or agents may then be positioned in a variety of orientations, or docked, within an individual binding pocket of agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc., or other protein required for their function as defined above. Docking may be accomplished using software such as Quanta and Sybyl, followed by energy minimization and molecular dynamics with standard molecular mechanics force fields, such as CHARMM or AMBER.

Specialized computer programs also assist in the process of selecting chemical entities. These include but are not limited to GRID (Goodford, P. J., J. Med. Chem., (1985) 28, 849-857). GRID is available from Oxford University, Oxford, UK; MCSS (Miranker, A. et al., Structure, Function and Genetics, (1991) Vol. 11, 29-34), MCSS is available from Molecular Simulations, Burlington, Mass., AUTODOCK (Goodsell, D. S. and A. J. Olsen, “Automated Docking of Substrates to Proteins by Simulated Annealing” Proteins: Structure, Function, and Genetics, 8, 195-202 (1990)).

AUTODOCK is available from Scripps Research Institute, La Jolla, Calif.; DOCK (Kuntz, I. D. et al., “A Geometric Approach to Macromolecule-Ligand Interactions” J. Mol. Biol., (1982) 161, 269-288). DOCK is available from University of California, San Francisco, Calif.

Once suitable chemical entities, compounds, or agents have been selected, they can be assembled into a single compound or inhibitor. Assembly may proceed by visual inspection of the relationship of the fragments to each other on the three-dimensional image displayed on a computer screen in relation to the atomic coordinates of agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yecY, yiaY, yidK, yihV, yfhO, yjbN and/or ynjD etc., or other protein required for their function. This would be followed by manual model building using software such as Quanta or Sybyl. Useful programs to aid one of skill in the art in connecting the individual chemical entities, compounds, or agents include but are not limited to CAVEAT (Bartlett, P. A. et al, “CAVEAT: A Program to Facilitate the Structure-Derived Design of Biologically Active Molecules”. In Molecular Recognition in Chemical and Biological Problems”, Special Pub., Royal Chem. Soc., 78, pp. 82-196 (1989)).

CAVEAT is available from the University of California, Berkeley, Calif.; 3D Database systems such as MACCS-3D (MDL Information Systems, San Leandro, Calif.). This area is reviewed in Martin, Y. C., “3D Database Searching in Drug Design”, J. Med. Chem., 35, pp. 2145-2154 (1992); also HOOK (available from Molecular Simulations, Burlington, Mass.).

Instead of designing an inhibitor of atpA, atpF, atpH, betB, guaA, guaB, lipA, lysA, rpiA and/or trxA etc., or other protein required for their in a step-wise fashion one chemical moiety at a time as described above, inhibitors of atpA, atpF, atpH, betB, guaA, guaB, lipA, lysA, rpiA and/or trxA etc., or other protein required for their function may be designed as a whole or “de novo” using either an empty binding site or optionally including some portion(s) of known inhibitor(s). These methods include LUDI (Bohm, H.-J., “The Computer Program LUDI: A New Method for the De Novo Design of Enzyme Inhibitors”, J. ComR. Aid. Molec. Design, (1992) 6, 61-78). LUDI is available from Biosym Technologies, San Diego, Calif. and LEGEND (Nishibata, Y. and A. Itai, Tetrahedron, (1991) 47, p. 8985). LEGEND is available from Molecular Simulations, Burlington, Mass. and LeapFrog (available from Tripos Associates, St. Louis, Mo.).

Other molecular modeling techniques may also be employed in accordance with this invention. See, e.g., Cohen, N. C. et al., “Molecular Modeling Software and; Methods for Medicinal Chemistry,” J. Med. Chem., (1990) 33, 883-894. See also, Navia, M. A. and M. A. Murcko, “The Use of Structural Information in Drug Design”, Current Opinions Structural Biology, (1992) 2, 202-210.

Once a compound has been designed or selected by the above methods, the efficiency with which that compound may bind to agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc., or other protein required for their function may be tested and optimized by computational evaluation. An effective antimicrobial agent and/or enhancer of antimicrobial agent must preferably demonstrate a relatively small difference in energy between its bound and free states (i.e., a small deformation energy of binding). Thus, the most efficient inhibitors of agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc., or other proteins should preferably be designed with deformation energy of binding of not greater than about 10 kcal/mole, or more preferably, not greater than 7 kcal/mole.

Inhibitors of agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc., or other proteins may interact with their target in more than one conformation that is similar in overall binding energy. In those cases, the deformation energy of binding is taken to be the difference between the energy of the free compound and the average energy of the conformations observed when the inhibitor binds to agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD A etc.

A compound designed or selected, as binding to agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc. can be further computationally optimized so that in its bound state it would preferably lack repulsive electrostatic interaction with the target. Such non-complementary (e.g., electrostatic) interactions include repulsive charge-charge, dipole-dipole and charged dipole interactions. Specifically, the sum of all electrostatic interactions between the inhibitor and the enzyme when the inhibitor is bound to its target (e.g., agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD etc. or other protein required for their function), make a neutral or favorable contribution to the enthalpy of binding.

Specific computer software is available in the art to evaluate compound deformation energy and electrostatic interaction. Examples of programs designed for such uses include: Gaussian 92, revision C (M. J. Frisch, Gaussian, Inc., Pittsburgh, Pa., 1992); AMBER, version 4.0 (P. A. Kollman, University of California at San Francisco, 1994); QUANTA/CHARMM (Molecular Simulations, Inc., Burlington, Mass. 1994); and Insight II/Discover (Biosysm Technologies Inc., San Diego, Calif., 1994). These programs may be implemented, for instance, using a Silicon Graphics workstation, IRIS 4D/35 or IBM RISC/6000 workstation model 550. Other hardware systems and software packages will be known to those skilled in the art.

Once an inhibitor of agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN etc. or other proteins that suppress antimicrobial agents has been optimally selected or designed, as described above, substitutions may then be made in some of its atoms or side groups to improve or modify its binding properties. Generally, initial substitutions are conservative, e.g., the replacement group will have approximately the same size, shape, hydrophobicity and charge as the original group. It should, of course, be understood that components known in the art to alter conformation should be avoided. Such substituted chemical compounds may then be analyzed for efficiency of fit into the 3-D structures of agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN etc. or other proteins by the same computer methods described in detail, above.

The compounds designed by any of the above methods are useful for inhibiting genes and/or gene products which when inactivated potentiate activity of antimicrobial agent and thus are useful as therapeutic agents with antimicrobial agents to synergistically inhibit the growth and/or kill microorganisms.

### Examples

The examples presented herein relate to compositions comprising antimicrobial agents and enhancers of antimicrobial agents. In the examples, antimicrobial peptides are used as exemplary antimicrobial agents. Further, colistin is used as an exemplary antimicrobial agent, in particular as an exemplary antimicrobial peptide, although the methods and compositions of the invention are applicable to any antimicrobial agent. Further, mefloquine and potassium tellurite are used as an exemplary enhancer of antimicrobial agents, although the methods and compositions of the invention are applicable to any enhancer of the antimicrobial agent, or any molecule which inhibits a gene listed in Table 1 or Table 4. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Methods

All experiments were performed in Luria-Bertani (LB) medium (Fisher Scientific, Pittsburgh, Pa.). kanamycin (Fisher Scientific), colistin sulfate (Sigma-Aldrich), and potassium tellurite. Kanamycin (50 mg/ml), colistin (25 mg/ml), potassium tellurite (10 mg/ml) stocks on water.

Straind

BW25113 (lacIq rrnBT14 ΔlacZWJ16 hsdR514 ΔaraBADAH33 ΔrhaBADLD78)

Growth of deletion strains ubiH and iscS are contained in a BW25113 deletion library (Baba et al, 2006).

Mutant Deletion Screen

Each plate of knockout strain (11 in total) was first pre-grown in 50 ug/mL of Kanamycin in LB broth, then re-grown in LB broth without Kanamycin.

Twelve 384 well plates were filled with 50 uL LB broth supplemented with 50 ug/mL of Kanamycin in each well. Using a Beckman Multimek pipetting robot. The plates were inoculated from KO library frozen stocks into the newly filled plates using 384 well pin stamper. The stamper was sterilized between each stamps by allowing the pin stamper to sit in 70% Ethanol for 10-15 seconds, briefly placed into 100% Ethanol, then placed over a blame to bum off remaining ethanol. The freshly inoculated plates were incubated for 24 hours at 37 C.

A sample of these re-grown cultures was then diluted 1/250 and stamped onto an LB agar plate. The remaining culture was then treated with colistin [20 ug/mL] for 1.5 Hours. After treatment a second sample is then taken and diluted 1/250 in sterilized H2O before being stamped onto an agar plate in duplicate. After 24 hours of growth at 37° C. the plates are photographed and stored for further image analysis. The digital images of treated vs. non treated plates are automatically overlapped using a developed image analysis program to measure size and density of the resulting colonies.

Colony Formation Unit Assay

For CFU measurements, 50 μl of stationary phase culture was inoculated into 5 ml of LB. 240 or 245 μl of the freshly diluted cells were placed into wells of a 96 well plate (Costar), 5 μl of colistin and or potassium tellurite were added into each well to obtain a final volume of 250 μl. The 96 well plates were then incubated at 37° C. At the 0, 3 and 6 hours time points, 20 μl of culture was collected and then serially diluted in 180 μl of 1×PBS, pH 7.2 (Fisher). A 10 μl portion of each dilution was plated onto LB agar (Fisher)), and the plate was incubated overnight at 37° C. Dilutions that yielded between 20 and 100 colonies were counted and CFU were normalized to reflect the same amount of cells for the starting cultures.

The resistance or susceptibility of microorganism to an antibiotic is classified by using defined minimum inhibitory concentration (MIC) breakpoints. MIC breakpoints for an antibiotic are determined by the MIC distributions of pathogenic in a clinical indication and its pharmacokinetics and pharmacodynamics in humans. While a microorganism may literally be susceptible to a high concentration of an antibiotic in vitro, the microorganism may in fact be resistant to that antibiotic at physiologically realistic concentrations if the concentration of drug required to inhibit growth of or kill, the microorganism is greater than the concentration that can safely be achieved without toxicity Lo the subject, the microorganism is considered to be resistant to the antibiotic. To facilitate the identification of antibiotic resistance or susceptibility using In vitro test results, the National Committee for Clinical Laboratory Standards (NCCLS, now known as Clinical and Laboratory Standards Institute) has formulated standards for antibiotic susceptibility that correlate clinical outcome to in vitro determinations of the MIC antibiotics.

Generally, MIC values indicate resistance or susceptibility of a microorganism. For example, MIC valves of a microorganism to colistin/polymyxin B are as follows: MIC<4 mg/L=susceptible, MIC≧8 mg mg/L=resistant and MIC≧128 mg/L=highly resistant

### Example 1

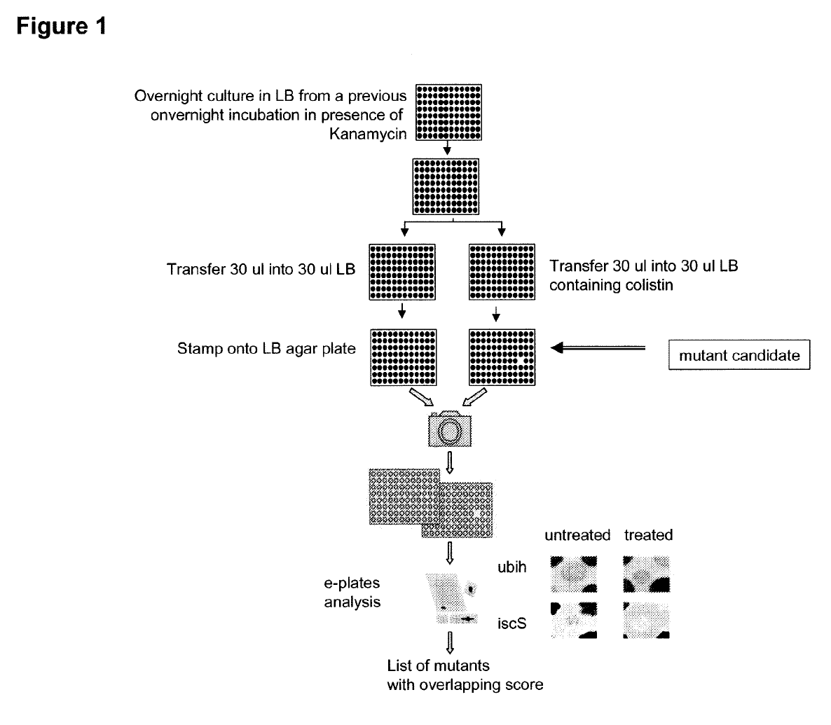

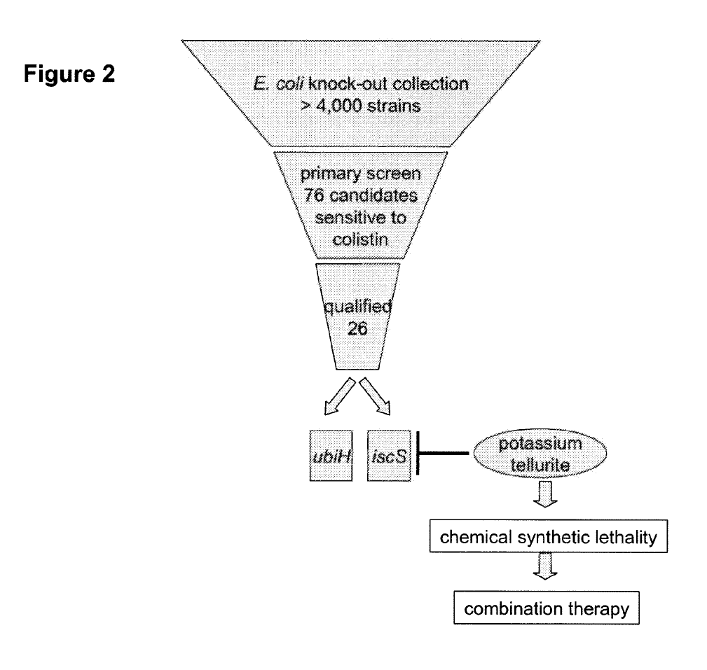

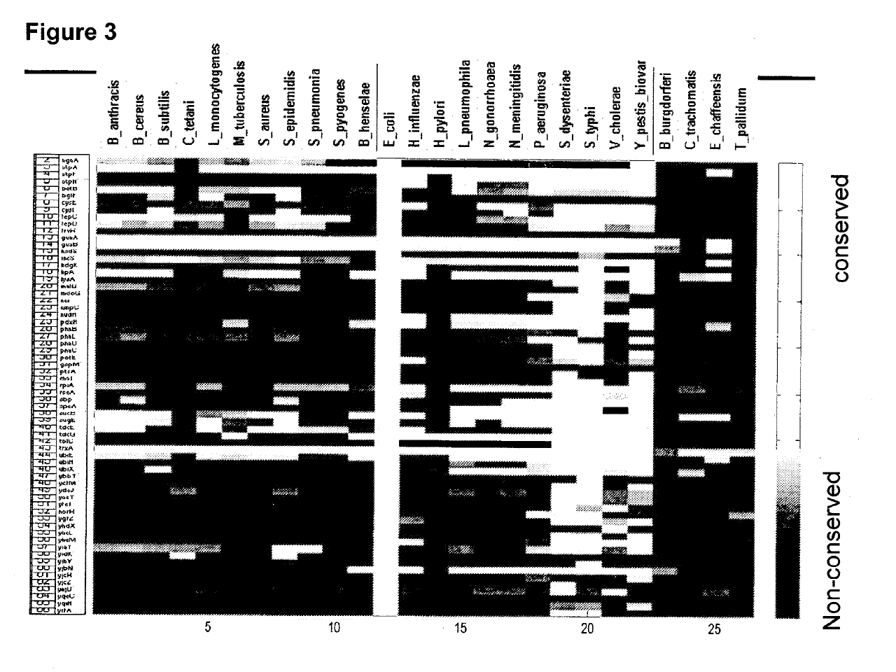

Over 4,000 single mutant E. coli were initially screened in a cell based assay for ability to grow and/or survive in the presence of colistin, an antimicrobial peptide (FIG. 1). Approximately 92-95 E. coli mutants were unable to grow and survive in the presence of colistin. The gene loci of the mutations were analyzed and identified which are listed in FIG. 2. The homology of the identified gene loci (herein referred to as “gene products”) was analyzed with respect to gene homology across different species of bacteria (FIG. 3), and it was found that 24 of 66 gene products had human homologues, listed in Table 1. Of these 24 gene products, 7 had known identified inhibitor which are listed in Table 2.

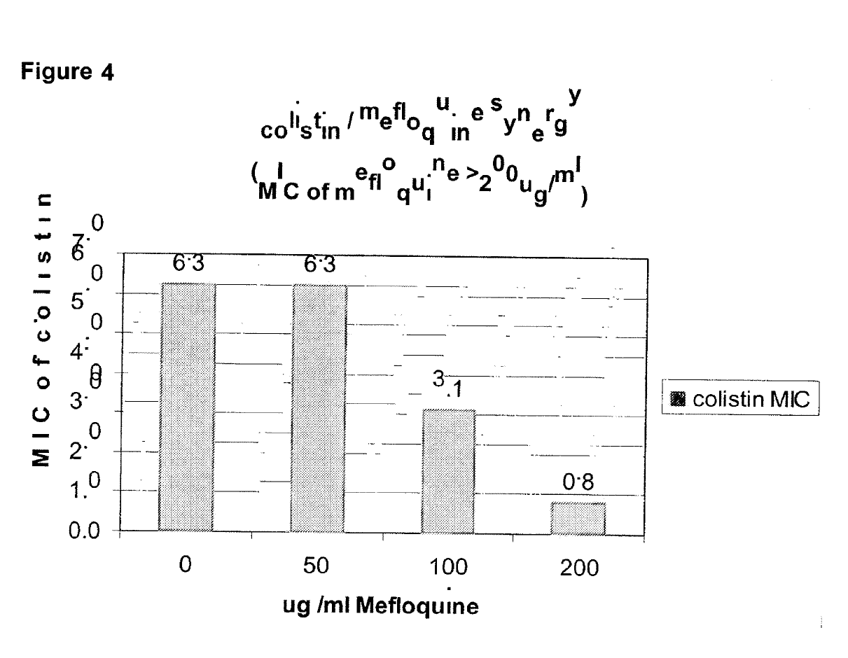

The efficacy of colistin in inhibiting the growth and/or suppressing survival of E. coli in the presence of one inhibitor, mefloquine was assessed in a minimum inhibitory concentration (MIC) assay. As shown in FIG. 4, in the absence of mefloquine, the MIC of colistin is 6.3, whereas in the presence of 50, 100 and 200 μg/ml of mefloquine, the MIC of colistin required to suppress growth is 6.3, 3.1 and 0.8 respectively. Therefore, there is a dose-dependent potentiation of colistin efficacy by mefloquine at the concentrations above 50 μg/ml or greater. Thus, mefloquine was demonstrated to act as a potent enhancer to the antimicrobial peptide colistin. The inventors have also demonstrated that mefloquinine when used concurrently with colistin, potentiates colistin ability to decrease gram-positive bacteria (data not shown).

Accordingly, the inventors have demonstrated that mefloquine is an exemplary example of an enhancer of an antimicrobial agent, and was demonstrated to potentiate the activity of an antimicrobial agent such as colistin. Accordingly, the inventors have discovered that dose and use of mefloquine as an enhancer of the antimicrobial agent is determined by the therapeutic regimen (i.e. duration and administration) of the antimicrobial agent such as colistin, as apposed to any ability to decrease cell viability that mefloquine may have when used by itself.

### Example 2

The inventors used a systems biology platform to identify of several genes and therefore pathway, which once inactivated can increase the killing effect of colistin. This approach has its origin with the notion of synthetic lethality screen in yeast where two genes non essential for viability on their own are found to induce a non viable strain when combined and under specific growth condition(s) (Cottarel, 1997). With the access to the yeast deletion knock out library (reviewed in Ooi et al., 2006) major efforts have been initiated to create genetic mapping of gene interaction (Tong et al., 2004). In a further step such reagents were used to link bioactive compounds to genetic pathways (Parsdons et al., 2004). Reminiscent to this work, our screen was designed to identify mutants exhibiting hyper sensitivity to antibacterials to (i) map a chemogenomic response pathway and (ii) to further identify and/or develop inhibitors which can mimic the genetic observation. Indeed, such gene products can ultimately be the target of compounds which can in turn chemically potentiate Colistin.

The inventors have identified herein using a systems biology platform, several genes and pathways, which once inactivated, can increase the killing effect of colistin. For example, the inventors have discovered a number of genes, such as those listed in Table 1 or Table 4, which normally function in either secondary, parallel or downstream pathways to the target action of the antimicrobial agent, and when such pathways are inhibited or inactivated by inhibitors, potentiate the activity of antimicrobial agents, such as for example colistin. The inventors have discovered, using an assay or screen designed to identify mutants exhibiting hyper sensitivity to antibacterials to (i) map a chemogenomic response pathway and (ii) to further identify and/or develop inhibitors which can mimic the genetic phenotype. Indeed, such gene products can ultimately be the target of compounds which can in turn chemically potentiate colistin.

The Keio collection of E. coli mutants consist of 3,985 single gene knock-out of non essential for viability genes (Baba et al., 2006). In the goal to identify mutants which exhibit increased sensitivity to antibiotics, colistin in that case, the inventors developed a high-throughout assay which the screen basic scheme is shown on FIG. 1. The collection was re-formatted in a 384 well plate, from a 96 well plate format, for ease of manipulation. Each identified candidates found more than once in separated screens were called positives. 73 Positives were then reformatted into a 96 well plate for further testing (FIG. 2, Table 4). From these screen we selected the iscS (Lauhon, 2002) and the ubiH (Young et al, 1973) mutants for further studies.

Such inhibitors of the gene products as disclosed herein are termed “enhancers of antimicrobial agents” herein, and are selected based on their ability to inhibit a gene, such as any of those selected from the list of genes in Table 1 or Table 4. In particular, an inhibitor or enhancer of antimicrobial agent is selected based on its gene inhibiting function rather than any ability it may have to decrease cell viability when it is used by itself.

TABLE 4
List of genes identified to exhibit sensitivity
to colistin from the primary screen.

| Gene | Function |
|---|---|
| agaA | putative N-acetylgalactosamine-6-phosphate deacetylase |
| atpA | membrane-bound ATP synthase, F1 sector, alpha-subunit |
| atpF | membrane-bound ATP synthase, F0 sector, subunit b |
| atpH | membrane-bound ATP synthase, F1 sector, delta-subunit |
| betB | NAD+-dependent betaine aldehyde dehydrogenase |
| bglF | PTS system beta-glucosides, enzyme II, cryptic |
| cysE | serine acetyltransferase |
| cysI | sulfite reductase, alpha subunit |
| fepC | ATP-binding component of ferric enterobactin transport |
| fepD | ferric enterobactin (enterochelin) transport |
| frvR | putative frv operon regulatory protein |
| guaA | GMP synthetase (glutamine-hydrolyzing) |
| guaB | IMP dehydrogenase |
| hofF | putative general protein secretion protein |
| hsdS | specificity determinant for hsdM and hsdR |
| iscS | putative aminotransferase |
| JW4016 | |
| JW5075 | |
| JW5227 | |
| JW5257 | |
| JW5360 | |
| kdgK | ketodeoxygluconokinase |
| lipA | lipoate synthesis, sulfur insertion? |
| lysA | diaminopimelate decarboxylase |
| malG | part of maltose permease, inner membrane |
| mbhA | putative motility protein |
| mdoG | periplasmic glucans biosynthesis protein |
| nei | endonuclease VIII and DNA N-glycosylase with an AP lyase |
| | activity |
| nmpC | outer membrane porin protein; locus of qsr prophage |
| nudH | |
| pdxH | pyridoxinephosphate oxidase |
| phnB | orf, hypothetical protein |
| phnL | ATP-binding component of phosphonate transport |
| phnO | putative regulator, phn operon |
| pnuC | required for NMN transport |
| potE | putrescine transport protein |
| pshM | putative general secretion |
| ptsA | PEP-protein phosphotransferase system enzyme 1 |
| rhaT | rhamnose transport |
| rpiA | ribosephosphate isomerase, constitutive |
| rseA | sigma-E factor, negative regulatory protein |
| sbp | periplasmic sulfate-binding protein |
| speA | biosynthetic arginine decarboxylase |
| sucB | 2-oxoglutarate dehydrogenase |
| sugE | suppresses groEL, may be chaperone |
| tdcE | probable formate acetyltransferase 3 |
| tdcG | |
| tolC | outer membrane channel |
| trxA | thioredoxin 1 |
| ubiE | 2-octaprenyl-6-methoxy-1,4-benzoquinone --> 2-octaprenyl- |
| | 3-methyl-6-methoxy-1,4-benzoquinone |
| ubiH | 2-octaprenyl-6-methoxyphenol-->2-octaprenyl-6-methoxy-1,4 |
| | benzoquinone |
| ubiX | 3-octaprenyl-4-hydroxybenzoate carboxy-lyase |
| xni | |
| ybbY | putative transport |
| ycfM | orf, hypothetical protein |
| ydeJ | orf, hypothetical protein |
| yeeY | putative transcriptional regulator LYSR-type |
| yfeT | orf, hypothetical protein |
| ygaA | putative 2-component transcriptional regulator |
| ygfZ | orf, hypothetical protein |
| yhdX | putative transport system permease protein |
| yheL | orf, hypothetical protein |
| yheM | orf, hypothetical protein |
| yiaY | putative oxidoreductase |
| yidK | putative cotransporter |
| yihV | putative kinase |
| yjbN | orf, hypothetical protein |
| yjcR | putative membrane protein |
| yjcZ | orf, hypothetical protein |
| ynjD | |
| yqeC | |
| yqiH | putative membrane protein |
| yrfA | orf, hypothetical protein |

The genes identified in bold are the qualified mutants after a secondary screen in a 96 well plate format.

The cells were grown with or without colistin, aliquots were taken at defined time points and serial dilution of the cells plated, colony count allowed to quantify the bactericidal effect of colistin. We noticed that the iscS and ubiH mutants were slow growers and formed small size colonies compared to the parent strain.

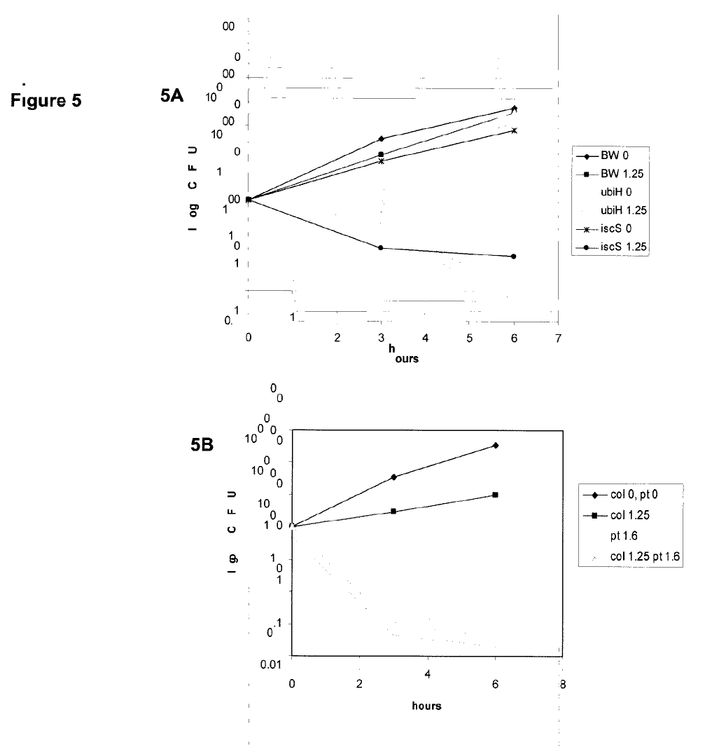

The used amount of colistin was chosen to not affect or little the parent strain, no significant decrease in viability was detected when compared to the non treated cells at the 3 and 5 hour time points. To the contrary, the mutants showed a significant decrease in viability when exposed to colistin, resulting to almost no colony detection at the 5 hour time point (FIG. 5A). The mutants have a slower growth rate and are characterized as forming smaller size colonies (data not shown), the reduced growth rate cannot on its own justify the low colony formation in response to colistin.

There are known inhibitors of the iscS gene product such as iodoacetamine (Urbina et al., 2001) potassium tellurite (Rojas and Vasquez, 2005; Tantalean, 2003). The iscS mutant was shown to be sensitive to potassium tellurite (Rojas and Vasquez, 2005) while ectopic expression of iscS confers resistance to potassium tellurite (Tantalean et al., 2003).

Potassium tellurite is a heavy metal carrier (Te4+) which is known to be toxic for bacteria. It is in fact a known antibacterial agent not use as a therapeutic though and served as a selective agent in microbiological culture medium (e.g. Perez et al., 2007; Zanaroli et al., 2002). Potassium tellurite mediates thiol oxidation in E. coli (Turner et al., 1999). However, the inventors chose to continue experiments with Potassium tellurite based on its ability to inhibit the gene iscS, and not due to any potential anti-bacterial activity it may have. Thus, the inventors demonstrated that inhibition of a gene listed in Table 4, in particular inhibition of iscS by potassium tellurite potentiated colistin effects was a proof of principal that inhibition of the genes listed in table 4 could potentiate antimicrobial activity, such as potentiation of colistin activity.

The inventors used the CFU assay to observe that the amount of colistin used was not affecting the parent strain, no significant decrease in viability was detected when compared to the non treated cells at the 3 hour time point (FIG. 5B). A decrease in viability was noticed at the 5 hour time point. Potassium tellurite at 1.6 ug/ml was not or little affecting the CFU at the 3 hour time point while it shows a stronger growth inhibition at 6 hours. The inventors observed that the combination of colistin and potassium tellurite resulted in a significant decrease in viability closed to a 2 log decrease in average.

The inventors have therefore designed systems biology platform that can be used to discover and identify E. coli mutants which exhibit increased sensitivity to colistin. From the pool of mutant candidates the inventors qualified the strains disrupted on the ubiH and iscS loci. These gene products are incorporated into two major cellular processes. ubiH is involved the process to generate ATP while the iscS gene product is involved the transfer of iron-sulfur clusters.

Iron-Sulfur [Fe—S] clusters are prosthetic groups which are required for crucial biological processes such as electron transfer, iron/sulfur storage, gene regulation, tRNA modification and enzyme activity. The iscS gene product, a cysteine desulfurase, acts mostly as a sulfur donor for other proteins involved in diverse cellular regulatory pathways. iscS obtains sulfur from cysteine which is then converted to alanine and serves as sulfur donor for [Fe—S] cluster assembly. The role of these genes to potentiate colistin is likely related to a more global protein functionality aspect. These Fe—S cluster groups are important for many cellular processes such as tRNA modification, regulation of enzyme activity, substrate binding and activation as well as regulation of gene expression. The Fe—S clusters main biochemical roles are (1) as acceptor and donor of electrons and (2) as binder of the oxygen nitrogen groups of diverse substrates (review in Inlay, 2006). Consequently their roles are linked into the process to synthesize ATP.

Such an approach can lead to a combination therapy involving colistin and selected compounds which can ultimately reduce the toxic adverse effect of colistin either by reducing the colistin dosage and/or by reducing the treatment length.

### REFERENCES

The references cited herein and throughout the application are incorporated herein by reference.

Baba T, Ara T, Hasegawa M, Takai Y, Okumura Y, Baba M, Datsenko K A, Tomita M, Wanner B L, Mori H. Mol Syst Biol. 2:2006.0008 (2006).

Falagas M E and Kasakiou S K Clin Infect Dis 40: 1333-1341 (2005).

Imlay J A. Mol Microbiol. 59:1073-82 (2006).

Lauhon J Bacteriol. 184:6820-6829 (2002).

Li J, Nation R L, Turnidge J D, Milne R W, Coulthard K, Rayner C R, Paterson D L. Lancet Infect Dis. 6:589-601 (2006).

Perez J M, Calderon L, Arenas F A, Fuentes D E, Pradenas G A, Fuentes E L, Sandoval J M, Castro M E, Elias A O, Vasquez C C. PLoS ONE. 14;2:e211 (2007).

Rojas D M, Vasquez C C. Res Microbiol. 156:465-71 (2005).

Song J Y, Kee S Y, Hwang I S, Seo Y B, Jeong H W, Kim W J, Cheong H J. J Antimicrob Chemother. 60:317-322 (2007).

Tan T Y, Ng L S, Tan E, Huang G. J Antimicrob Chemother. 60:421-423 (2007).

Tantalean J C, Araya M A, Saavedra C P, Fuentes D E, Perez J M, Calderon I L, Youderian P, Vasquez C C. J Bacteriol. 185:5831-7 (2003).

Turner R J, Weiner J H, Taylor D E. Microbiology. 145:2549-2557 (1999).

Urbina H D, Silberg J J, Hoff K G, Vickery L E. J. Biol. Chem., 276:44521-44526 (2001).

Young I G, Stroobant P, Macdonald C G, Gibson F. J Bacteriol. 114:42-52 (1973).

Zanaroli G, Fedi S, Carnevali M, Fava F, Zannoni D. Res Microbiol. 153:353-360 (2002).

1. A composition comprising an antimicrobial agent and an enhancer to the antimicrobial agent, wherein the enhancer to the antimicrobial agent is an inhibitor of a gene product that by inactivating the gene product potentiates the effectiveness of the antimicrobial agent.

2. The composition of claim 1, wherein the antimicrobial agent is an antimicrobial peptide.

3. The composition of claim 2, wherein the antimicrobial peptide is a lipopeptide.

4. The composition of claim 3, wherein the lipopeptide is a cyclic lipopeptide.

5. The composition of claim 4, wherein the cyclic lipopeptide is a polymyxin class of antibiotic or derivative thereof.

6. The composition of claim 5, wherein the polymyxin is selected from the group of polymyxin A, B1, B2, D1, D2, E1 and/or E2, F, G, M, P, S and/or T

7. The composition of claim 5, wherein the polymyxin is selected from polymyxin B1, polymyxin B2, and a mixture of polymyxin B1 and polymyxin B2.

8. The composition of claim 5, wherein the polymyxin is selected from colistin A, colistin B, and a mixture of colistin A and colistin B.

9. The composition of claim 5, wherein the polymyxin is in the form of a colistin salt.

10. The composition of claim 9, wherein the colistin salt is a methane sulphonate and/or sulfate salt.

11. The composition of claim 1, wherein the gene product is selected from a group consisting of agaA, atpA, atpC, atpB, atpD, atpE, atpG, atpH, betB, csdA, csdB, fepC, guaA, guaB, iscS, kdgK, lipA, lysA, mnmA, nuvC, papa, pdxH, phnL, potE, rpiA, sucB, trxA, tusB (YheL), tusE, ubiE, ubiH, uncA, visB, yeeY, yiaY, yidK, yihV, yfhO, yjbN and/or ynjD or homologues, variants or fragments thereof.

12. The composition of claim 1, wherein the inhibitor is selected from a group consisting of mefloquine, venturicidin A, diaryquinoline, betaine aldehyde chloride, acivein, psicofuraine, buthionine sulfoximine, diaminopemelic acid, 4-phospho-D-erythronhydroxamic acid, motexafin gadolinium and/or xycitrin or modified versions or analogues thereof.

13. The composition of claim 1, wherein the gene product is atpA, atpF or atpH or homologues, variants or fragments thereof, and the inhibitor is mefloquine and/or venturicidin A and/or diaryquinoline or modified versions or analogues thereof.

14. The composition of claim 1, wherein the gene product is betB or homologues or variants thereof, and the inhibitor is betaine aldehyde chloride or modified versions or analogues thereof.

15. The composition of claim 1, wherein the gene product is guaA or guaB or homologues or variants thereof, and the inhibitor is acivin and/or psicofluranine or modified versions or analogues thereof.

16. The composition of claim 1, wherein the gene product is LipA or homologues or variants thereof, and the inhibitor is buthionine sulfoximine or modified versions or analogues thereof.

17. The composition of claim 1, wherein the gene product is LysA or homologues or variants thereof, and the inhibitor is diaminopimelic acid or modified versions or analogues thereof.

18. The composition of claim 1, wherein the gene product is rpiA or homologues or variants thereof, and the inhibitor is 4-phospho-D-erythronhydrixamic acid or modified versions or analogues thereof.

19. The composition of claim 1, wherein the gene product is trxA or homologues or variants thereof, and the inhibitor is motexafin gadolinium and/or xycitrin acid or modified versions or analogues thereof.

20. The composition of claim 1, wherein the inhibitor comprises a small molecule, nucleic acid, nucleic acid analogue, peptide, ribosome, antibody, and variants and fragments thereof.

21. (canceled)

22. (canceled)

23. The composition of claim 1, wherein the microorganism is selected from the group consisting of; a bacterium, a gram-positive bacterium, a gram-negative bacterium, a multi-drug resistant microorganism.

24. (canceled)

25. (canceled)

26. (canceled)

27. The composition of claim 23, wherein the multi-drug resistant microorganism is resistant to at least one member of the polymyxin class of antibiotics or derivatives or analogues thereof.

28. (canceled)

29. (canceled)

30. (canceled)

31. The composition of claim 1 further comprising a pharmaceutically acceptable carrier.

32. (canceled)

33. The composition of claim 1, where the amount of the antimicrobial agents is at least 25% less than the same antimicrobial agent in an isogenic cell except for the addition of the enhancer of antimicrobial agent without reduction of antimicrobial effect.

34. (canceled)

35. A method of treatment and/or prophylaxis of an infection caused by an microorganism comprising steps of administering to a subject in need thereof an effective amount of the composition according to claim 1.

36. The method of claim 35, wherein the subject is mammalian, avian, amphibian or a plant.

37. The method of claim 36, wherein the mammalian is human.

38.-41. (canceled)

42. The method of claim 35, wherein the infection is selected from the group consisting of bacterial wound infections, mucosal infections, enteric infections, septic conditions, infectious in airways, cerebrospinal fluid, blood, eyes and skin.

43.-51. (canceled)

52. A method for identifying gene products, wherein inactivation of the gene products potentates antimicrobial agent activity, the method comprising the steps of;
(a) mutating one or more genes in a cell,
(b) contacting the cell with the antimicrobial agent,
(c) incubating the cell for a sufficient amount of time to allow for growth, and;
(d) assessing the number of cells, wherein the number of cells is compared to steps (a)-(c) performed on a non-mutated cell, wherein the decrease in numbers of cells identifies a gene product that when inactivated potentiates antimicrobial peptide activity.

53. The method of claim 52, wherein the cell is bacterium.

54.-58. (canceled)


SEQUENCE LISTING (42 sequences)

SEQ ID NO: 1 - protein, 167 aa, Escherichia coli
MHCYNGMTGLHHREPGMVGAGLTDKRAWLELIADGHHVHPAAMSLCCCCAKERIVLITDAMQAAGMPDGRYTLCGEEVQMHGGVVRTASGGLAGSTLSVDAAVRNMVELTGVTPAEAIHMASLHPARMLGVDGVLGSLKPGKRARVVALDSGLHVQQIWIQGQLASF

SEQ ID NO: 2 - protein, 513 aa, Escherichia coli
MQLNSTEISELIKQRIAQFNVVSEAHNEGTIVSVSDGVIRIHGLADCMQGEMISLPGNRYAIALNLERDSVGAVVMGPYADLAEGMKVKCTGRILEVPVGRGLLGRVVNTLGAPIDGKGPLDHDGFSAVEAIAPGVIERQSVDQPVQTGYKAVDSMIPIGRGQRELIIGDRQTGKTALAIDAIINQRDSGIKCIYVAIGQKASTISNVVRKLEEHGALANTIVVVATASESAALQYLAPYAGCAMGEYFRDRGEDALIIYDDLSKQAVAYRQISLLLRRPPGREAFPGDVFYLHSRLLERAARVNAEYVEAFTKGEVKGKTGSLTALPIIETQAGDVSAFVPTNVISITDGQIFLETNLFNAGIRPAVNPGISVSRVGGAAQTKIMKKLSGGIRTALAQYRELAAFSQFASDLDDATRKQLDHGQKVTELLKQKQYAPMSVAQQSLVLFAAERGYLADVELSKIGSFEAALLAYVDRDHAPLMQEINQTGGYNDEIEGKLKGILDSFKATQSW

SEQ ID NO: 3 - protein, 271 aa, Escherichia coli
MASENMTPQDYIGHHLNNLQLDLRTFSLVDPQNPPATFWTINIDSMFFSVVLGLLFLVLFRSVAKKATSGVPGKFQTAIELVIGFVNGSVKDMYHGKSKLIAPLALTIFVWVFLMNLMDLLPIDLLPYIAEHVLGLPALRVVPSADVNVTLSMALGVFILILFYSIKMKGIGGFTKELTLQPFNHWAFIPVNLILEGVSLLSKPVSLGLRLFGNMYAGELIFILIAGLLPWWSQWILNVPWAIFHILIITLQAFIFMVLTIVYLSMASEEH

SEQ ID NO: 4 - protein, 139 aa, Escherichia coli
MAMTYHLDVVSAEQQMFSGLVEKIQVTGSEGELGIYPGHAPLLTAIKPGMIRIVKQHGHEEFIYLSGGILEVQPGNVTVLADTAIRGQDLDEARAMEAKRKAEEHISSSHGDVDYAQASAELAKAIAQLRVIELTKKAM

SEQ ID NO: 5 - protein, 460 aa, Escherichia coli
MATGKIVQVIGAVVDVEFPQDAVPRVYDALEVQNGNERLVLEVQQQLGGGIVRTIAMGSSDGLRRGLDVKDLEHPIEVPVGKATLGRIMNVLGEPVDMKGEIGEEERWAIHRAAPSYEELSNSQELLETGIKVIDLMCPFAKGGKVGLFGGAGVGKTVNMMELIRNIAIEHSGYSVFAGVGERTREGNDFYHEMTDSNVIDKVSLVYGQMNEPPGNRLRVALTGLTMAEKFRDEGRDVLLFVDNIYRYTLAGTEVSALLGRMPSAVGYQPTLAEEMGVLQERITSTKTGSITSVQAVYVPADDLTDPSPATTFAHLDATVVLSRQIASLGIYPAVDPLDSTSRQLDPLVVGQEHYDTARGVQSILQRYQELKDIIAILGMDELSEEDKLVVARARKIQRFLSQPFFVAEVFTGSPGKYVSLKDTIRGFKGIMEGEYDHLPEQAFYMVGSIEEAVEKAKKL

SEQ ID NO: 6 - protein, 79 aa, Escherichia coli
MENLNMDLLYMAAAVMMGLAAIGAAIGIGILGGKFLEGAARQPDLIPLLRTQFFIVMGLVDAIPMIAVGLGLYVMFAVA

SEQ ID NO: 7 - protein, 287 aa, Escherichia coli
MAGAKEIRSKIASVQNTQKITKAMEMVAASKMRKSQDRMAASRPYAETMRKVIGHLAHGNLEYKHPYLEDRDVKRVGYLVVSTDRGLCGGLNINLFKKLLAEMKTWTDKGVQCDLAMIGSKGVSFFNSVGGNVVAQVTGMGDNPSLSELIGPVKVMLQAYDEGRLDKLYIVSNKFINTMSQVPTISQLLPLPASDDDDLKHKSWDYLYEPDPKALLDTLLRRYVESQVYQGVVENLASEQAARMVAMKAATDNGGSLIKELQLVYNKARQASITQELTEIVSGAAAV

SEQ ID NO: 8 - protein, 177 aa, Escherichia coli
MSEFITVARPYAKAAFDFAVEHQSVERWQDMLAFAAEVTKNEQMAELLSGALAPETLAESFIAVCGEQLDENGQNLIRVMAENGRLNALPDVLEQFIHLRAVSEATAEVDVISAAALSEQQLAKISAAMEKRLSRKVKLNCKIDKSVMAGVIIRAGDMVIDGSVRGRLERLADVLQS

SEQ ID NO: 9 - protein, 490 aa, Escherichia coli
MSRMAEQQLYIHGGYTSATSGRTFETINPANGNVLATVQAAGREDVDRAVKSAQQGQKIWASMTAMERSRILRRAVDILRERNDELAKLETLDTGKAYSETSTVDIVTGADVLEYYAGLIPALEGSQIPLRETSFVYTRREPLGVVAGIGAWNYPIQIALWKSAPALAAGNAMIFKPSEVTPLTALKLAEIYSEAGLPDGVFNVLPGVGAETGQYLTEHPGIAKVSFTGGVASGKKVMANSAASSLKEVTMELGGKSPLIVFDDADLDLAADIAMMANFFSSGQVCTNGTRVFVPAKCKAAFEQKILARVERIRAGDVFDPQTNFGPLVSFPHRDNVLRYIAKGKEEGARVLCGGDVLKGDGFDNGAWVAPTVFTDCSDDMTIVREEIFGPVMSILTYESEDEVIRRANDTDYGLAAGIVTADLNRAHRVIHQLEAGICWINTWGESPAEMPVGGYKHSGIGRENGVMTLQSYTQVKSIQVEMAKFQSIF

SEQ ID NO: 10 - protein, 401 aa, Escherichia coli
MNVFNPAQFRAQFPALQDAGVYLDSAATALKPEAVVEATQQFYSLSAGNVHRSQFAEAQRLTARYEAAREKVAQLLNAPDDKTIVWTRGTTESINMVAQCYARPRLQPGDEIIVSVAEHHANLVPWLMVAQQTGAKVVKLPLNAQRLPDVDLLPELITPRSRILALGQMSNVTGGCPDLARAITFAHSAGMVVMVDGAQGAVHFPADVQQLDIDFYAFSGHKLYGPTGIGVLYGKSELLEAMSPWLGGGKMVHEVSFDGFTTQSAPWKLEAGTPNVAGVIGLSAALEWLADYDINQAESWSRSLATLAEDALAKRPGFRSFRCQDSSLLAFDFAGVHHSDMVTLLAEYGIALRAGQHCAQPLLAELGVTGTLRASFAPYNTKSDVDALVNAVDRALELLVD

SEQ ID NO: 11 - protein, 646 aa, Escherichia coli
MMSYVDWPPLILRHTYYMAEFETTFADLGLKAPILEALNDLGYEKPSPIQAECIPHLLNGRDVLGMAQTGSGKTAAFSLPLLQNLDPELKAPQILVLAPTRELAVQVAEAMTDFSKHMRGVNVVALYGGQRYDVQLRALRQGPQIVVGTPGRLLDHLKRGTLDLSKLSGLVLDEADEMLRMGFIEDVETIMAQIPEGHQTALFSATMPEAIRRITRRFMKEPQEVRIQSSVTTRPDISQSYWTVWGMRKNEALVRFLEAEDFDAAIIFVRTKNATLEVAEALERNGYNSAALNGDMNQALREQTLERLKDGRLDILIATDVAARGLDVERISLVVNYDIPMDSESYVHRIGRTGRAGRAGRALLFVENRERRLLRNIERTMKLTIPEVELPNAELLGKRRLEKFAAKVQQQLESSDLDQYRALLSKIQPTAEGEELDLETLAAALLKMAQGERTLIVPPDAPMRPKREFRDRDDRGPRDRNDRGPRGDREDRPRRERRDVGDMQLYRIEVGRDDGVEVRHIVGAIANEGDISSRYIGNIKLFASHSTIELPKGMPGEVLQHFTRTRILNKPMNMQLLGDAQPHTGGERRGGGRGFGGERREGGRNFSGERREGGRGDGRRFSGERREGRAPRRDDSTGRRRFGGDA

SEQ ID NO: 12 - protein, 406 aa, Escherichia coli
MIFSVDKVRADFPVLSREVNGLPLAYLDSAASAQKPSQVIDAEAEFYRHGYAAVHRGIHTLSAQATEKMENVRKRASLFINARSAEELVFVRGTTEGINLVANSWGNSNVRAGDNIIISQMEHHANIVPWQMLCARVGAELRVIPLNPDGTLQLETLPTLFDEKTRLLAITHVSNVLGTENPLAEMITLAHQHGAKVLVDGAQAVMHHPVDVQALDCDFYVFSGHKLYGPTGIGILYVKEALLQEMPPWEGGGSMIATVSLSEGTTWTKAPWRFEAGTPNTGGIIGLGAALEYVSALGLNNIAEYEQNLMHYALSQLESVPDLTLYGPQNRLGVIAFNLGKHHAYDVGSFLDNYGIAVRTGHHCAMPLMAYYNVPAMCRASLAMYNTHEEVDRLVTGLQRIHRLLG

SEQ ID NO: 13 - protein, 271 aa, Escherichia coli
MTESVARLRGEQLTLGYGKYTVAENLTVEIPDGHFTAIIGPNGCGKSTLLRTLSRLMTPAHGHVWLDGEHIQHYASKEVARRIGLLAQNATTPGDITVQELVARGRYPHQPLFTRWRKEDEEAVTKAMQATGITHLADQSVDTLSGGQRQRAWIAMVLAQETAIMLLDEPTTWLDISHQIDLLELLSELNREKGYTLAAVLHDLNQACRYASHLIALREGKIVAQGAPKEIVTAELIERIYGLRCMIIDDPVAGTPLVVPLGRTAPSTANS

SEQ ID NO: 14 - protein, 525 aa, Escherichia coli
MTENIHKHRILILDFGSQYTQLVARRVRELGVYCELWAWDVTEAQIRDFNPSGIILSGGPESTTEENSPRAPQYVFEAGVPVFGVCYGMQTMAMQLGGHVEASNEREFGYAQVEVVNDSALVRGIEDALTADGKPLLDVWMSHGDKVTAIPSDFITVASTESCPFAIMANEEKRFYGVQFHPEVTHTRQGMRMLERFVRDICQCEALWTPAKIIDDAVARIREQVGDDKVILGLSGGVDSSVTAMLLHRAIGKNLTCVFVDNGLLRLNEAEQVLDMFGDHFGLNIVHVPAEDRFLSALAGENDPEAKRKIIGRVFVEVFDEEALKLEDVKWLAQGTIYPDVIESAASATGKAHVIKSHHNVGGLPKEMKMGLVEPLKELFKDEVRKIGLELGLPYDMLYRHPFPGPGLGVRVLGEVKKEYCDLLRRADAIFIEELRKADLYDKVSQAFTVFLPVRSVGVMGDGRKYDWVVSLRAVETIDFMTAHWAHLPYDFLGRVSNRIINEVNGISRVVYDISGKPPATIEWE

SEQ ID NO: 15 - protein, 488 aa, Escherichia coli
MLRIAKEALTFDDVLLVPAHSTVLPNTADLSTQLTKTIRLNIPMLSAAMDTVTEARLAIALAQEGGIGFIHKNMSIERQAEEVRRVKKHESGVVTDPQTVLPTTTLREVKELTERNGFAGYPVVTEENELVGIITGRDVRFVTDLNQPVSVYMTPKERLVTVREGEAREVVLAKMHEKRVEKALVVDDEFHLIGMITVKDFQKAERKPNACKDEQGRLRVGAAVGAGAGNEERVDALVAAGVDVLLIDSSHGHSEGVLQRIRETRAKYPDLQIIGGNVATAAGARALAEAGCSAVKVGIGPGSICTTRIVTGVGVPQITAVADAVEALEGTGIPVIADGGIRFSGDIAKAIAAGASAVMVGSMLAGTEESPGEIELYQGRSYKSYRGMGSLGAMSKGSSDRYFQSDNAADKLVPEGIEGRVAYKGRLKEIIHQQMGGLRSCMGLTGCGTIDELRTKAEFVRISGAGIQESHVHDVTITKESPNYRLGS

SEQ ID NO: 16 - protein, 412 aa, Escherichia coli
MMYGVYRAMKLPIYLDYSATTPVDPRVAEKMMQFMTMDGTFGNPASRSHRFGWQAEEAVDIARNQIADLVGADPREIVFTSGATESDNLAIKGAANFYQKKGKHIITSKTEHKAVLDTCRQLEREGFEVTYLAPQRNGIIDLKELEAAMRDDTILVSIMHVNNEIGVVQDIAAIGEMCRARGIIYHVDATQSVGKLPIDLSQLKVDLMSFSGHKIYGPKGIGALYVRRKPRVRIEAQMHGGGHERGMRSGTLPVHQIVGMGEAYRIAKEEMATEMERLRGLRNRLWNGIKDIEEVYLNGDLEHGAPNILNVSFNYVEGESLIMALKDLAVSSGSACTSASLEPSYVLRALGLNDELAHSSIRFSLGRFTTEEEIDYTIELVRKSIGRLRDLSPLWEMYKQGVDLNSIEWAHH

SEQ ID NO: 17 - protein, 382 aa, Escherichia coli
MFKSDAARTRQRLSARKGRTLPPGRGKFPHSTTESFRNTFWLKKIMEHCFNMVDQQTTTAQTNANFLQIRFTTMSKKIAVIGECMIELSEKGADVKRGFGGDTLNTSVYIARQVDPAALTVHYVTALGTDSFSQQMLDAWHGENVDTSLTQRMENRLPGLYYIETDSTGERTFYYWRNEAAAKFWLESEQSAAICEELANFDYLYLSGISLAILSPTSREKLLSLLRECRANGGKVIFDNNYRPRLWASKEETQQVYQQMLECTDIAFLTLDDEDALWGQQPVEDVIARTHNAGVKEVVVKRGADSCLVSIAGEGLVDVPAVKLPKEKVIDTTAAGDSFSAGYLAVRLTGGSAEDAAKRGHLTASTVIQYRGAIIPREAMPA

SEQ ID NO: 18 - protein, 321 aa, Escherichia coli
MSKPIVMERGVKYRDADKMALIPVKNVATEREALLRKPEWMKIKLPADSTRIQGIKAAMRKNGLHSVCEEASCPNLAECFNHGTATFMILGAICTRRCPFCDVAHGRPVAPDANEPVKLAQTIADMALRYVVITSVDRDDLRDGGAQHFADCITAIREKSPQIKIETLVPDFRGRMDRALDILTATPPDVFNHNLENVPRIYRQVRPGADYNWSLKLLERFKEAHPEIPTKSGLMVGLGETNEEIIEVMRDLRRHGVTMLTLGQYLQPSRHHLPVQRYVSPDEFDEMKAEALAMGFTHAACGPFVRSSYHADLQAKGMEVK

SEQ ID NO: 19 - protein, 420 aa, Escherichia coli
MPHSLFSTDTDLTAENLLRLPAEFGCPVWVYDAQIIRRQIAALKQFDVVRFAQKACSNIHILRLMREQGVKVDSVSLGEIERALAAGYNPQTHPDDIVFTADVIDQATLERVSELQIPVNAGSVDMLDQLGQVSPGHRVWLRVNPGFGHGHSQKTNTGGENSKHGIWYTDLPAALDVIQRHHLQLVGIHMHIGSGVDYAHLEQVCGAMVRQVIEFGQDLQAISAGGGLSVPYQQGEEAVDTEHYYGLWNAAREQIARHLGHPVKLEIEPGRFLVAQSGVLITQVRSVKQMGSRHFVLVDAGFNDLMRPAMYGSYHHISALAADGRSLEHAPTVETVVAGPLCESGDVFTQQEGGNVETRALPEVKAGDYLVLHDTGAYGASMSSNYNSRPLLPEVLFDNGQARLIRRRQTIEELLALELL

SEQ ID NO: 20 - protein, 368 aa, Escherichia coli
MSETAKKVIVGMSGGVDSSVSAWLLQQQGYQVEGLFMKNWEEDDGEEYCTAAADLADAQAVCDKLGIELHTVNFAAEYWDNVFELFLAEYKAGRTPNPDILCNKEIKFKAFLEFAAEDLGADYIATGHYVRRADVDGKSRLLRGLDSNKDQSYFLYTLSHEQIAQSLFPVGELEKPQVRKIAEDLGLVTAKKKDSTGICFIGERKFREFLGRYLPAQPGKIITVDGDEIGEHQGLMYHTLGQRKGLGIGGTKEGTEEPWYVVDKDVENNILVVAQGHEHPRLMSVGLIAQQLHWVDREPFTGTMRCTVKTRYRQTDIPCTVKALDDDRIEVIFDEPVAAVTPGQSAVFYNGEVCLGGGIIEQRLPLPV

SEQ ID NO: 21 - protein, 404 aa, Escherichia coli
MKLPIYLDYSATTPVDPRVAEKMMQFMTMDGTFGNPASRSHRFGWQAEEAVDIARNQIADLVGADPREIVFTSGATESDNLAIKGAANFYQKKGKHIITSKTEHKAVLDTCRQLEREGFEVTYLAPQRNGIIDLKELEAAMRDDTILVSIMHVNNEIGVVQDIAAIGEMCRARGIIYHVDATQSVGKLPIDLSQLKVDLMSFSGHKIYGPKGIGALYVRRKPRVRIEAQMHGGGHERGMRSGTLPVHQIVGMGEAYRIAKEEMATEMERLRGLRNRLWNGIKDIEEVYLNGDLEHGAPNILNVSFNYVEGESLIMALKDLAVSSGSACTSASLEPSYVLRALGLNDELAHSSIRFSLGRFTTEEEIDYTIELVRKSIGRLRDLSPLWEMYKQGVDLNSIEWAHH

SEQ ID NO: 22 - protein, 513 aa, Escherichia coli; identical to SEQ ID NO: 2

SEQ ID NO: 23 - protein, 218 aa, Escherichia coli
MSDNDELQQIAHLRREYTKGGLRRRDLPADPLTLFERWLSQACEAKLADPTAMVVATVDEHGQPYQRIVLLKHYDEKGMVFYTNLGSRKAHQIENNPRVSLLFPWHTLERQVMVIGKAERLSTLEVMKYFHSRPRDSQIGAWVSKQSSRISARGILESKFLELKQKFQQGEVPLPSFWGGFRVSLEQIEFWQGGEHRLHDRFLYQRENDAWKIDRLAP

SEQ ID NO: 24 - protein, 226 aa, Escherichia coli
MINVQNVSKTFILHQQNGVRLPVLNRASLTVNAGECVVLHGHSGSGKSTLLRSLYANYLPDEGQIQIKHGDEWVDLVTAPARKVVEIRKTTVGWVSQFLRVIPRISALEVVMQPLLDTGVPREACAAKAARLLTRLNVPERLWHLAPSTFSGGEQQRVNIARGFIVDYPILLLDEPTASLDAKNSAAVVELIREAKTRGAAIVGIFHDEAVRNDVADRLHPMGASS

SEQ ID NO: 25 - protein, 439 aa, Escherichia coli
MSQAKSNKMGVVQLTILTMVNMMGSGIIMLPTKLAEVGTISIISWLVTAVGSMALAWAFAKCGMFSRKSGGMGGYAEYAFGKSGNFMANYTYGVSLLIANVAIAISAVGYGTELLGASLSPVQIGLATIGVLWICTVANFGGARITGQISSITVWGVIIPVVGLCIIGWFWFSPTLYVDSWNPHHAPFFSAVGSSIAMTLWAFLGLESACANTDVVENPERNVPIAVLGGTLGAAVIYIVSTNVIAGIVPNMELANSTAPFGLAFAQMFTPEVGKVIMALMVMSCCGSLLGWQFTIAQVFKSSSDEGYFPKIFSRVTKVDAPVQGMLTIVIIQSGLALMTISPSLNSQFNVLVNLAVVTNIIPYILSMAALVIIQKVANVPPSKAKVANFVAFVGAMYSFYALYSSGEEAMLYGSIVTFLGWTLYGLVSPRFELKNKHG

SEQ ID NO: 26 - protein, 219 aa, Escherichia coli
MTQDELKKAVGWAALQYVQPGTIVGVGTGSTAAHFIDALGTMKGQIEGAVSSSDASTEKLKSLGIHVFDLNEVDSLGIYVDGADEINGHMQMIKGGGAALTREKIIASVAEKFICIADASKQVDILGKFPLPVEVIPMARSAVARQLVKLGGRPEYRQGVVTDNGNVILDVHGMEILDPIAMENAINAIPGVVTVGLFANRGADVALIGTPDGVKTIVK

SEQ ID NO: 27 - protein, 405 aa, Escherichia coli
MSSVDILVPDLPESVADATVATWHKKPGDAVVRDEVLVEIETDKVVLEVPASADGILDAVLEDEGTTVTSRQILGRLREGNSAGKETSAKSEEKASTPAQRQQASLEEQNNDALSPAIRRLLAEHNLDASAIKGTGVGGRLTREDVEKHLAKAPAKESAPAAAAPAAQPALAARSEKRVPMTRLRKRVAERLLEAKNSTAMLTTFNEVNMKPIMDLRKQYGEAFEKRHGIRLGFMSFYVKAVVEALKRYPEVNASIDGDDVVYHNYFDVSMAVSTPRGLVTPVLRDVDTLGMADIEKKIKELAVKGRDGKLTVEDLTGGNFTITNGGVFGSLMSTPIINPPQSAILGMHAIKDRPMAVNGQVEILPMMYLALSYDHRLIDGRESVGFLVTIKELLEDPTRLLLDV

SEQ ID NO: 28 - protein, 127 aa, Escherichia coli
MLHQQRNQHARLIPVELYMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 29 - protein, 95 aa, Escherichia coli
MLHTLHRSPWLTDFAALLRLLSEGDELLLLQDGVTAAVDGNRYLESLRNAPIKVYALNEDLIARGLTGQISNDIILIDYTDFVRLTVKHPSQMAW

SEQ ID NO: 30 - protein, 109 aa, Escherichia coli
MLIFEGKEIETDTEGYLKESSQWSEPLAVVIAENEGISLSPEHWEVVRFVRDFYLEFNTSPAIRMLVKAMANKFGEEKGNSRYLYRLFPKGPAKQATKIAGLPKPVKCI

SEQ ID NO: 31 - protein, 251 aa, Escherichia coli
MVDKSQETTHFGFQTVAKEQKADMVAHVFHSVASKYDVMNDLMSFGIHRLWKRFTIDCSGVRRGQTVLDLAGGTGDLTAKFSRLVGETGKVVLADINESMPKMGREKLRNIGVIGNVEYVQANAEALPFPDNTFDCITISFGLRNVTDKDKALRSMYRVLKPGGRLLVLEFSKPIIEPLSKAYDAYSFHVLPRIGSLVANDADSYRYLAESIRMHPDQDTLKAMMQDAGFESVDYYNLTAGVVALHRGYKF

SEQ ID NO: 32 - protein, 392 aa, Escherichia coli [sequence omitted]

SEQ ID NO: 33 - protein, 513 aa, Escherichia coli; identical to SEQ ID NO: 2

SEQ ID NO: 34 - protein, 392 aa, Escherichia coli [sequence omitted]

SEQ ID NO: 35 - protein, 316 aa, Escherichia coli
MHCKGACMKPLLDVLMILDALEKEGSFAAASAKLYKTPSALSYTVHKLESDLNIQLLDRSGHRAKFTRTGKMLLEKGREVLHTVRELEKQAIKLHEGWENELVIGVDDTFPFSLLAPLIEAFYQHHSVTRLKFINGVLGGSWDALTQGRADIIVGAMHEPPSSSEFGFSRLGDLEQVFAVAPHHPLALEEEPLNRRIIKRYRAIVVGDTAQAGASTASQLLDEQEAITVFDFKTKLELQISGLGCGYLPRYLAQRFLDSGALIEKKVVAQTLFEPVWIGWNEQTAGLASGWWRDEILANSAIAGVYAKSDDGKSAI

SEQ ID NO: 36 - protein, 382 aa, Escherichia coli
MAASTFFIPSVNVIGADSLTDAMNMMADYGFTRTLIVTDNMLTKLGMAGDVQKALEERNIFSVIYDGTQPNPTTENVAAGLKLLKENNCDSVISLGGGSPHDCAKGIALVAANGGDIRDYEGVDRSAKPQLPMIAINTTAGTASEMTRFCIITDEARHIKMAIVDKHVTPLLSVNDSSLMIGMPKSLTAATGMDALTHAIEAYVSIAATPITDACALKAVTMIAENLPLAVEDGSNAKAREAMAYAQFLAGMAFNNASLGYVHAMAHQLGGFYNLPHGVCNAVLLPHVQVFNSKVAAARLRDCAAAMGVNVTGKNDAEGAEACINAIRELAKKVDIPAGLRDLNVKEEDFAVLATNALKDACGFTNPIQATHEEIVAIYAAR

SEQ ID NO: 37 - protein, 571 aa, Escherichia coli
MNSLQILSFVGFTLLVAVITWWKVRKTDTGSQQGYFLAGRSLKAPVIAASLMLTNLSTEQLVGLSGQAYKSGMSVMGWEVTSAVTLIFLALIFLPRYLKRGIATIPDFLEERYDKTTRIIIDFCFLIATGVCFLPIVLYSGALALNSLFHVGESLQISHGAAIWLLVILLGLAGILYAVIGGLRAMAVADSINGIGLVIGGLMVPVFGLIAMGKGSFMQGIEQLTTVHAEKLNSIGGPTDPLPIGAAFTGLILVNTFYWCTNQGIVQRTLASKSLAEGQKGALLTAVLKMLDPLVLVLPGLIAFHLYQDLPKADMAYPTLVNNVLPVPMVGFFGAVLFGAVISTFNGFLNSASTLFSMGIYRRIINQNAEPQQLVTVGRKFGFFIAIVSVLVAPWIANAPQGLYSWMKQLNGIYNVPLVTIIIMGFFFPRIPALAAKVAMGIGIISYITINYLVKFDFHFLYVLACTFCINVVVMLVIGFIKPRATPFTFKDAFAVDMKPWKNVKIASIGILFAMIGVYAGLAEFGGYGTRWLAMISYFIAAVVIVYLIFDSWRHRHDPAVTFTPDGKDSL

SEQ ID NO: 38 - protein, 300 aa, Escherichia coli
MTMIRVACVGITVMDRIYYVEGLPTESGKYVARNYTEVGGGPAATAAVAAARLGAQVDFIGRVGDDDTGNSLLAELESWGVNTRYTKRYNQAKSSQSAIMVDTKGERIIINYPSPDLLPDAEWLEEIDFSQWDVVLADVRWHDGAKKAFTLARQAGVMTVLDGDITPQDISELVALSDHAAFSEPGLARLTGVKEMASALKQAQTLTNGHVYVTQGSAGCDWLENGGRQHQPAFKVDVVDTTGAGDVFHGALAVALATSGDLAESVRFASGVAALKCTRPGGRAGIPDCDQTRSFLSLFV

SEQ ID NO: 39 - protein, 412 aa, Escherichia coli; identical to SEQ ID NO: 16

SEQ ID NO: 40 - protein, 345 aa, Escherichia coli
MHGNSEMQKINQTSAMPEKTDVHWSGRFSVAPMLDWTDRHCRYFLRLLSRNTLLYTEMVTTGAIIHGKGDYLAYSEEEHPVALQLGGSDPAALAQCAKLAEARGYDEINLNVGCPSDRVQNGMFGACLMGNAQLVADCVKAMRDVVSIPVTVKTRIGIDDQDSYEFLCDFINTVSGKGECEMFIIHARKAWLSGLSPKENREIPPLDYPRVYQLKRDFPHLTMSINGGIKSLEEAKAHLQHMDGVMVGREAYQNPGILAAVDREIFGSSDTDADPVAVVRAMYPYIERELSQGTYLGHITRHMLGLFQGIPGARQWRRYLSENAHKAGADINVLEHALKLVADKR

SEQ ID NO: 41 - protein, 217 aa, Escherichia coli
MLCVKNVSLRLPESRLLTNVNFTVDKGDIVTLMGPSGCGKSTLFSWMIGALAEQFSCTGELWLNEQRIDILPTAQRQIGILFQDALLFDQFSVGQNLLLALPATLKGNARRNAVNDALERSGLEGAFHQDPATLSGGQRARVALLRALLAQPKALLLDEPFSRLDVALRDNFRQWVFSEVRALAIPVVQVTHDLQDVPADSSVLDMAQWSENYNKLR

SEQ ID NO: 42 - protein, 5 aa, Artificial Sequence
  Description of Artificial Sequence Synthetic peptide
GGGGC